(19) United States
(12) Patent Application Publication
(10) Pub. No.: US 2008/0132477 A1
(43) Pub. Date: Jun. 5, 2008

(54) Macrocyclic Compounds Useful as Bace Inhibitors

(76) Inventors: Claudia Betschart, Basel (CH); Andreas Lerchner, Binningen (CH); Rainer Machauer, Freiburg (DE); Heinrich Rueger, Flueh (CH); Marina Tintelnot-Blomley, Maulburg (DE); Siem Jacob Veenstra, Lorrach (DE)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/794,413

(22) PCT Filed: Jan. 13, 2006
(86) PCT No.: PCT/EP06/00280

§ 371 Date: Jun. 26, 2007

(30) Foreign Application Priority Data
Jan. 13, 2005 (GB) 0500682.0
Oct. 4, 2005 (GB) 0520165.2

(51) Int. Cl. A61K 31/395; C07D 487/02

(52) U.S. Cl. 514/183; 540/455; 540/460

(57) ABSTRACT

The invention relates to novel macrocyclic compounds of the formula (I), in which all of the variables are as defined in the specification, the number of ring atoms included in the macrocyclic ring being 14, 15, 16 or 17, in free base form or in acid addition salt form, to their preparation, to their use as medicaments and to medicaments comprising them.

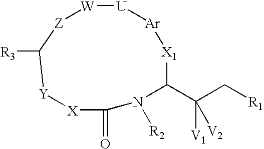

The present invention relates to novel macrocyclic compounds, to their preparation, to their use as medicaments and to medicaments comprising them.

More particularly the invention relates to a compound of the formula

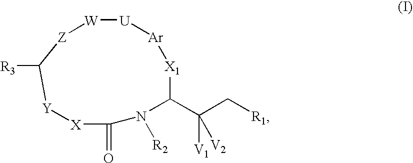

in which
R₁ is CH(Rₑ)C(═O)N(Rₐ)R_{b} or (CH₂)ₖN(R_{c})R_{d}, in which
   k is 0, 1 or 2;
   Rₐ and R_{b}, independently, are hydrogen or an optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl or heteroaryl(C₁₋₄)alkyl group,
   R_{c} and R_{d}, independently, are hydrogen or an optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl, hetero-aryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinolin-4-yl, 1,2,3,4-tetrahydro-isoquinolin-4-yl, 1,2,3,4-tetrahydro-naphthalen-1-yl, 1,1-dioxo-1,2,3,4-tetrahydro-1lambda*6*-ben-zo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1 lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxe-pin-5-yl or 1,3,4,5-tetrahydro-benzo[c]oxepin-5-yl group, or
   Rₐ and R_{b}, or R_{c} and R_{d}, together with the nitrogen to which they are attached, form an optionally substituted pyrrolidinyl, 1-piperidinyl, 4-morpholinyl or piperazinyl group; and
   Rₑ is optionally substituted (C₁₋₈)alkyl, (C₁₋₄)alkoxy(C₁₋₄)alkyl, (C₃₋₇)cycloalkyl or (C₃₋₇)cycloalkyl(C₁₋₄)alkyl;
R₂ is hydrogen or (C₁₋₄)alkyl;
R₃ is hydrogen, (C₁₋₆)alkyl or an optionally substituted (C₁₋₆)alkylOC(═O)NH, (C₃₋₇)cyclo-alkylOC(═O)NH, (C₃₋₇)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₇)cycloalkylC(═O)NH, arylC(═O)NH, aryl(C₁₋₄)alkylC(═O)NH, heteroarylC(═O)NH or heteroaryl(C₁₋₄)al-kylC(═O)NH group;
Ar is an aromatic or heteroaromatic ring, which ring is optionally substituted with halogen, (C₁₋₄)alkoxy, hydroxy or (C₁₋₄)alkyl, whereby U and X₁ are in ortho- or meta-position to each other;
U is a bond, —O—, CF₂, CF₂CF₂, CHF, CHFCHF, cycloprop-1,2-ylene, (C₁₋₃)alkylenoxy, (C₁₋₃)alkylenamino, (C₁₋₈)alkylene or NR_{g}, wherein
   R_{g} is hydrogen, (C₁₋₈)alkyl or (C₃₋₇)cycloalkyl;
   either
V₁ is hydrogen and
V₂ is hydroxy, or
V₁ and V₂ together are oxo;
W is CH═CH, cycloprop-1,2-ylene, CH₂CH(OH), CH(OH)CH₂ or CRₕRₕCRₕRₕ, wherein each Rₕ, independently, is hydrogen, fluorine or (C₁₋₄)alkyl;
X is an optionally substituted (C₁₋₄)alkanylylidene, (C₁₋₄)alkylene, (C₃₋₇)cycloalkylene, piperidin-diyl, pyrrolidin-diyl, benzothiazole-4,6-diyl, benzoxazole-4,6-diyl, 1H-benzotriazole-4,6-diyl, imidazo[1,2-a]pyridine-6,8-diyl, benzo[1,2,5]oxadiazole-4,6-diyl, benzo[1,2,5]thiadiazole-4,6-diyl, 1H-indole-5,7-diyl, 1H-indole-4,6-diyl, 1H-benzimidazole-4,6-diyl or 1H-indazole-1,6-diyl group or an optionally substituted aromatic or heteroaromatic ring, whereby Y and C(═O)NR₂ are in meta-position to each other;
X₁ is CR_{f}R_{f}, wherein
   each R_{f}, independently, is hydrogen, fluorine or an optionally substituted (C₁₋₈)alkyl, (C₁₋₄)alkoxy(C₁₋₄)alkyl, (C₃₋₇)cycloalkyl or (C₃₋₇)cycloalkyl(C₁₋₄)alkyl group;
Y is a bond, O, S(═O)₂, S(═O)₂NR_{g}, N(R_{g})S(═O)₂, NR_{g}, C(R_{g})OH, C(═O)NR_{g}, N(R_{g})C(═O), C(═O)N(R_{g})O or ON(R_{g})C(═O), wherein
   R_{g} is hydrogen, (C₁₋₈)alkyl or (C₃₋₇)cycloalkyl; and
Z is O, CH₂, CF₂, CHF, cycloprop-1,2-ylene or a bond, the number of ring atoms included in the macrocyclic ring being 14, 15, 16 or 17, in free base form or in acid addition salt form.

On account of the asymmetrical carbon atoms present in the compounds of the formula I, the compounds may exist in pure optically active form or in the form of mixtures of optical isomers, e.g. in the form of racemic mixtures. All pure optical isomers and their mixtures, including the racemic mixtures, are part of the present invention.

Halogen denotes fluorine, bromine, chlorine or iodine.

Optional substituents on alkyl, alkoxy or cycloalkyl groups or moieties, or, when Rₐ and R_{b}, or R_{c} and R_{d}, together with the nitrogen, to which they are attached, form a substituted pyrrolidinyl, 1-piperidinyl, 4-morpholinyl or piperazinyl group, on the last mentioned substituted groups, may be one to three groups independently selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxy-carbonyl, (C₁₋₄)alkylcarbonyloxy, (C₁₋₄)alkylcarbonyl, (C₁₋₄)alkylsulfonyl, cyano, oxo and (C₃₋₇)cycloalkyl.

Optional substituents on chroman-4-yl, isochroman-4-yl, thiochroman-4-yl, isothiochroman-4-yl, 1,1-dioxo-1lambda*6*-thiochroman-4-yl, 2,2-dioxo-2lambda*6-isothiochroman-4-yl, 1,2,3,4-tetrahydro-quinolin-4-yl, 1,2,3,4-tetrahydro-isoquinolin-4-yl, 1,2,3,4-tetrahydro-naphthalen-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]-oxathiin-4-yl, 2,3,4,5-tetrahydro-benzo[b]oxepin-5-yl or 1,3,4,5-tetrahydro-benzo[c]oxepin-5-yl, benzothiazole-4,6-diyl, benzoxazole-4,6-diyl, 1H-benzotriazole-4,6-diyl, imidazo-[1,2-a]pyridine-6,8-diyl, benzo[1,2,5]oxadiazole-4,6-diyl, benzo[1,2,5]thiadiazole-4,6-diyl, 1H-indole-5,7-diyl, 1H-indole-4,6-diyl, 1H-benzimidazole-4,6-diyl, 1H-indazole-1,6-diyl, aryl or heteroaryl rings or moieties are one to four, especially one to three, groups independently selected from hydroxy, (C₁₋₈)alkyl, (C₁₋₆)alkoxy, S(═O)₂(C₁₋₄)alkyl, (C₃₋₇)cycloalkyl, (C₃₋₇)cycloalkyl(C₁₋₄)alkyl, cyano, nitro, trifluoromethyl, halogen, (C₁₋₄)alkoxy(C₁₋₄)alkyl optionally substituted by halogen or by (C₁₋₄)alkoxy, (C₁₋₄)alkylcarbonyl(C₁₋₄)alkoxy, (C₁₋₄)alkoxy substituted by optionally substituted carbamoyl, optionally substituted carbamoyloxy, (C₁₋₄)alkylcarbonyloxy, (C₁₋₄)alkoxy(C₁₋₄)alkoxy, (C₁₋₄)alkylcarbonyl(C₁₋₄)alkyl, (C₁₋₄)alkoxycarbonyl, (C₁₋₄)alkylcarbonyl, 2-oxopyrrolidin-1-yl, (C₁₋₄)alkylcarbonylamino optionally substituted at the amino moiety, (C₁₋₄)alkylsulfonylamino optionally substituted at the amino moiety, aryl, heteroaryl and optionally substituted carbamoyl.

When R_{c}, and/or R_{d} is substituted aryl or heteroaryl, optional substituents may further be one to three groups selected from benzyloxy, phenoxy, S(═O)₂NH₂, N(H)S(═O)₂(C₁₋₃)alkyl, carboxy, (C₁₋₄)alkoxycarbonyl, (C₁₋₄)alkylcarbamoyl, (C₁₋₄)alkylsulfonyl, (C₁₋₄)alkylcarbonyloxy, (C₁₋₄)alkylcarbonyl, hydroxy(C₁₋₄)alkyl and optionally substituted amino.

Optional substituents on alkanylylidene, alkylene, alkylenoxy, cycloalkylene, piperidin-diyl or pyrrolidin-diyl groups or moieties may be one to three groups independently selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonyl, (C₁₋₄)alkylsulfonyl, cyano, oxo, carboxy, carbamoyl and (C₃₋₇)cycloalkyl.

Optional substituents on amino groups can be one or two groups independently selected from (C₁₋₄)alkyl, (C₁₋₄)alkoxy(C₁₋₄)alkyl, (C₁₋₄)alkoxycarbonyl, aryl(C₁₋₄)alkoxycarbonyl and heteroaryl(C₁₋₄)alkoxycarbonyl.

Optional substituents on carbamoyl groups or moieties can be one or two groups selected from (C₁₋₄)alkyl and (C₁₋₄)alkoxy(C₁₋₄)alkyl.

Aryl is naphthyl or preferably phenyl. It can also be fused with a cycloalkyl or a heteroaromatic ring (e.g. to form a quinolyl or indolyl group).

Heteroaryl is an aromatic 5- or 6-membered ring, in which 1, 2 or 3 ring atoms are hetero atoms independently selected from O, N and S, such as thiazolyl, oxazolyl, pyrimidinyl or pyridyl, preferably oxazolyl, pyrimidinyl or pyridyl. It can also be fused with a cycloalkyl or an aromatic or heteroaromatic ring (e.g. to form a quinolyl or indolyl group).

Any non-cyclic carbon containing group or moiety with more than 1 carbon atom is straight-chain or branched.

Unless defined otherwise, carbon containing groups, moieties or molecules contain 1 to 8, preferably 1 to 6, more preferably 1 to 4, most preferably 1 or 2, carbon atoms.

In preferred embodiments, the invention relates to a compound of the formula I, in free base form or in acid addition salt form, in which

(1) R₁ is CH(Rₑ)C(═O)N(Rₑ)R_{b} and Rₐ, R_{b} and Rₑ have one of the meanings defined herein-before;
(2) R₁ is CH(Rₑ)C(═O)N(Rₐ)R_{b}, R_{b} and Rₑ have one of the meanings defined herein-before and Rₐ is hydrogen;
(3) R₁ is CH(Rₑ)C(═O)N(Rₐ)R_{b}, Rₐ and Rₑ have one of the meanings defined hereinbefore and R_{b} is (C₁₋₈)alkyl, preferably (C₁₋₅)alkyl, more preferably n-butyl;
(4) R₁ is CH(Rₑ)C(═O)N(Rₐ)R_{b}, Rₐ and R_{b} have one of the meanings defined hereinbefore and Rₑ is (C₁₋₈)alkyl, preferably (C₁₋₄)alkyl, more preferably methyl;
(5) R₁ is (CH₂)ₖN(R_{c})R_{d} and Rₑ, R_{d} and k have one of the meanings defined hereinbefore;
(6) R₁ is (CH₂)ₖN(R_{c})R_{d}, R_{c} and R_{d} have one of the meanings defined hereinbefore and k is 0;
(7) R₁ is (CH₂)ₖN(R_{c})R_{d}, k and R_{d} have one of the meanings defined hereinbefore and R_{c} is hydrogen;
(8) R₁ is (CH₂)ₖN(R_{c})R_{d}, k and R_{d} have one of the meanings defined hereinbefore and R_{d} is hydrogen or an optionally substituted aryl(C₁₋₄)alkyl or heteroaryl(C₁₋₄)alkyl group, preferably an optionally substituted phenyl(C₁₋₄)alkyl, pyridyl(C₁₋₄)alkyl or pyrimidinyl(C₁₋₄)alkyl group,
more preferably an optionally substituted phenyl(C₁₋₁₂)alkyl, pyridyl(C₁₋₂)alkyl group or pyrimidinyl(C₁₋₂)alkyl,
more preferably a phenyl(C₁₋₂)alkyl, pyridyl(C₁₋₂)alkyl or pyrimidinyl(C₁₋₂)alkyl group optionally substituted by 1 to 4 substituents, independently selected from the group, consisting of (C₁₋₈)alkyl and (C₃₋₇)cycloalkyl,
preferably 3-(C₁₋₈)alkylbenzyl, 3-(C₃₋₇)cycloalkylbenzyl, 4-(C₁₋₈)alkylpyrid-2-ylmethyl or 6-(C₁₋₈)alkylpyrimidin-4-ylmethyl,
more preferably 3-isopropylbenzyl, more preferably 3-tert-butylbenzyl, more preferably 3-cyclopropylbenzyl, more preferably 4-isopropylpyrid-2-ylmethyl, more preferably 4-tert-butylpyrid-2-ylmethyl, more preferably 6-ethylpyrimidin-4-ylmethyl;
(9) R₂ is hydrogen;
(10) R₃ is hydrogen;
(11) W is preferably CH═CH, preferably CH₂CH₂;
(12) V₁ is hydrogen;
(13) V₂ is hydroxy;

### (14) X₁ is CH₂;

(15) Ar is preferably unsubstituted 1,3-phenylene, preferably 5-methyl-1,3-phenylene, preferably 4-methoxy-1,3-phenylene, preferably 4-hydroxy-1,3-phenylene, preferably 4-fluoro-1,3-phenylene;
(16) U is preferably O, preferably CH₂, preferably CH₂CH₂, preferably CH₂O, preferably CH₂CH₂O;
(17) Z is a bond;
(18) Y is preferably O, preferably a bond, preferably NH, preferably NCH₃;
(19) X is preferably unsubstituted 1,3-phenylene, preferably substituted 1,3-phenylene, preferably unsubstituted 2,4-pyridylene, preferably unsubstituted 2,4-pyridylene;
(20) X has preferably one of the meanings, which it has in the compounds of the formula I described in the Examples;
(21) the number of ring atoms included in the macrocyclic ring is 14;
(22) the number of ring atoms included in the macrocyclic ring is 15;
(23) the number of ring atoms included in the macrocyclic ring is 16;
(24) the number of ring atoms included in the macrocyclic ring is 17.

The preferred embodiments (1) to (24) are preferred independently, collectively or in any combination or sub-combination.

In especially preferred embodiments, the invention relates to one or more than one of the compounds of the formula I mentioned in the Examples hereinafter, in free base form or in acid addition salt form.

In a further aspect, the invention relates to a process for the preparation of the compounds of the formula I and their salts, comprising the steps of

a) cyclisation by metathesis of a compound of the formula

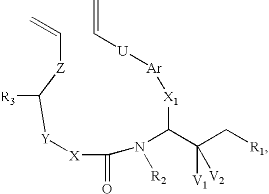

in which all of the variables are as defined for the formula I, in the presence of a catalyst, for instance a ruthenium, tungsten or molybdenum complex, or
b) for the preparation of a compound of the formula I, in which R₁ is CH(Rₑ)C(═O)N(Rₐ)R_{b}, V₁ is hydrogen and V₂ is hydroxy, reaction of a compound of the formula

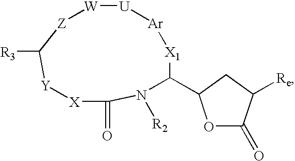

in which all of the variables are as defined for the formula I, with a compound of the formula HN(Rₐ)R_{b} (IV), in which Rₐ and R_{b} are as defined for the formula I, or
c) for the preparation of a compound of the formula I, in which W is CH₂CH₂, hydrogenation of a compound of the formula

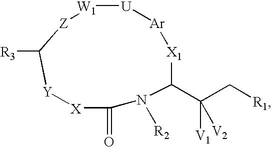

in which W₁ is CH═CH and all of the other variables are as defined for the formula I, or
d) for the preparation of a compound of the formula I, in which R₁ is N(H)R_{d} (in which R_{d}, if it is hydrogen, may be protected by a protecting group to be removed subsequently), V₁ is hydrogen and V₂ is hydroxy, cleavage of the C═O function in the moiety O—C(═O)—NR_{d} in a compound of the formula

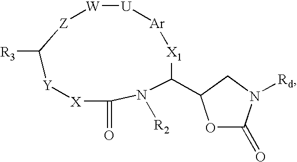

in which all of the variables are as defined for the formula I (and R_{d}, if it is hydrogen, may be protected by a protecting group to be removed subsequently), using, for instance, barium hydroxide or cesium carbonate, or
e) for the preparation of a compound of the formula I, in which R₁ is N(R_{c})R_{d}, V₁ is hydrogen and V₂ is hydroxy, reaction of a compound of the formula

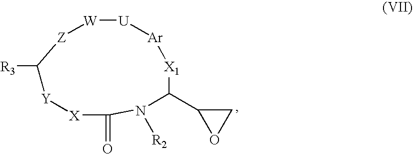

in which all of the variables are as defined for the formula I, with a compound of the formula HN(R_{c})R_{d} (VIII), in which R_{c} and R_{d} are as defined for the formula I, in each case optionally followed by reduction, oxidation or functionalisation of the resulting compound and/or by cleavage of protecting groups optionally present, and of recovering the so obtainable compound of the formula I in free base form or in acid addition salt form.

The reactions can be effected according to conventional methods, for example as described in the Examples.

The working-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, which processes are further aspects of the invention, e.g. as described in the Examples.

The starting materials of the formulae II, III, IV, V, VI, VII and VIII are known or may be prepared according to conventional procedures starting from known compounds, for example as described in the Examples.

Compounds of the formula I and their pharmaceutically acceptable acid addition salts, hereinafter referred to as “agents of the invention”, exhibit valuable pharmacological properties when tested in vitro and in animals, and are therefore useful as medicaments.

The agents of the invention are inhibitors of aspartic proteases and can be used for the treatment of disorders involving processing by such enzymes. Particularly they inhibit beta-secretase and as such inhibit the generation of beta-amyloid and the subsequent aggregation into oligomers and fibrils.

### Test 1: Inhibition of Human BACE

Recombinant BACE (extracellular domain, expressed in baculovirus and purified using standard methods) at 6 nM concentration is incubated with the test compound at various concentrations for 1 hour at room temperature in 100 mM acetate buffer, pH 4.5, containing 0.1% CHAPS. Synthetic peptide substrate Mca-Ser-Glu-Val-Asn-Leu-Asp-Ala-Glu-Phe-Lys(DNP) is added to a final concentration of 3 μM and the increase in fluorescence is recorded at excitation of 325 nm and emission at 400 nm in a microplate spectro-fluorimeter for 20 minutes in 1-minute intervals. IC₅₀ values are calculated from percentage of inhibition of BACE-activity as a function of the test compound concentration.

### Test 2: Inhibition of Human BACE-2

Recombinant BACE-2 (extracellular domain, expressed in baculovirus and purified using standard methods) at 2.5 nM concentrations is incubated with the test compound at various concentrations for 1 hour at room temperature in 100 mM acetate buffer, pH 4.5, containing 0.1% CHAPS. Synthetic peptide substrate Mca-Ser-Glu-Val-Asn-Leu-Asp-Ala-Glu-Phe-Lys(DNP) is added to a final concentration of 3 μM and the increase in fluorescence is recorded at excitation of 325 nm and emission at 400 nm in a microplate spectro-fluorimeter for 20 minutes in 1-minute intervals. IC₅₀ values are calculated from percentage of inhibition of BACE-2-activity as a function of the test compound concentration.

### Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in 100 mM sodium formate buffer, pH 3.1. Synthetic peptide substrate Mca-Gly-Lys-Pro-Ile-Leu-Phe-Phe-Arg-Leu-Lys(DNP)-D-Arg-NH₂ is added to a final concentration of 2 μM and the increase in fluorescence is recorded at excitation of 325 nm and emission at 400 nm in a microplate spectro-fluorimeter for 20 minutes in 1-minute intervals. IC₅₀ values are calculated from percentage of inhibition of cathepsin D-activity as a function of the test compound concentration.

### Test 4: Inhibition of Cellular Release of Amyloid Peptide 1-40

Chinese hamster ovary cells are transfected with the gene for amyloid precursor protein. Cells are plated at a density of 8000 cells/well in a 96-well microtiter plate and cultivated for 24 hours in DMEM cell culture medium containing 10% FCS. The test compound is added to the cells at various concentrations, and cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-indicated tests, the agents of the invention show activity at concentrations below 20 μM.

Specifically, the compound I described in Example 11 shows an IC₅₀ value of 0.04 μM.

The agents of the invention are therefore useful e.g. for the treatment and/or prevention of neurological and vascular disorders related to beta-amyloid generation and/or aggregation, such as neurodegenerative diseases like Alzheimer's disease, Down's Syndrome, memory and cognitive impairment, dementia, amyloid neuropathies, brain inflammation, nerve and brain trauma, vascular amyloidosis, or cerebral haemorrhage with amyloidosis.

Some of the agents of the invention also inhibit BACE2 (beta-site APP-cleaving enzyme 2) or Cathepsin D, close homologues of the pepsin-type aspartyl proteases and of beta-secretase. Due to the correlation of BACE2 and CathD expression with a more tumorigenic and metastatic potential of tumor cells, such inhibitors are useful for the suppression of the metastasis process associated with tumor cells.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range from about 10 to about 2000, preferably from about 10 to about 200, mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a medicament, e.g. for the treatment of neurological or vascular disorders related to beta-amyloid generation and/or aggregation.

The present invention furthermore provides a pharmaceutical composition comprising an agent of the invention in association with at least one pharmaceutical carrier or diluent. Such compositions may be manufactured in conventional manner. Unit dosage forms contain, for example, from about 1 to about 1000, preferably from about 1 to about 500, mg of an agent of the invention.

The agents of the invention can be administered alone or in combination with other pharmaceutical agents effective in the treatment of conditions mentioned above.

The pharmaceutical combination may be in the form of a unit dosage form, whereby each unit dosage will comprise a predetermined amount of the two components, in admixture with suitable pharmaceutical carriers or diluents. Alternatively, the combination may be in form of a package containing the two components separately, e.g. a pack or dispenser-device adapted for the concomitant or separate administration of the two active agents, wherein these agents are separately arranged.

Moreover the present invention provides the use of an agent of the invention, for the manufacture of a medicament for the treatment of any neurological or vascular disorders related to beta-amyloid generation and/or aggregation.

In still a further aspect, the present invention provides a method for the treatment of any neurological or vascular disorders related to beta-amyloid generation and/or aggregation, in a subject in need of such treatment, which comprises administering to such subject a therapeutically effective amount of an agent of the invention.

The following Examples illustrate the invention, but do not limit it.

### EXAMPLES

### Abbreviations

AcCN acetonitrile
AcOH acetic acid
aq. aqueous
BINAP (±)-1,1′-Binaphthaline-2,2′-diyl-bis-(diphenylphosphine)
BOC tert-butoxycarbonyl
conc. concentrated
DBU diazabicycloundecene
DCM dichloromethane
DIPEA diisopropylethylamine
DMAP 4-dimethylaminopyridine
DMF dimethylformamide
DMPU N,N′-dimethylpropylene urea
DMSO dimethylsulfoxide
EDC.HCl 1-ethyl-3-[3-(dimethylamino)propyl]-carbodiimide hydrochloride
ES electron spray
Et₃N triethylamine
Et₂O diethyl ether
EtOAc ethyl acetate
EtOH ethanol
Grubbs II catalyst 1,3-bis-(2,4,6-trimethylphenyl)-2-imidazolidinylidene)dichloro(phenylmethylene)-(tricyclohexylphosphine)ruthenium (CAS 331282-59-8)
h hour(s)
HMDS 1,1,1,3,3,3-hexamethyl-disilazane
¹H-NMR proton nuclear magnetic resonance
HOBt hydroxybenzotriazole
HPLC high pressure liquid chromatography
LAH lithium aluminium hydride
LC liquid chromatography
LHMDS lithium hexamethyldisilazide
MeOH methanol
min minute(s)
MOMCl methoxymethyl chloride
m.p. melting point
MS mass spectroscopy
NH₃ 14 N aqueous ammonia
Pd/C Palladium on charcoal
PL-CHO polymer supported benzaldehyde (3 mmol/g)
PPTS pyridinium-para-toluenesulfonate
Rf retention factor (thin layer chromatography)
rt room temperature
SK-CC02-A 2-(dimethylamino)ferrocen-1-yl-palladium(II)chloride dinorbornylphosphine complex (CAS 614753-51-4)
TBAF tetrabutyl-ammonium floride
TBAI tetrabutyl-ammonium iodide
TBME tert-butyl methyl ether
Tf₂O Trifluoromethanesulfonic anhydride
TFA trifluoroacetic acid
THF tetrahydrofuran

### Example 1

### (2R*,4S*)—N-Butyl-4-hydroxy-4-((S*)-19-methoxy-2-oxo-11-oxa-3,16,18-triaza-tricyclo[15.3.1.1*6,10*]docosa-1(21),6(22),7,9,17,19-hexaen-4-yl)-2-methyl-butyramide

### a) 2-Allylamino-N-[(1S*,2S*,4R*)-1-(3-allyloxy-benzyl)-4-butylcarbamoyl-2-hydroxy-pentyl]-6-methoxy-isonicotinamide

A solution of 300 mg (0.67 mmol) [(1S*,2S*,4R*)-1-(3-allyloxy-benzyl)-4-butylcarbamoyl-2-hydroxy-pentyl]-carbamic acid tert-butyl ester (building block B1) in 4 N HCl in dioxane is stirred at rt for 1 h. The solvent is evaporated and the residue is dried in vacuum. The obtained residue, 167 mg (0.80 mmol, 1.2 eq) 2-allylamino-6-methoxy-isonicotinic acid (building block A4), 0.109 g (0.80 mmol, 1.2 eq) HOBt, 0.192 g (1.0 mmol, 1.5 eq) EDC.HCl and 0.47 ml (3.3 mmol, 5 eq) Et₃N are dissolved in 10 ml DCM and stirred over night at rt. The reaction is diluted with EtOAc and washed with aq. bicarbonate and brine. The organic layer is dried over sodium sulfate and the solvents are evaporated at reduced pressure. The residue is purified by chromatography on silica (flashmaster, DCM to DCM/MeOH 9/1) followed by crystallization from DCM/hexane/ether to give the product.

MS (LC/MS): 561=[M+Na]⁺

¹H-NMR (400 MHz, CDCl₃): 7.22 (t, 1H), 6.88 (d, 1H), 6.85 (s, 1H), 6.80 (d, 1H), 6.54 (d, 1H), 6.23 (d, 2H), 6.11-5.92 (m, 2H), 5.77 (t, 1H), 5.43 (d, 1H), 5.32-5.26 (m, 2H), 5.19 (d, 1H), 4.66 (d, 1H), 4.53 (d, 2H), 4.18 (q, 1H), 4.0-3.95 (m, 2H), 3.88 (s, 3H), 3.88-3.83 (m, 1H), 3.30-3.16 (m, 2H), 3.01 (d, 2H), 2.61-2.53 (m, 1H), 1.75-1.61 (m, 3H), 1.50-1.43 (m, 2H), 1.38-1.29 (m, 2H), 1.16 (d, 3H), 0.93 (t, 3H).

### b) (2R*,4S*)—N-Butyl-4-hydroxy-4-((S*)-19-methoxy-2-oxo-11-oxa-3,16,18-triaza-tricyclo[15.3.1.1*6,10*]docosa-1(21),6(22),7,9,17,19-hexaen-4-yl)-2-methyl-butyramide

To a solution of 183 mg (0.34 mmol) 2-allylamino-N-[(1S*,2S*,4R*)-1-(3-allyloxy-benzyl)-4-butylcarbamoyl-2-hydroxy-pentyl]-6-methoxy-isonicotinamide in 350 ml dry DCM are added under an argon atmosphere 14 mg (0.05 eq) benzylidene(1,3-dimesitylimidazolidin-2-ylidene)(tricyclohexylphosphine)ruthenium dichloride (CAS 246047-72-3) catalyst. The reaction is stirred at reflux temperature (bath temperature 60° C.) for 3 h. After a second addition of catalyst (14 mg) the reaction is heated over night at reflux. The solvent is evaporated and the residue is purified by chromatography on silica (flashmaster, DCM to DCM/MeOH 9/1), followed by crystallization from DCM/MeOH/hexane. The resulting solid is hydrogenated with 46 mg Pd/C (10% Engelhard 4505) in 30 ml methanol/THF (1/1) at rt (1 atm H₂) during 5 h. After filtering through Celite the solvent is evaporated and the crude product is purified by crystallization from DCM/MeOH/hexane to give the product.

MS (LC/MS): 535=[M+Na]⁺

¹H-NMR (400 MHz, d6-DMSO): 8.03 (d, 1H), 7.68 (t, 1H), 7.22 (s, 1H), 7.16 (t, 1H), 6.79 (d, 1H), 6.72 (d, 1H), 6.59 (t, 1H), 5.99 (s, 1H), 5.89 (s, 1H), 3.84 (d, 1H), 4.35-4.3 (m, 1H), 3.9-3.8 (m, 2H), 3.73 (s, 3H), 3.55-3.45 (m, 2H), 3.09-2.97 (m, 3H), 2.82 (dd, 1H), 2.68 (t, 1H), 1.85-1.6 (m, 4H), 1.5-1.18 (m, 7H), 1.04 (d, 3H), 0.88 (t, 3H).

### Example 2

### (2R*,4S*)—N-Butyl-4-hydroxy-4-((S*)-8-methoxy-2-oxo-3,15,17-triaza-tricyclo[14.3.1.1*6,10*]henicosa-1 (20),6(21),7,9,16,18-hexaen-4-yl)-2-methyl-butyramide

### a) 2-Allylamino-N—[(S*)-2-(3-allyl-phenyl)-1-((2S*,4R*)-4-methyl-5-oxo-tetrahydro-furan-2-yl)-ethyl]-6-methoxy-isonicotinamide

A solution of 0.248 g (0.84 mmol) [(S*)-2-(3-ally-phenyl)-1-((2S*,4R*)-4-methyl-5-oxo-tetrahydro-furan-2-yl)-ethyl]-carbamic acid tert-butyl ester (building block B2) in 4 N HCl in dioxane is stirred at rt for 1 h. The solvent is evaporated and the residue is dried in vacuum. The obtained residue, 0.199 g (0.96 mmol, 1.14 eq) 2-allylamino-6-methoxy-isonicotinic acid (building block A4), 0.136 g (1.0 mmol, 1.2 eq) HOBt, 0.241 g (1.26 mmol, 1.5 eq) EDC.HCl, and 0.58 ml (4.2 mmol, 5 eq) Et₃N are dissolved in 10 ml DCM and stirred over night at rt. The reaction is diluted with EtOAc and washed with aq. bicarbonate and brine. The organic layer is dried over sodium sulfate and the solvents are evaporated at reduced pressure. The residue is purified by chromatography on silica (flashmaster, hexane to hexane/EtOAc 1/4) to give the product.

MS (LC/MS): 472=[M+Na]⁺

¹H-NMR (400 MHz, CDCl₃): 7.26 (d, 1H), 7.13-7.09 (m, 3H), 6.15-6.14 (m, 2H), 6.07 (d, 1H), 6.01-5.90 (m, 2H), 5.29 (d, 1H), 5.20 (d, 1H), 5.12-5.08 (m, 1H), 5.07 (s, 1H), 4.7 (br s, 1H), 4.65-4.57 (m, 2H), 3.88 (s, 3H), 3.39 (d, 2H), 3.07-2.97 (m, 2H), 2.71-2.61 (m, 1H), 2.48-2.40 (m, 1H), 1.99-1.91 (m, 1H).

### b) (S*)-18-Methoxy-4-((2S*,4R*)-4-methyl-5-oxo-tetrahydro-furan-2-yl)-3,15,17-triaza-tricyclo[14.3.1.1*6,10*]henicosa-1(20),6(21),7,9,16,18-hexaen-2-one

To a solution of 0.24 g (0.53 mmol) 2-allylamino-N—[(S*)-2-(3-allyl-phenyl)-1-((2S*,4R*)-4-methyl-5-oxo-tetrahydro-furan-2-yl)-ethyl]-6-methoxy-isonicotinamide in 600 ml dry DCM under an argon atmosphere are added 23 mg (0.05 eq) benzylidene(1,3-dimesitylimidazolidin-2-ylidene)(tricyclohexylphosphine)ruthenium dichloride (CAS 246047-72-3) catalyst. The reaction is stirred at reflux temperature (bath temperature 60° C.) for 3 h. After a second addition of catalyst (23 mg) the reaction is heated over night at reflux. The solvent is evaporated and the residue is purified by chromatography on silica (flashmaster, hexane to EtOAc/hexanes 7/3), followed by crystallization from DCM/MeOH/ether/hexane. The resulting solid is hydrogenated with 88 mg Pd/C (10% Engelhard 4505) in 50 ml methanol/THF (1/1) at rt (1 atm H₂) during 7 h. After filtering through Celite the solvent is evaporated and the crude product is purified by chromatography on silica (flashmaster, DCM/MeOH 95/5). Recrystallization from DCM/MeOH/hexane gives the purified product.

MS (LC/MS): 446=[M+Na]⁺

¹H-NMR (400 MHz, CDCl₃): 7.29 (t, 1H), 7.18-7.11 (m, 3H), 6.24 (s, 1H), 5.82 (d, 1H), 5.26 (s, 1H), 4.89 (t, 1H), 4.82-4.78 (m, 1H), 4.68-4.62 (m, 1H), 3.80 (s, 3H), 3.3-3.1 (m, 3H), 3.04-2.97 (m, 1H), 2.87-2.60 (m, 4H), 2.14-2.02 (m, 2H), 1.84-1.74 (m, 1H), 1.63-1.51 (m, 1H), 1.44-1.34 (m, 1H), 1.31 (d, 3H).

### c) (2R*,4S*)—N-Butyl-4-hydroxy-4-((S*)-18-methoxy-2-oxo-3,15,17-triaza-tricyclo[14.3.1.1*6,10*]henicosa-1(20),6(21),7,9,16,18-hexaen-4-yl)-2-methyl-butyramide

A solution of 82 mg (0.19 mmol) (S*)-18-methoxy-4-((2S*,4R*)-4-methyl-5-oxo-tetrahydro-furan-2-yl)-3,15,17-triaza-tricyclo[14.3.1.1*6,10*]henicosa-1(20),6(21),7,9,16,18-hexaen-2-one in 7 ml n-butylamine is heated over night at reflux temperature. The amine is evaporated at reduced pressure and the residue is crystallized from DCM/hexane to give the product.

MS (LC/MS): 519=[M+Na]⁺

¹H-NMR (400 MHz, d6-DMSO): 7.95 (d, 1H), 7.67 (t, 1H), 7.43 (s, 1H), 7.17 (t, 1H), 7.05 (d, 1H), 7.01 (d, 1H), 6.61 (t, 1H), 5.89 (s, 1H), 5.70 (s, 1H), 4.83 (d, 1H), 4.11-4.04 (m, 1H), 3.69 (s, 3H), 3.60-3.55 (m, 1H), 3.36-3.30 (m, 1H), 3.08-2.96 (m, 3H), 2.91 (d, 1H), 2.75 (t, 1H), 2.7-2.65 (m, 2H), 1.95-1.7 (m, 3H), 1.45-1.1 (m, 8H), 1.05 (d, 3H), 0.88 (t, 3H).

### Example 3

### (2R,4S)—N-Butyl-4-hydroxy-4-((S)-19-methoxy-2-oxo-3,16,18-triaza-tricyclo[15.3.1.1*6,10*]docosa-1(21),6(22),7,9,17,19-hexaen-4-yl)-2-methyl-butyramide

The title compound is obtained by an analogous reaction sequence as for example 2, starting from f(S)-2-(3-allyl-phenyl)-1-((2S,4R)-4-methyl-5-oxo-tetrahydro-furan-2-yl)-ethyl]-carbamic acid tert-butyl ester (building block B3) and 2-but-3-enylamino-6-methoxy-isonicotinic acid (building block A3).

MS (LC/MS): 533=[M+Na]⁺

¹H-NMR (300 MHz, d6-DMSO): 8.04 (d, 1H), 7.67 (t, 1H), 7.15-7.10 (m, 2H), 7.02 (d, 1H), 6.96 (d, 1H), 6.53 (t, 1H), 6.06 (s, 1H), 6.02 (s, 1H), 4.79 (d, 1H), 3.95-3.85 (m, 1H), 3.72 (s, 3H), 3.55-3.45 (m, 1H), 3.3-3.2 (m, 1H), 3.09-2.96 (m, 3H), 2.8-2.75 (m, 2H), 2.6-2.35 (m, 2H), 1.85 (t, 1H), 1.75-1.2 (m, 1H), 1.6-1.15 (m, 11H), 1.03 (d, 3H), 0.86 (t, 3H).

### Example 4

### (2R,4S)—N-Butyl-4-hydroxy-2-methyl-4-((S)-19-methyl-2-oxo-3,16,18-triaza-tricyclo[15.3.1.1*6,10*]docosa-1(21),6(22),7,9,17,19-hexaen-4-yl)-butyramide

The title compound is obtained by an analogous reaction sequence as for example 2, starting from [(S)-2-(3-allyl-phenyl)-1-((2S,4R)-4-methyl-5-oxo-tetrahydro-furan-2-yl)-ethyl]-carbamic acid tert-butyl ester (building block B3) and 2-but-3-enylamino-6-methyl-isonicotinic acid hydrochloride (building block A1).

MS (LC/MS): 495=[M+H]⁺

¹H-NMR (300 MHz, d6-DMSO): 8.10 (d, 1H), 7.70 (t, 1H), 7.18-7.14 (m, 2H), 7.05 (d, 1H), 6.99 (d, 1H), 6.55-6.52 (m, 2H), 6.38 (s, 1H), 4.84 (d, 1H), 3.99-3.92 (m, 1H), 3.6-3.5 (m, 1H), 3.35-3.25 (m, 1H), 3.12-3.02 (m, 3H), 2.85-2.8 (m, 2H), 2.58-2.41 (m, 2H), 2.25 (s, 3H), 1.87 (t, 1H), 1.78-1.67 (m, 1H), 1.58-1.21 (m, 11H), 1.05 (d, 3H), 0.88 (t, 3H).

### Example 5

### (2R,4S)—N-Butyl-4-hydroxy-2-methyl-4-((S)-18-methyl-2-oxo-3,15,17-triaza-tricyclo[14.3.1.1*6,10*]henicosa-1 (20),6(21),7,9,16,18-hexaen-4-yl)-butyramide

### a) 2-Allylamino-N—[(S)-2-(3-allyl-phenyl)-1-((2S,4R)-4-methyl-5-oxo-tetrahydro-furan-2-yl)-ethyl]-6-methyl-isonicotinamide

A solution of 0.31 g (0.86 mmol) [(S)-2-(3-allyl-phenyl)-1-((2S,4R)-4-methyl-5-oxo-tetrahydro-furan-2-yl)-ethyl]-carbamic acid tert-butyl ester (building block B3) in 4 N HCl in dioxane is stirred at rt for 1 h. The solvent is evaporated and the residue is dried in vacuum. The obtained residue, 0.199 g (1.04 mmol, 1.2 eq) 2-allylamino-6-methyl-isonicotinic acid hydrochloride (building block A2), 0.14 g (1.0 mmol, 1.2 eq) HOBt, 0.248 g (1.29 mmol, 1.5 eq) EDC.HCl, and 0.60 ml (4.3 mmol, 5 eq) Et₃N are dissolved in 10 ml DCM and stirred over night at rt. The reaction is diluted with EtOAc and washed with aq. bicarbonate and brine. The organic layer is dried over sodium sulfate and the solvents are evaporated at reduced pressure. The residue is purified by chromatography on silica (flashmaster, hexane to hexane/EtOAc 3/7) to give the product.

MS (LC/MS): 456=[M+Na]⁺

¹H-NMR (400 MHz, CDCl₃): 7.27 (d, 1H), 7.14-7.11 (m, 3H), 6.58 (s, 1H), 6.53 (s, 1H), 6.33 (d, 1H), 6.02-5.89 (m, 2H), 5.33 (d, 1H), 5.24 (d, 1H), 5.10-5.00 (m, 1H), 4.70-4.60 (m, 3H), 3.97 (t, 2H), 3.39 (d, 2H), 3.12-3.01 (m, 2H), 2.72-2.61 (m, 1H), 2.08 (s, 3H), 2.02-1.95 (m, 1H), 1.99-1.91 (m, 1H), 1.30 (t, 3H).

### b) (S)-18-Methyl-4-((2S,4R)-4-methyl-5-oxo-tetrahydro-furan-2-yl)-3,15,17-triaza-tricyclo[14.3.1.1*6,10*]henicosa-1 (20),6(21),7,9,12,16,18-heptaen-2-one

To a solution of 269 mg (0.62 mmol) 2-allylamino-N—[(S)-2-(3-allyl-phenyl)-1-((2S,4R)-4-methyl-5-oxo-tetrahydro-furan-2-yl)-ethyl]-6-methyl-isonicotinamide in DCM is added 4 N HCl. The solvent is evaporated to give the hydrochloride salt. This salt is redissolved in 700 dry DCM and under an argon atmosphere 53 mg (0.1 eq) benzylidene(1,3-dimesitylimidazolidin-2-ylidene)(tricyclohexylphosphine)ruthenium dichloride (CAS 246047-72-3) catalyst are added. The reaction is stirred at reflux temperature (bath temperature 60° C.) for 1.5 h. The reaction mixture is washed with 2 N aq. sodium hydroxide, dried over sodium sulfate and the solvent is evaporated. The residue is purified by chromatography on silica (flashmaster, DCM to DCM/MeOH 9/1), followed by crystallization from DCM/MeOH/hexane to give the product.

MS (LC/MS): 428=[M+Na]⁺

¹H-NMR (400 MHz, d6-DMSO): 8.43 (d, 1H), 7.41 (s, 1H), 7.18-7.09 (m, 2H), 6.96 (d, 1H), 6.78 (t, 1H), 6.58 (s, 1H), 6.54 (s, 1H), 5.64-5.51 (m, 2H), 4.77-4.72 (m, 1H), 4.26-4.17 (m, 1H), 3.89 (br s, 2H), 3.34-3.33 (m, 1H), 3.21-3.16 (m, 1H), 2.95-2.80 (m, 2H), 2.63-2.55 (m, 1H), 2.47-2.40 (m, 1H), 2.23 (s, 3H), 2.04-1.97 (m, 1H), 1.16 (d, 3H).

### c) (2R,4S)—N-Butyl-4-hydroxy-2-methyl-4-((S)-18-methyl-2-oxo-3,15,17-triaza-tricyclo[14.3.1.1*6,10*]henicosa-1(20),6(21),7,9,12,16,18-heptaen-4-yl)-butyramide

A solution of 164 mg (0.40 mmol) (S)-18-methyl-4-((2S,4R)-4-methyl-5-oxo-tetrahydro-furan-2-yl)-3,15,17-triaza-tricyclo[14.3.1.1*6,10*]henicosa-1(20),6(21),7,9,12,16,18-heptaen-2-one in 20 ml n-butylamine is heated over night at reflux temperature. The amine is evaporated at reduced pressure and the residue is washed with ether and dried in vacuum to give the product.

MS (LC/MS): 479=[M+H]⁺

¹H-NMR (400 MHz, d6-DMSO): 8.12 (d, 1H), 7.67 (t, 1H), 7.40 (s, 1H), 7.11 (t, 1H), 7.02 (d, 1H), 6.93 (d, 1H), 6.73 (t, 1H), 6.58 (s, 1H), 6.52 (s, 1H), 5.73-5.65 (m, 1H), 5.55-5.50 (m, 1H), 4.86 (d, 1H), 3.95-3.85 (m, 2H), 3.58-3.50 (m, 1H), 3.41-3.33 (m, 1H), 3.21-3.16 (m, 1H), 3.04 (q, 2H), 2.82-2.78 (m, 2H), 2.7-2.65 (m, 1H), 2.23 (s, 3H), 1.83 (br t, 1H), 1.44-1.21 (m, 6H), 1.05 (d, 3H), 0.88 (t, 3H).

### d) (2R,4S)—N-Butyl-4-hydroxy-2-methyl-4-((S)-18-methyl-2-oxo-3,15,17-triaza-tricyclo[14.3.1.1*6,10*]henicosa-1(20),6(21),7,9,16,18-hexaen-4-yl)-butyramide

A solution of 183 mg (0.38 mmol) (2R,4S)—N-butyl-4-hydroxy-2-methyl-4-((S)-18-methyl-2-oxo-3,15,17-triaza-tricyclo[14.3.1.1*6,10*]henicosa-1(20),6(21),7,9,12,16,18-heptaen-4-yl)-butyramide in 60 ml DMF is hydrogenated with 122 mg Pd/C (10% Engelhard 4505) at rt (1 atm H₂) over night. After filtering through Celite the solvent is evaporated and the crude product is purified by chromatography on silica (flashmaster, DCM/MeOH 9/1). The solid is washed with hexane/DCM/MeOH and dried in vacuum to give the product.

MS (LC/MS): 481=[M+H]⁺

¹H-NMR (300 MHz, d6-DMSO): 7.99 (d, 1H), 7.68 (t, 1H), 7.43 (s, 1H), 7.18 (t, 1H), 7.06 (d, 1H), 7.01 (d, 1H), 6.59 (t, 1H), 6.39 (s, 1H), 5.97 (s, 1H), 4.84 (d, 1H), 4.15-4.05 (m, 1H), 3.65-3.55 (m, 1H), 3.11-2.88 (m, 4H), 2.77 (t, 1H), 2.7-2.6 (m, 2H), 2.59-2.55 (m, 1H), 2.20 (s, 3H), 1.95-1.7 (m, 3H), 1.42-1.11 (m, 8H), 1.05 (d, 3H), 0.89 (t, 3H).

### Example 6

### (S)-4-[(R)-1-Hydroxy-2-(3-isopropyl-benzylamino)-ethyl]-18-methoxy-15-methyl-3,15,17-triaza-tricyclo[14.3.1.1*6,10*]henicosa-1(20),6,8,10(21),16,18-hexaen-2-one

### a) N-[(1S,2R)-1-(3-Allyl-benzyl)-2-hydroxy-3-(3-isopropyl-benzylamino)-propyl]-2-(allyl-methyl-amino)-6-methoxy-isonicotinamide

A solution of 595 mg (1.01 mmol) {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) in 8.8 ml (35 eq) 4 N HCl in dioxane is stirred at rt for 1 h. The solvent is evaporated and the residue is redissolved in 35 ml DCM. 320 mg (1.24 mmol, 1.2 eq) 2-(allyl-methyl-amino)-6-methoxy-isonicotinic acid hydrochloride (building block A5), 297 mg (1.55 mmol, 1.5 eq) EDC.HCl, 190 mg (1.24 mmol, 1.2 eq) HOBT and 0.718 ml (5.16 mmol, 5 eq) Et₃N are added and the reaction is stirred at rt for 20 h. The mixture is diluted with EtOAc, washed with aq. bicarbonate and brine, and dried over sodium sulfate. The solvents are evaporated at reduced pressure and the residue is purified by chromatography on silica (flashmaster, hexane to hexane/EtOAc 3/2) to give the product.

MS (LC/MS): 691=[M+H]⁺

¹H-NMR (400 MHz, CDCl₃): 7.35-6.97 (m, 13H), 6.41 (d, 1H), 6.28-5.81 (m, 4H), 5.24-5.04 (m, 6H), 4.60 (d, 1H), 4.54 (d, 1H), 4.37-4.28 (m, 1H), 4.18 (d, 2H), 3.95 (d, 1H), 3.89 (s, 3H), 3.48 (d, 2H), 3.37 (d, 2H), 3.03 (s, 3H), 3.05-2.98 (m, 2H), 2.91-2.77 (m, 1H), 1.20 (d, 6H).

### b) (S)-4-[(R)-1-Hydroxy-2-(3-isopropyl-benzylamino)-ethyl]-18-methoxy-15-methyl-3,15,17-triaza-tricyclo[14.3.1.1*6,10*]henicosa-1(20),6,8,10(21),16,18-hexaen-2-one

To a solution of 500 mg (0.72 mmol) N-[(1S,2R)-1-(3-allyl-benzyl)-2-hydroxy-3-(3-isopropyl-benzylamino)-propyl]-2-(allyl-methyl-amino)-6-methoxy-isonicotinamide in DCM are added 0.7 ml (4 eq) 4 N HCl in dioxane. The solvents are evaporated and the obtained hydrochloride salt is dried in vacuum. This salt is redissolved in 20 ml dry DCM and the solution is added dropwise over 30 min under an argon atmosphere, at reflux temperature (bath temperature 60° C.) to the solution of 61 mg (0.1 eq) benzylidene(1,3-dimesitylimidazolidin-2-ylidene) (tricyclohexylphosphine)ruthenium dichloride (CAS 246047-72-3) catalyst in 980 ml dry DCM. The reaction is stirred at reflux temperature for another 3 h. 0.4 ml Butylvinylether are added, after 10 min the reaction mixture is cooled to rt and washed with aq, bicarbonate and brine, dried over sodium sulfate and the solvent is evaporated. The residue is purified by chromatography on silica (flashmaster, hexane to hexane/EtOAc 1/1) followed by crystallization from DCM/ether/hexane. The obtained olefin is hydrogenated with 80 mg Pd/C (5% Degussa E101 N/D) in 120 ml MeOH/THF (1/1) at rt (1 atm H₂) during 2.35 h. After filtering through glasswool the solvent is evaporated and the crude product is purified by chromatography on silica plates (DCM/MeOH 9/1 with 1% aq. ammonia). Recrystallization from DCM/ether/hexane gives the product (the product contains traces of the ring-opened side product N-[(1S,2R)-1-(3-Butyl-benzyl)-2-hydroxy-3-(3-isopropyl-benzylamino)-propyl]-2-methoxy-6-methylamino-isonicotinamide).

MS (LC/MS): 531=[M+H]⁺

¹H-NMR (300 MHz, CDCl₃): 7.34-7.16 (m, 8H), 6.25 (s, 1H), 5.85 (d, 1H), 5.75 (s, 1H), 4.31-4.23 (m, 1H), 3.88 (s, 3H), 3.62-3.75 (m, 1H), 3.44-3.29 (m, 2H), 3.16 (s, 3H), 3.12-3.08 (m, 2H), 2.97-2.90 (m, 2H), 2.84-2.75 (m, 4H), 2.05-1.86 (m, 2H), 1.60-1.43 (m, 2H), 1.40-1.30 (m, 1H), 1.29 (d, 6H).

### Example 7

### (S)-4-[(R)-1-Hydroxy-2-(3-isopropyl-benzylamino)-ethyl]-18-methyl-3,17-diaza-tricyclo[14.3.1.1*6,10*]henicosa-1(20),6,8,10(21),16,18-hexaen-2-one

### a) {(2R,3S)-4-(3-Allyl-phenyl)-3-[((2-but-3-enyl-6-methyl-pyridine-4-carbonyl)-amino]-2-hydroxy-butyl}-(3-isopropyl-benzyl)-carbamic acid benzyl ester

A solution of 0.42 g (0.72 mmol) {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) in 10 ml 4 N HCl in dioxane is stirred at rt for 1 h. The solvent is evaporated and the residue is redissolved in 15 ml DCM. To the solution 244 mg (1.07 mmol, 1.5 eq) 2-but-3-enyl-6-methyl-isonicotinic acid hydrochloride (building block A6), 116 mg (0.86 mmol, 1.2 eq) HOBt, 206 mg (1.07 mmol, 1.5 eq) EDC.HCl and 0.50 ml (3.6 mmol, 5 eq) Et₃N are added and the reaction stirred over night at rt. The reaction is diluted with EtOAc and washed with aq. bicarbonate and brine. The organic layer is dried over sodium sulfate and the solvents are evaporated at reduced pressure. The residue is purified by chromatography on silica (flashmaster, DCM to DCM/MeOH 9/1) followed by chromatography on silica (flashmaster, hexane to hexane/EtOAc 3/7) to give the product.

MS (LC/MS): 660=[M+H]⁺

¹H-NMR (300 MHz, d6-DMSO): 7.35-6.97 (m, 15H), 6.55 (d, 1H), 5.96-5.84 (m, 2H), 5.27 (d, 1H), 5.22 (d, 1H), 5.10-5.00 (m, 4H), 4.58 (br s, 2H), 4.38 (br s, 1H), 4.29 (br s, 1H), 3.99 (br s, 1H), 3.53-3.42 (m, 2H), 3.36 (d, 2H), 3.00-2.80 (m, 5H), 2.62-2.47 (m, 5H), 1.21 (d, 6H).

### b) (S)-4-[(R)-1-Hydroxy-2-(3-isopropyl-benzylamino)-ethyl]-18-methyl-3,17-diaza-tricyclo[14.3.1.1*6,10*]henicosa-1(20),6,8,10(21),16,18-hexaen-2-one

To a solution of 370 mg (0.56 mmol) {(2R,3S)-4-(3-Allyl-phenyl)-3-[(2-but-3-enyl-6-methyl-pyridine-4-carbonyl)-amino]-2-hydroxy-butyl}-(3-isopropyl-benzyl)-carbamic acid benzyl ester in DCM are added 0.7 ml (5 eq) 4 N HCl in dioxane and the mixture is stirred for 1 h. The solvents are evaporated and the obtained hydrochloride salt is dried in vacuum. This salt is redissolved in 700 ml dry DCM under an argon atmosphere and 48 mg (0.1 eq) benzylidene-(1,3-dimesitylimidazolidin-2-ylidene)(tricyclohexylphosphine)ruthenium dichloride (CAS 246047-72-3) catalyst are added. The reaction is stirred at reflux temperature (bath temperature 60° C.) for 3 days. Additional 80 mg catalyst are added after 4.5 h and 30 mg after 1 day. 0.9 ml Butylvinylether are added thereafter and the reaction mixture is washed with 2 N aq. sodium hydroxide, dried over sodium sulfate and the solvent is evaporated. The residue is purified by chromatography on silica (flashmaster, hexane to hexane/EtOAc 1/9). The obtained olefin is hydrogenated with 54 mg Pd/C (5% Degussa E101 N/D) in 40 ml methanol at rt (1 atm H₂) during 9 h. After filtering through celite the solvent is evaporated and the crude product is purified by chromatography on silica plates (DCM/MeOH 95/5 with 1% aq. ammonia). Recrystallization from DCM/MeOH/ether/hexane gives the purified product.

MS (LC/MS): 500=[M+H]⁺

¹H-NMR (400 MHz, d6-DMSO): 8.20 (d, 1H), 7.30 (s, 1H), 7.24-6.97 (m, 8H), 6.85 (d, 1H), 4.98 (br s, 1H), 4.22-4.13 (m, 1H), 3.72 (s, 2H), 3.63 (br s, 1H), 3.17 (dd, 1H), 2.88-2.78 (m, 3H), 2.75-2.40 (m, 6H), 2.40 (s, 3H), 2.0-1.45 (m, 4H), 1.18 (d, 6H), 1.1-1.0 (m, 1H), 0.9-0.8 (m, 1H).

### Example 8

### (S)₄-[(R)-1-Hydroxy-2-(3-isopropylbenzylamino)-ethyl]-19-trifluoromethyl-11-oxa-3,16-diaza-tricyclo[15.3.1.1*6,10*]docosa-1(21),6,8,10(22),17,19-hexaen-2-one

The title compound is obtained by an analogous reaction sequence as for example 6, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-methoxy-benzoic acid (building block A8).

MS (ES+): 570=[M+H]⁺

¹H-NMR (400 MHz, CDCl₃): 7.40 (s, 1H), 7.37-7.25 (m, 5H), 7.22-7.15 (m, 3H), 7.04 (d, 1H), 6.88 (s, 2H), 6.83 (d, 1H), 6.77 (s, 1H), 6.04 (d, 1H), 4.30-4.19 (m, 2H), 4.19-4.10 (m, 2H), 3.90-3.80 (m, 2H), 3.70-3.60 (m, 4H), 3.55-3.45 (m, 1H), 3.38-3.20 (m, 3H), 3.00-2.85 (m, 2H), 2.81-2.70 (m, 2H), 1.80-1.60 (m, 2H), 1.25 (d, 6H).

### Example 9

### (S)₄-[(R)-1-Hyroxy-2-(3-isopropylbenzylamino)-ethyl]-19-methoxy-11,16-dioxa-3-aza-tricyclo[15.3.1.1*6,10*]docosa-1(21),6,8,10(22),17,19-hexaen-2-one

The title compound is obtained by an analogous reaction sequence as for example 6, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-trifluoromethyl-benzoic acid (building block A7).

Rf: (EtOAc/hexane/NH₃ 50:49:1): 0.10

MS (ES+): 533=[M+H]⁺.

### Example 10

### (S)-4-[(R) —-Hydroxy-2-(3-isopropyl-benzylamino)-ethyl]-18-methoxy-3,15,17-triaza-tricyclo[14.3.1.1*6,10*]henicosa-1(19),6,8,10(21),16(20),17-hexaen-2-one

The title compound is obtained by an analogous reaction sequence as for example 6, starting from {(1S,2R)-1-(3-Allyl-benzyl)-3-[benzyloxycarbonyl-(3-isopropyl-benzyl)-amino]-2-hydroxy-propyl}-carbamic acid tert-butyl ester (building block C5) and 2-Allylamino-6-methoxy-isonicotinic acid (building block A4).

m.p. (of HCl-salt): 140-145° C.

Rf: (DCM/methanol/NH₃=90/9/1): 0.46

MS (ES⁺): 517.4=[MH]⁺.

### Example 11

### (S)-4-[(R)-1-Hydroxy-2-(3-isopropyl-benzylamino)-ethyl]-18-methyl-3,15,17-triaza-tricyclo[14.3.1.1*6,10*]henicosa-1(19),6,8,10(21),16(20),17-hexaen-2-one

The title compound is obtained by an analogous reaction sequence as for example 6, starting from {(1S,2R)-1-(3-Allyl-benzyl)-3-[benzyloxycarbonyl-(3-isopropyl-benzyl)-amino]-2-hydroxy-propyl}-carbamic acid tert-butyl ester (building block C5) and 2-Allylamino-6-methyl-isonicotinic acid hydrochloride (building block A2).

Rf: (DCM/methanol=90/10): 0.23

MS (ES+): 501=[MH]⁺

¹H-NMR (400 MHz, d6-DMSO): 8.06 (d, 1H), 7.34 (s, 1H), 7.26-7.20 (m, 2H), 7.19-7.10 (m, 3H), 7.04-6.96 (m, 2H), 6.57 (t, 1H), 6.33 (s, 1H), 5.85 (s, 1H), 5.15 (br, 1H), 4.08-3.98 (m, 1H), 3.83 (s, 2H), 3.67-3.60 (m, 1H), 3.12 (dd, 1H), 3.04-2.92 (m, 1H), 2.90-2.82 (m, 1H), 2.78-2.60 (m, 6H), 2.18 (s, 3H), 1.98-1.86 (m, 1H), 1.79-1.69 (m, 1H), 1.42-1.28 (m, 1H), 1.25-1.12 (m, 2H), 1.20 (d, 6H).

### Example 12

### (S)-4-[(R)-1-Hydroxy-2-(3-isopropyl-benzylamino)-ethyl]-11-oxa-3,16-diaza-tricyclo[15.3.1.1*6,10*]docosa-1(21),6,8,10(22),17,19-hexaen-2-one hydrochloride

### a) Allyl-(3-{(1S,2R)-1-(3-allyloxy-benzyl)-3-[benzyloxycarbonyl-(3-isopropyl-benzyl)-amino]-2-hydroxy-propylcarbamoyl}-phenyl)-carbamic acid tert-butyl ester

To the solution of 0.454 g (0.84 mmol, 1 eq) {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 0.280 g (1.0 mmol, 1.2 eq) 3-(allyl-tert-butoxycarbonyl-amino)-benzoic acid (building block A9) in 15 ml DCM are added 0.137 g (1.0 mmol, 1.2 eq) HOBT, 0.242 g (1.26 mmol, 1.5 eq) EDC.HCl, and 0.59 ml (4.2 mmol, 5 eq) triethylamine. The reaction mixture is stirred at rt over night. The mixture is diluted with EtOAc, washed with aqueous sodium bicarbonate and brine, dried over sodium sulfate, and the solvents are evaporated at reduced pressure. The residue is purified by chromatography on silica (flashmaster, hexane to hexane/EtOAc 3/2) to give the product.

MS (LC/MS): 762=[M+H]⁺

¹H-NMR (400 MHz, CDCl₃): 7.58 (s, 1H), 7.4-7.32 (m, 8H), 7.23-7.19 (m, 2H), 7.13 (d, 1H), 7.02 (s, 1H), 6.98 (d, 1H), 6.84-6.79 (m, 3H), 6.50 (d, 1H), 6.09-6.00 (m, 1H), 5.96-5.87 (m, 1H), 5.40 (d, 1H), 5.29-5.13 (m, 6H), 4.64-4.50 (m, 4H), 4.38 (br s, 1H), 4.26 (d, 2H), 3.54-3.46 (m, 2H), 3.00 (d, 2H), 2.9-2.79 (m, 2H), 1.47 n (s, 9H), 1.20 (d, 6H).

### b) (S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaene-16-carboxylic acid tert-butyl ester

A solution of 0.49 g (0.64 mmol) allyl-(3-{(1S,2R)-1-(3-allyloxy-benzyl)-3-[benzyloxycarbonyl-(3-isopropyl-benzyl)-amino]-2-hydroxy-propylcarbamoyl}-phenyl)-carbamic acid tert-butyl ester and 55 mg (0.1 eq) benzylidene(1,3-dimesitylimidazolidin-2-ylidene)(tricyclohexylphosphine) ruthenium dichloride (CAS 246047-72-3) in 20 ml dry DCM under an argon atmosphere is heated to reflux temperature (bath temperature 60° C.). After 3 h 1.0 ml butylvinylether is added, after 10 min the reaction mixture is cooled to rt, some charcoal is added and the mixture is filtered through glasswool. The major part of the solvents are evaporated at reduced pressure and the remaining concentrated mixture is purified by chromatography on silica (flashmaster, hexane to hexane/EtOAc 2/3). The obtained olefin is hydrogenated with 120 mg Pd/C (5% Degussa E101 N/D) in 120 ml MeOH/THF (20/1) at rt (1 atm H₂) during 2 h. After filtering through celite the solvent is evaporated and the residue is purified by chromatography on silica (flashmaster, DCM to DCM/MeOH 9/1) to give the product.

MS (LC/MS): 602=[M+H]⁺

¹H-NMR (400 MHz, CDCl₃): 7.69 (d, 1H), 7.44-7.15 (m, 7H), 7.05 (s, 1H), 6.97 (d, 1H), 6.79 (d, 1H), 6.73 (s, 1H), 6.10 (br d, 1H), 4.27-4.10 (m, 3H), 3.89-3.82 (m, 3H), 3.70-3.61 (m, 2H), 3.12 (dd, 1H), 3.01-2.89 (m, 2H), 2.83 (d, 2H), 1.84-1.68 (m, 4H), 1.45 (s, 9H), 1.27 (d, 6H).

### c) (S)-4-[(R)-1-Hydroxy-2-(3-isopropyl-benzylamino)-ethyl]-11-oxa-3,16-diaza-tricyclo[15.3.1.1*6,10*]docosa-1 (21),6,8,10(22),17,19-hexaen-2-one hydrochloride

The solution of 0.172 g (0.29 mmol) (S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaene-16-carboxylic acid tert-butyl ester in 2.5 ml of 4 M HCl in dioxane is stirred over night at rt. The solvent is evaporated at reduced pressure. The residue titurated in diethylether and filtered off to give after drying in vacuum the product in the form of the hydrochloride salt.

MS (LC/MS): 502=[M+H]⁺

¹H-NMR (400 MHz, d6-DMSO): 9.26 (br s, 1H), 8.95 (br s, 1H), 8.29 (br d, 1H), 7.45 (s, 1H), 7.35-7.28 (m, 3H), 7.19-7.11 (m, 3H), 6.92 (br s, 2H), 6.80 (d, 1H), 6.73 (d, 1H), 4.25-4.15 (m, 3H), 3.92-3.82 (m, 3H), 3.55-3.48 (m, 2H), 3.15-3.05 (m, 3H), 2.92-2.83 (m, 2H), 2.68 (t, 1H), 3.75-3.62 (m, 4H), 1.5-1.4 (m, 1H), 1.21 (d, 6H).

### Example 13

### (S)-4-[(R)-1-Hydroxy-2-(3-isopropyl-benzylamino)-ethyl]-18-methoxy-3,17-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 2-but-3-enyl-6-methoxy-isonicotinic acid (building block A10).

MS (LC/MS): 516=[M+H]⁺

¹H-NMR (400 MHz, d6-DMSO, 362K): 7.81 (d, 1H), 7.25-7.0 (m, 7H), 6.87 (d, 1H), 6.74 (s, 1H), 6.67 (s, 1H), 4.25-4.15 (m, 1H), 3.83 (s, 3H), 3.78 (s, 2H), 3.75-3.70 (m, 1H), 3.15 (dd, 1H), 2.93-2.83 (m, 1H), 2.83-2.60 (m, 6H), 2.52-2.43 (m, 1H), 1.93-1.75 (m, 2H), 1.68-1.53 (m, 2H), 1.22 (d, 6H), 1.22-1.08 (m, 1H), 1.05-0.92 (m, 1H).

### Example 14

### (S)-4-[(R)-1-Hydroxy-2-(3-isopropyl-benzylamino)-ethyl]-18-methoxy-3,15-diaza-tricyclo[14.3.1.1*6,10*]heicosa-1(20),6,8,10(21),16,18-hexaen-2-one

The title compound is obtained by an analogous reaction sequence as for example 12, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-(allyl-tert-butoxycarbonyl-amino)-5-methoxy-benzoic acid (building block A11).

MS (LC/MS): 516=[M+H]⁺

¹H-NMR (400 MHz, CDCl₃): 7.34-7.14 (m, 9H), 6.70 (s, 1H), 6.18 (s, 1H), 6.08 (s, 1H), 5.96 (d, 1H), 4.36-4.28 (m, 1H), 4.10 (br s, 1H), 3.96-3.86 (m, 2H), 3.79 (s, 3H), 3.65-3.61 (m, 1H), 3.30-3.07 (m, 4H), 2.97-2.87 (m, 1H), 2.86-2.73 (m, 4H), 1.99-1.80 (m, 2H), 1.62-1.45 (m, 2H), 1.28 (d, 6H).

### Example 15

### (S)-4-[(R)-1-Hydroxy-2-(3-isopropyl-benzylamino)-ethyl]-3,17-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 2-but-3-enyl-isonicotinic acid (building block A12).

MS (LC/MS): 486=[M+H]⁺

¹H-NMR (400 MHz, CDCl₃): 8.61 (d, 1H), 7.34 (d, 1H), 7.34-7.16 (m, 8H), 6.70 (s, 1H), 7.01 (d, 1H), 6.92 (s, 1H), 6.33 (br s, 1H), 4.37-4.30 (m, 1H), 3.98 (d, 1H), 3.92 (d, 1H), 3.86-3.80 (m, 1H), 3.19 (dd, 1H), 3.06-2.82 (m, 7H), 2.67-2.60 (m, 1H), 1.90-1.70 (m, 4H), 1.28 (d, 6H), 1.26-1.07 (m, 2H).

### Example 16

### (S)-4-[(R)-1-Hydroxy-2-(3-isopropyl-benzylamino)-ethyl]-3,15-diaza-tricyclo[14.3.1.1*6,10*]henicosa-1(20),6,8,10(21),16,18-hexaen-2-one

The title compound is obtained by an analogous reaction sequence as for example 12, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-(allyl-tert-butoxycarbonyl-amino)-benzoic acid (building block A9).

MS (LC/MS): 486=[M+H]⁺

¹H-NMR (400 MHz, d6-DMSO): 9.24 (br s, 1H), 8.95 (br s, 1H), 8.23 (d, 1H), 7.46-7.28 (m, 5H), 7.18 (t, 1H), 7.11-701 (m, 3H), 6.8-6.75 (m, 2H), 6.49 (br s, 1H), 0.20-4.18 (m, 2H), 4.07-3.99 (m, 1H), 3.86 (br t, 1H), 3.43-3.35 (m, 1H), 3.22 (dd, 1H), 3.12-2.85 (m, 4H), 2.76-2.85 (m, 3H), 1.95-1.75 (, 2H), 1.45-1.35 (m, 1H), 1.22 (d, 6H), 1.28-1.1 (m, 2H).

### Example 17

### (S)-4-[(R)-1-Hydroxy-2-(3-isopropyl-benzylamino)-ethyl]-11-oxa-3,18-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 2-but-3-enyl-isonicotinic acid (building block A12).

MS (LC/MS): 502=[M+H]⁺

¹H-NMR (400 MHz, CDCl₃): 8.66 (d, 1H), 7.51 (d, 1H), 7.33-7.17 (m, 6H), 7.10 (s, 1H), 6.98 (d, 1H), 6.82 (d, 1H), 6.70 (s, 1H), 6.44 (d, 1H), 4.32-4.24 (m, 1H), 4.10-4.07 (m, 2H), 3.93 (d, 1H), 3.86 (d, 1H), 3.70-3.65 (m, 1H), 3.19 (dd, 1H), 2.99-2.79 (m, 6H), 1.98-1.79 (m, 5H), 1.50-1.43 (m, 1H), 1.26 (d, 6H).

### Example 18

### (S)-4-[(R)-1-Hydroxy-2-(3-isopropyl-benzylamino)-ethyl]-15-methyl-3,15-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-(allyl-methyl-amino)-benzoic acid (building block A14).

MS (LC/MS): 500=[M+H]⁺

¹H-NMR (400 MHz, d6-DMSO): 7.96 (d, 1H), 7.40 (s, 1H), 7.23-7.09 (m, 6H), 7.06 (d, 1H), 7.00 (d, 1H), 6.72 (d, 1H), 6.61 (br d, 1H), 6.52 (s, 1H), 4.09 (br s, 1H), 4.14-4.06 (m, 1H), 3.86-3.76 (m, 1H), 3.72 (s, 2H), 3.64 (br s, 1H), 3.11 (dd, 1H), 3.05-2.97 (m, 1H), 2.89 (s, 3H), 2.89-2.83 (m, 1H), 2.74-2.57 (m, 6H), 1.98-1.89 (m, 1H), 1.81-1.72 (m, 1H), 1.32-1.23 (m, 2H), 1.20 (d, 6H).

### Example 19

### (S)-4-{(R)-1-Hydroxy-2-[(4-isopropyl-pyridin-2-ylmethyl)-amino]-ethyl}-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(4-isopropyl-pyridin-2-ylmethyl)-amino]-2-hydroxy-propyl}-carbamic acid tert-butyl ester (building block C7) and 3-allyloxy-benzoic acid (building block A15).

MS (LC/MS): 504=[M+H]⁺

¹H-NMR (400 MHz, C₂D₂Cl₄, 362K): 8.53 (d, 1H), 7.47 (d, 1H), 7.38 (t, 2H), 7.27-7.22 (m, 2H), 7.12 (d, 1H), 7.07 (d, 1H), 6.99 (s, 1H), 6.88 (d, 2H), 6.26 (br d, 1H), 4.40-4.12 (m, 7H), 3.87 (br s, 1H), 3.26 (dd, 1H), 3.14-2.97 (m, 4H), 2.13-2.0 (m, 4H), 1.36 (d, 6H).

### Example 20

### (S)-4-(R)-1-Hydroxy-2-[(4-isopropyl-pyridin-2-ylmethyl)-amino]-ethyl}-1-oxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(4-isopropyl-pyridin-2-ylmethyl)-amino]-2-hydroxy-propyl}-carbamic acid tert-butyl ester (building block C7) and 3-but-3-enyl-benzoic acid (building block A16).

MS (LC/MS): 502=[M+H]

¹H-NMR (400 MHz, C₂D₂Cl₄, 362K): 8.48 (d, 1H), 7.72 (d, 1H), 7.43-7.33 (m, 3H), 7.31 (s, 1H), 7.25 (s, 1H), 7.16 (d, 1H), 7.04 (d, 1H), 6.38-6.35 (m, 2H), 6.42 (br d, 1H), 4.40-4.32 (m, 1H), 4.20-4.13 (m, 4H), 3.87 (br s, 1H), 3.27 (dd, 1H), 3.16-2.92 (m, 5H), 2.30-2.20 (m, 2H), 1.93-1.75 (m, 4H), 1.58-1.50 (m, 2H), 1.35 (d, 6H).

### Example 21

### (S)-4-{(R)-1-Hydroxy-2-(4-isopropyl-pyridin-2-ylmethyl)-amino]-ethyl}-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(4-isopropyl-pyridin-2-ylmethyl)-amino]-2-hydroxy-propyl}-carbamic acid tert-butyl ester (building block C7) and 3-(allyl-tert-butoxycarbonyl-amino)-benzoic acid (building block A9).

MS (LC/MS): 503=[M+H]⁺

¹H-NMR (400 MHz, C₂D₂Cl₄, 362K): 8.50 (d, 1H), 7.47 (t, 1H), 7.25-7.15 (m, 4H), 7.08 (d, 1H), 7.02 (s, 1H), 6.89 (d, 1H), 6.26 (d, 1H), 6.18 (s, 1H), 6.27 (br d, 1H), 4.40-4.33 (m, 1H), 4.27-4.18 (m, 2H), 4.13 (s, 2H), 3.78-3.70 (m, 1H), 3.40-3.26 (m, 3H), 3.07-2.94 (m, 4H), 1.98-1.78 (m, 4H), 1.36 (d, 6H).

### Example 22

### (S)-4-[(R)-1-Hydroxy-2-(3-isopropyl-benzylamino)-ethyl]-19-methyl-11-oxa-3,16,18-triaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-alyloxy-benzyl)-3-[benzyloxycarbonyl-(3-isopropylbenzyl)-amino]-2-hydroxy-propyl}-carbamic acid tert-butyl ester (building block C6) and 2-allylamino-6-methyl-isonicotinic acid hydrochloride (building block A2).

MS (LC/MS): 517=[M+H]⁺

¹H-NMR (400 MHz, d6-DMSO): 8.13 (d, 1H), 7.24-7.10 (m, 5H), 7.03 (s, 1H), 6.81 (d, 2H), 6.77 (d, 1H), 6.56 (t, 1H), 6.32 (s, 1H), 6.16 (s, 1H), 5.00 (br s, 1H), 4.28-4.21 (m, 1H), 3.96-3.83 (m, 2H), 3.72 (s, 2H), 3.75 (br s, 1H), 3.02 (br d, 2H), 2.89-2.82 (m, 1H), 2.67-2.60 (m, 2H), 2.56-2.45 (m, 2H), 2.22 (s, 3H), 1.8-1.6 (m, 3H), 1.55-1.4 (m, 1H), 1.19 (d, 6H).

### Example 23

### Propane-1-sulfonic acid {(S)₄-[(R)-1-hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-methyl-amide

### a) (Z)-(S)-4-{(R)-2-[Benzyloxycarbonyl-(3-isopropyl-benzyl)-amino]-1-hydroxy-ethyl}-19-nitro-2-oxo-11-oxa-3,16-diaza-tricyclo[15.3.1.1′6,10′]docosa-1(21),6,8,10(22),13,17,19-heptaene-16-carboxylic acid benzyl ester

The title compound is obtained by an analogous reaction sequence as for example 7 via an amide coupling and a metathesis reaction, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-nitro-benzoic acid (building block A19).

MS: 813 (M+1), 811 (M−1)

HPLC (Nucleosil C18HD, 20-100% AcCN): retention time=6.83 min

### b) (S)-[9-Amino-4-{(R)-2-[benzyloxycarbonyl-(3-isopropyl-benzyl)-amino]-1-hydroxy-ethyl}-2-oxo-11-oxa-3,16-diaza-tricyclo[15.3.1.1′6,10′]docosa-1(21),6,8,10(22),13,17,19-hexaene-16-carboxylic acid benzyl ester

(Z)-(S)-4-{(R)-2-[Benzyloxycarbonyl-(3-isopropyl-benzyl)-amino]-1-hydroxy-ethyl}-19-nitro-2-oxo-11-oxa-3,16-diaza-tricyclo[15.3.1.1′6,10′]docosa-1(21),6,8,10(22),13,17,19-heptaene-16-carboxylic acid benzyl ester (85 mg, 104 μmol, 1 eq) is dissolved in MeOH (5 ml) and hydrogenated using PtO₂ (10 mg, Engelhard 7018) and 1 bar of hydrogen. The reaction mixture is filtered and concentrated to give the product.

MS: 785 (M+1), 783 (M−1)

HPLC (Nucleosil C18HD, 20-100% AcCN): retention time=6.08 min

### c) (S)₄-{(R)-2-[Benzyloxycarbonyl-(3-isopropyl-benzyl)-amino]-1-hydroxy-ethyl}-2-oxo 19-(propane-1-sulfonylamino)-11-oxa-3,16-diaza-tricyclo[15.3.1.1′6,10′]docosa-1(21),6,8,10(22),13,17,19-hexaene-16-carboxylic acid benzyl ester

(S)-19-Amino-4-{(R)-2-[benzyloxycarbonyl-(3-isopropyl-benzyl)-amino]-1-hydroxy-ethyl}-2-oxo-11-oxa-3,16-diaza-tricyclo[15.3.1.1′6,10′]docosa-1(21),6,8,10(22),13,17,19-hexaene-16-carboxylic acid benzyl ester (85 mg, 107 μmol, 1 eq) is dissolved in DCM (1 ml) at 0° C. Pyridine (31 μl, 429 μmol, 4.0 eq) and propanesulfonylchloride (54 μl, 472 μmol, 4.4 eq) are added and the reaction is stirred for 8.5 h. The reaction mixture is diluted with 1N aq HCl and ethyl acetate. The organic layer is washed with brine, dried over Na₂SO₄, filtered and concentrated. The residue is purified by column chromatography using EtOAc/hexane=1/1 to give the product in the form of white crystals.

MS: 892 (M+1), 890 (M−1)

HPLC (Nucleosil C18HD, 20-100% AcCN): retention time=6.54 min

### d) (S)-4-{(R)-2-[Benzyloxycarbonyl-(3-isopropyl-benzyl)-amino]-hydroxy-ethyl}-2-oxo 19-[methyl-(propane-1-sulfonyl)amino]-11-oxa-3,16-diaza-tricyclo[15.3.1.1′6,10′]docosa-1(21),6,8,10(22),13,17,19-hexaene-16-carboxylic acid benzyl ester

(S)-4-{(R)-2-[Benzyloxycarbonyl-(3-isopropyl-benzyl)-amino]-1-hydroxy-ethyl}-2-oxo 19-(propane-1-sulfonylamino)-1-oxa-3,16-diaza-tricyclo[15.3.1.1′6,10′]docosa-1(21),6,8,-10(22),13,17,19-hexaene-16-carboxylic acid benzyl ester (40 mg, 44 μmol, 1 eq) is dissolved in acetonitrile (2 ml). K₂CO₃ (17.4 mg, 124 μmol, 2.8 eq) and MeI (14 μl, 220 μmol, 5 eq) are added and the reaction is stirred for 12 h. The reaction mixture is diluted with EtOAc, washed with brine, dried over Na₂SO₄, filtered and concentrated to give the product.

MS: 905 (M+1), 903 (M−1)

HPLC (Nucleosil C18HD, 20-100% AcCN): retention time=6.77 min

### e) Propane-1-sulfonic acid {(S)-4-[(R)-1-hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-methyl-amide

The title compound is obtained by an analogous hydrogenation reaction as for the last step in example 7, starting from (S)-4-{(R)-2-[benzyloxycarbonyl-(3-isopropyl-benzyl)-amino]-1-hydroxy-ethyl}-2-oxo 19-[methyl-(propane-1-sulfonyl)amino]-1-oxa-3,16-diaza-tricyclo[15.3.1.1′6,10′]docosa-1(21),6,8,10(22),13,17,19-hexaene-16-carboxylic acid benzyl ester.

HPLC (Nucleosil C18HD, 20-100% AcCN): retention time=4.26 min

MS: 637 (M+1), 635 (M−1)

¹H-NMR (400 MHz, CDCl₃): 7.38-7.22 (m, 2H), 7.20-7.15 (m, 3H), 7.10 (s, 1H), 7.02 (d, 1H), 6.90 (s, 1H), 6.87-6.80 (m, 2H), 6.53 (s, 1H), 6.10 (d, 1H), 4.30-4.00 (m, 5H), 3.80 (s, 2H), 3.55-3.48 (m, 1H), 3.34 (s, 3H), 3.34-3.20 (m, 3H), 3.00-2.83 (m, 4H), 2.83-2.75 (m, 2H), 2.00-1.65 (m, 6H), 1.24 (d, 6H), 1.03 (t, 3H).

### Example 24

### (S)-4-[(R)-1-Hydroxy-2-(3-isopropyl-benzylamino)-ethyl]-19-oxazol-2-yl-11-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-oxazol-2-yl-benzoic acid (building block A17).

HPLC (Nucleosil C18HD, 20-100% AcCN): retention time=4.00 min

MS: 569 (M+1), 567 (M−1)

¹H-NMR (400 MHz, CDCl₃): 7.82 (s, 1H), 7.77 (s, 1H), 7.40 (s, 1H), 7.38-7.19 (m, 5H), 7.18 (d, 1H), 7.10 (d, 1H), 6.92 (s, 1H), 6.80 (d, 1H), 6.70 (s, 1H), 6.08 (d, 1H), 4.30-4.00 (m, 4H), 3.80 (s, 2H), 3.58-3.50 (m, 1H), 3.38-3.28 (m, 2H), 3.02 (dd, 1H), 3.00-2.74 (m, 4H), 2.00-1.72 (m, 4H), 1.22 (d, 6H).

### Example 25

### N—((S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-methanesulfonamide

The title compound is obtained by an analogous hydrogenation reaction as the last step for example 7, starting from (S)-4-{(R)-2-[benzyloxycarbonyl-(3-isopropyl-benzyl)-amino]-1-hydroxy-ethyl}-19-methanesulfonylamino-2-oxo-11-oxa-3,16-diaza-tricyclo[15.3.1.1′6,10′]docosa-1(21),6,8,10(22),13,17,19-hexaene-16-carboxylic acid benzyl ester (see example 29a).

HPLC (Nucleosil C18HD, 20-100% AcCN): retention time=3.69 min

MS: 595 (M+1), 593 (M−1)

### Example 26

### Dimethyl-carbamic acid (S)-4-[(R)-1-hydroxy-2-(3-isopropyl-benzylamino)-ethyl]-2-oxo-11,16-dioxa-3-aza-tricyclo[15.3.1.1*6,10*]docosa-1(21),6,8,10(22),17,19-hexaen-19-yl ester

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-dimethylcarbamoyloxy-benzoic acid (building block A22).

HPLC (Nucleosil C18HD, 20-100% AcCN): retention time=4.36 min

MS: 590 (M+1), 588 (M−1)

¹H-NMR (400 MHz, CDCl₃): 7.32-7.25 (m, 2H), 7.20-7.13 (m, 4H), 7.00 (d, 1H), 6.92 (s, 1H), 6.85-6.78 (m, 3H), 6.04 (d, 1H), 4.24-4.05 (m, 4H), 3.80 (s, 2H), 3.64-3.60 (m, 1H), 3.52-3.47 (m, 2H), 3.20 (dd, 1H), 3.10 (s, 3H), 3.00 (s, 3H), 2.98-2.88 (m, 2H), 2.82-2.70 (m, 2H), 2.10-1.90 (m, 4H), 1.24 (d, 6H).

### Example 27

### 2-{(S)-4-[(R)-1-Hydroxy-2-(3-isopropyl-benzylamino)-ethyl]-2-oxo-11,16-dioxa-3-aza-tricyclo[15.3.1.1*6,10*]docosa-1(21),6,8,10(22),17,19-hexaen-19-yloxy}-N,N-dimethyl-acetamide

The title compound is obtained by an analogous reaction sequence as for example 7, starting from ((1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-dimethylcarbamoylmethoxy-benzoic acid (building block A25).

HPLC (Nucleosil C18HD, 20-100% AcCN): retention time=3.89 min

MS: 604 (M+1), 602 (M−1)

¹H-NMR (400 MHz, CDCl₃): 7.35-7.25 (m, 2H), 7.21-7.16 (m, 4H), 7.04-7.00 (m, 2H), 6.90 (s, 1H), 6.80 (dd, 1H), 6.65 (dd, 1H), 6.62 (s, 1H), 6.07 (d, 1H), 4.70 (s, 2H), 4.25-4.05 (m, 6H), 3.80 (d, 2H), 3.25 (dd, 1H), 3.08 (s, 3H), 3.00 (s, 3H), 2.95-2.82 (m, 2H), 2.82-2.73 (m, 2H), 2.10-1.88 (m, 4H), 1.24 (d, 6H).

### Example 28

### (S)-4-{(R)-1-Hydroxy-2-[(4-isopropyl-pyridin-2-ylmethyl)-amino]-ethyl}-19-oxazol-2-yl-11-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropyl-pyridin-2-ylmethyl)-amino]-2-hydroxy-propyl}-carbamic acid tert-butyl ester (building block C7) and 3-(allyl-benzyloxycarbonyl-amino)-5-oxazol-2-yl-benzoic acid (building block A17).

HPLC (Nucleosil C18HD, 20-100% AcCN): retention time=3.56 min

MS: 570 (M+1), 568 (M−1)

¹H-NMR (400 MHz, CDCl₃): 8.43 (d, 1H), 7.80 (d, 1H), 7.72 (s, 1H), 7.60 (s, 1H), 7.32-7.30 (m, 1H), 7.22 (s, 1H), 7.17 (s, 1H), 7.10-7.20 (m, 2H), 6.92 (s, 1H), 6.80 (d, 1H), 6.73 (s, 1H), 6.08 (d, 1H), 4.40-4.30 (m, 1H), 4.23-3.93 (m, 6H), 3.60-3.52 (m, 1H), 3.38-3.20 (m, 1H), 3.00-2.78 (m, 4H), 2.00-1.70 (m, 4H), 1.23 (d, 6H).

### Example 29

### N—{(S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-N-methyl-methanesulfonamide

### a) (S)-4-{(R)-2-[Benzyloxycarbonyl-(3-isopropyl-benzyl)-amino]-1-hydroxy-ethyl}-19-methanesulfonylamino-2-oxo-11-oxa-3,16-diaza-tricyclo[15.3.1.1′6,10′]docosa-1(21),6,8,10(22),13,17,19-hexaene-16-carboxylic acid benzyl ester

(S)-19-Amino-4-{(R)-2-[benzyloxycarbonyl-(3-isopropyl-benzyl)-amino]-1-hydroxy-ethyl}-2-oxo-11-oxa-3,16-diaza-tricyclo[15.3.1.1′6,10′]docosa-1(21),6,8,10(22),13,17,19-hexaene-16-carboxylic acid benzyl ester (see example 23b) (130 mg, 164 μmol, 1 eq) is dissolved in DCM (0.5 ml) at 0° C. Pyridine (47 μl, 656 μmol, 4.0 eq) and methanesulfonylchloride (57 μl, 721 μmol, 4.4 eq) are added and the reaction is stirred for 1 hour. The reaction mixture is diluted with 1N aq HCl and EtOAc. The organic layer is washed with brine, dried over Na₂SO₄, filtered and concentrated. The residue is purified by column chromatography to give the product.

MS: 863 (M+1), 861 (M−1)

HPLC (Nucleosil C18HD, 20-100% AcCN): retention time=6.29 min

### b) (S)-4-{(R)-2-[Benzyloxycarbonyl-(3-isopropyl-benzyl)-amino]-1-hydroxy-ethyl}-19-(methanesulfonyl-methyl-amino)-2-oxo-11-oxa-3,16-diaza-tricyclo[15.3.1.1′6,10′]docosa-1(21),6,8,10(22),13,17,19-hexaene-16-carboxylic acid benzyl ester

(S)-4-{(R)-2-[Benzyloxycarbonyl-(3-isopropyl-benzyl)-amino]-1-hydroxy-ethyl}-19-methanesulfonylamino-2-oxo-11-oxa-3,16-diaza-tricyclo[15.3.1.1′6,10′]docosa-1(21),6,8,10(22),13,17,19-hexaene-16-carboxylic acid benzyl ester (11 mg, 13 μmol, 1 eq) is dissolved in acetonitrile (0.5 ml). K₂CO₃ (5 mg, 36 μmol, 2.8 eq) and MeI (4 μl, 64 μmol, 5.1 eq) are added. The reaction mixture is stirred then for 5 hours at room temperature. The reaction mixture is concentrated and diluted with water. The crystals formed at 0° C. are collected by filtration to give the product.

MS: 877 (M+1), 875 (M−1)

HPLC (Nucleosil C18HD, 20-100% AcCN): retention time=6.83 min

### c) N-{(S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-N-methyl-methanesulfonamide

The title compound is obtained by an analogous hydrogenation reaction as the last step for example 7, starting from (S)-4-{(R)-2-[benzyloxycarbonyl-(3-isopropyl-benzyl)-amino]-1-hydroxy-ethyl}-19-(methanesulfonyl-methyl-amino)-2-oxo-11-oxa-3,16-diaza-tricyclo[15.3.1.1′6,10′]docosa-1(21),6,8,10(22),13,17,19-hexaene-16-carboxylic acid benzyl ester.

HPLC (Nucleosil C18HD, 20-100% AcCN): retention time=3.91 min

MS: 609 (M+1), 607 (M−1)

### Example 30

### Acetic acid (S)-4-[(R)-1-hydroxy-2-(3-isopropyl-benzylamino)-ethyl]-2-oxo-11,16-dioxa-3-aza-tricyclo[15.3.1.1*6,10*]docosa-1(21),6,8,10(22),17,19-hexaen-19-yl ester

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-acetoxy-5-allyloxy-benzoic acid (building block A18).

HPLC (Nucleosil C18HD, 20-100% AcCN): retention time=4.43 min

MS: 561 (M+1), 559 (M−1)

¹H-NMR (400 MHz, CDCl₃): 7.35-7.14 (m, 6H), 7.00 (d, 1H), 6.90 (s, 1H), 6.83 (s, 1H), 6.81-6.78 (m, 2H), 6.09 (d, 1H), 4.24-4.03 (m, 6H), 3.80 (s, 2H), 3.54-3.50 (m, 1H), 3.23 (dd, 1H), 2.98-2.84 (m, 2H), 2.82-2.72 (m, 2H), 2.32 (s, 3H), 2.10-1.90 (m, 4H), 1.24 (d, 6H).

### Example 31

### (S)-4-[(R)-1-Hydroxy-2-(3-isopropyl-benzylamino)-ethyl]-19-methoxymethyl-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-methoxymethyl-benzoic acid (building block A24).

HPLC (Nucleosil C18HD, 20-100% AcCN): retention time=4.38 min

MS: 547 (M+1), 545 (M−1)

### Example 32

### N-{(S)-4-[(R)-1-Hydroxy-2-(3-isopropyl-benzylamino)-ethyl]-2-oxo-11-oxa-3,16-diaza-tricyclo[15.3.1.1*6,10*]docosa-1(21),6,8,10(22),17,19-hexaen-19-yl}-N-methyl-acetamide

The title compound is obtained by an analogous reaction sequence as for example 7, starting from ((1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-acetyl-methyl-amino-5-(allyl-benzyloxycarbonyl-amino)-benzoic acid (building block A20).

HPLC (Nucleosil C18HD, 20-100% AcCN): retention time=3.57 min

MS: 573 (M+1), 571 (M−1)

### Example 33

### (S)-4-[(R)-1-Hydroxy-2-(3-isopropyl-benzylamino)-ethyl]-19-methoxymethoxy-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-methoxymethoxy-benzoic acid (building block A23)

HPLC (Nucleosil C18HD, 20-100% AcCN): retention time=4.55 min

MS: 563 (M+1), 561 (M−1)

¹H-NMR (400 MHz, CDCl₃): 7.38-7.10 (m, 6H), 7.00 (d, 1H), 6.90 (s, 1H), 6.80 (dd, 1H), 6.77 (s, 1H), 6.62 (s, 1H), 6.09 (d, 1H), 5.22-5.18 (m, 2H), 4.30-4.00 (m, 6H), 4.00-3.80 (m, 2H), 3.50 (s, 3H), 3.37 (dd, 1H), 3.95-3.80 (m, 4H), 2.11-1.90 (m, 4H), 1.25 (d, 6H).

### Example 34

### (S)-4-[(R)-1-Hydroxy-2-(3-isopropyl-benzylamino)-ethyl]-19-(2-oxo-propoxy)-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-(2-oxo-propoxy)-benzoic acid (building block A21).

HPLC (Nucleosil C18HD, 20-100% AcCN): retention time=4.24 min

MS: 575 (M+1), 573 (M−1)

¹H-NMR (400 MHz, d6-DMSO): 7.32-7.12 (m, 6H), 7.03-6.98 (m, 2H), 6.80 (s, 1H), 6.65 (s, 1H), 6.58 (s, 1H), 6.02 (d, 1H), 4.58 (s, 2H), 4.25-4.03 (m, 6H), 3.80 (s, 2H), 3.26 (dd, 1H), 2.95-2.88 (m, 2H), 2.85-2.70 (m, 2H), 2.30 (s, 3H), 2.20-1.80 (m, 4H), 1.28 (d, 6H).

### Example 35

### (S)-19-Chloro-4-[(R)-1-hydroxy-2-(3-isopropyl-benzylamino)-ethyl]-9-methoxy-11-oxa-3,16,18-triaza-tricyclo[15.3.1.1*6,10*]docosa-1(21),6(22),7,9,17,19-hexaen-2-one

### a) 2-(Acetyl-allyl-amino)-N-{(S)-2-(3-allyloxy-4-methoxy-phenyl)-1-[(R)-3-(3-isopropyl-benzyl)-2-oxo-oxazolidin-5-yl]-ethyl}-6-chloro-isonicotinamide

A solution of 800 mg (1.52 mmol) of {(S)-2-(3-allyloxy-4-methoxy-phenyl)-1-[(R)-3-(3-isopropyl-benzyl)-2-oxo-oxazolidin-5-yl]-ethyl}-carbamic acid tert-butyl ester (building block C10) in 6 ml 4N HCl in dioxan is kept 1 h at 50° C. and concentrated in vacuo. The residue is taken up in 10 ml of DCM and treated with 400 mg (1.52 mmol) of 2-(acetyl-allyl-amino)-6-chloro-isonicotinic acid (building block A27), 416 mg (2.72 mmol) HOBt.H₂O, 600 mg (3.05 mmol) EDC.HCl and 1.01 ml (9.2 mmol) N-methylmorpholine and stirred overnight. The mixture is diluted with EtOAc and washed successively with water, 5% aqueous citric acid, water, 5% aqueous NaHCO₃ and water (4×). Evaporation of the mixture and chromatogra on silica gel (EtOAc/hexane 1:2) gives the title compound in the form of a colorless oil.

Rf: (hexane/EtOAc=1/1): 0.27

LC (Nucleosil C-18HD, 4×70 mm, 3 μm, 20-100% AcCN (6 min), 100% AcCN (1.5 min)): 5.880 min

MS (ES) MH⁺=661

### b) (S)-16-Acetyl-19-chloro-4-[(R)-3-(3-isopropyl-benzyl)-2-oxo-oxazolidin-5-yl]-9-methoxy-11-oxa-3,16,18-triaza-tricyclo[15.3.1.1*6,10*]docosa-1(21),6(22),7,9,17,19-hexaen-2-one

To a refluxing solution of 100 mg tricyclohexylphosphine[1,3-bis(2,4,6-trimethylphenyl)-4,5-dihydroimidazol-2-ylidene][benzylidine]ruthenium(IV)dichloride in 200 ml DCM under a nitrogen atmosphere are slowly added 900 mg (1.36 mmol) 2-(acetyl-allyl-amino)-N-{(S)-2-(3-allyloxy-4-methoxy-phenyl)-1-[(R)-3-(3-isopropyl-benzyl)-2-oxo-oxazolidin-5-yl]-ethyl}-6-chloro-isonicotinamide in 10 ml degassed DCM. The mixture is refluxed overnight, cooled to 25° C. and purified via chromatography on silica gel (EtOAc/hexane 1:2) to yield the intermediate olefin as a brown resin. The product is taken up in 10 ml THF and 10 ml EtOH and hydrogenated in the presence of 10 mg 10% Pd—C at 1 atm hydrogen. The mixture is filtered over high-flow and evaporated to yield the product in the form of a beige solid.

Rf: (hexane/EtOAc=1/1(1% AcOH)): 0.50

LC/MS (Nucleosil C-18HD, 4×70 mm, 3 μm, 40-100% AcCN (6 min), 100% AcCN (1.5 min)): 5.19 min;

MS (ES) MH⁺=635, 637

### c) (S)-19-Chloro-4-[(R)-1-hydroxy-2-(3-isopropyl-benzylamino)-ethyl]-9-methoxy-11-oxa-3,16,18-triaza-tricyclo[15.3.1.1*6,10*]docosa-1(21),6(22),7,9,17,19-hexaen-2-one

A stirred mixture of 400 mg (0.63 mmol) (S)-16-acetyl-19-chloro-4-[(R)-3-(3-isopropyl-benzyl)-2-oxo-oxazolidin-5-yl]-9-methoxy-11-oxa-3,16,18-triaza-tricyclo[15.3.1.1*6,10*]docosa-1(21),6(22),7,9,17,19-hexaen-2-one, 754 mg Ba(OH)₂ in 10 ml dioxan and 5 ml water is heated at 80° C. for 18 h. After cooling down the mixture is acidified with 2N H₂SO₄, filtered over high-flow and washed with EtOAc. The organic phase is separated, dried and evaporated to give the crystalline product.

Rf: (DCM/2N NH₃ in MeOH=9/1): 0.20

LC (Nucleosil C-18HD, 4×70 mm, 3 μm, 20-100% AcCN (6 min), 100% AcCN (1.5 min)): 3.981 min

MS (ES) MH+=565, 567

### Example 36

### (S)-19-Chloro-9-hydroxy-4-[(R)-1-hydroxy-2-(3-isopropyl-benzylamino)-ethyl]-11-oxa-3,16,18-triaza-tricyclo[15.3.1.1*6,10*]docosa-1(21),6(22),7,9,17,19-hexaen-2-one

(S)-19-Chloro-4-[(R)-1-hydroxy-2-(3-isopropyl-benzylamino)-ethyl]-9-methoxy-11-oxa-3,16,18-triaza-tricyclo[15.3.1.1*6,10*]docosa-1(21),6(22),7,9,17,19-hexaen-2-one (example 35, 50 mg, 0.088 mmol) is dissolved 5 ml of chloroform and treated with 1 ml BBr₃ (1M in DCM). After 3 h the mixture is quenched with sat. aq. NaHCO₃. The mixture is extracted twice with chloroform. The combined organic layers are dried over Na₂SO₄ and chromatographed on silica gel (CHCl₃/MeOH, 2M NH₃: 9/1) to yield the product in the form of a beige solid.

Rf: (DCM/MeOH(2M NH₃)=9/1): 0.48

LC (Nucleosil C-18HD, 4×70 mm, 3 μm, 20-100% AcCN (6 min), 100% AcCN (1.5 min)): 4.00 min

MS (ES) MH+=553, 555

### Example 37

### (S)-18-Chloro-9-hydroxy-4-[(R)-1-hydroxy-2-(3-isopropyl-benzylamino) ethyl]-3,15,17-triaza-tricyclo[14.3.1.1*6,10*]henicosa-1(20),6(21),7,9,16,18-hexaen-2-one

### a) 2-(Acetyl-allyl-amino)-N-{(S)-2-(3-allyl-4-methoxy-phenyl)-1-[(R)-3-(3-isopropyl-benzyl)-2-oxo-oxazolidin-5-yl]-ethyl}-6-chloro-isonicotinamide

A solution of 200 mg (0.393 mmol) of (S)-5-[(S)-2-(3-allyl-4-methoxy-phenyl)-1-methyl-ethyl]-3-(3-isopropyl-benzyl)-oxazolidin-2-one (building block C11) in 4 ml 4N HCl in dioxan is kept 1 h at 50° C. and concentrated in vacuo. The residue is taken up in 5 ml DCM and treated with 100 mg (0.393 mmol) 2-(acetyl-allyl-amino)-6-chloro-isonicotinic acid (building block A27), 107 mg (0.7 mmol) HOBt.H₂O, 154 mg (0.80 mmol) EDC.HCl and 0.22 ml (2 mmol) N-methylmorpholine and stirred overnight. The mixture is diluted with EtOAc and washed successively with water, 5% aqueous citric acid, water, 5% aqueous NaHCO₃ and water (4×). Evaporation of the mixture and chromatography of the residue on silica gel (EtOAc/hexane 1:2) gives the title compound in the form of a colorless oil.

Rf: (hexane/EtOAc=1/1): 0.23

LC (Nucleosil C-18HD, 4×70 mm, 3 μm, 20-100% AcCN (6 min), 100% AcCN (1.5 min)): 6.345 min

MS (ES) [MH]⁺ 645

### b) (S)-15-Acetyl-18-chloro-4-[(R)-3-(3-isopropyl-benzyl)-2-oxo-oxazolidin-5-yl]-9-methoxy-3,15,17-triaza-tricyclo[14.3.1.1*6,10*]henicosa-1(20),6(21),7,9,16,18-hexaen-2-one

To a refluxing solution of 50 mg (0.058 mmol) tricyclohexylphosphine[1,3-bis(2,4,6-trimethylphenyl)-4,5-dihydroimidazol-2-ylidene][benzylidine]ruthenium(IV)dichloride in 100 ml DCM under a nitrogen atmosphere are slowly added 245 mg (0.38 mmol) 2-(acetyl-allyl-amino)-N-{(S)-2-(3-allyl-4-methoxy-phenyl)-1-[(R)-3-(3-isopropyl-benzyl)-2-oxo-oxazolidin-5-yl]-ethyl}-6-chloro-isonicotinamide in 10 ml degassed DCM. The mixture is refluxed 6 h, cooled to 25° C. and purified via chromatography on silica gel (EtOAc/hexane 1:2) to yield 182 mg the intermediate olefin as a brown resin. The product is taken up in 10 ml THF and 10 ml EtOH and hydrogenated in the presence of 10 mg 10% Pd—C at 1 atm hydrogen. The mixture is filtered over high-flow, and the filtrate is evaporated to yield the title compound in the form of a beige solid.

Rf: (hexane/EtOAc=1/1): 0.32

LC (Nucleosil C-18HD, 4×70 mm, 3 μm, 20-100% AcCN (6 min), 100% AcCN (1.5 min)): 5.939 min

MS (ES) [MH]⁺ 619

### c) (S)-18-Chloro-9-hydroxy-4-[(R)-1-hydroxy-2-(3-isopropyl-benzylamino)-ethyl]-3,15,17-triaza-tricyclo[14.3.1.1*6,10*]henicosa-1(20),6(21),7,9,16,18-hexaen-2-one

A stirred mixture of 90 mg (0.168 mmol) (S)-15-Acetyl-18-chloro-9-hydroxy-4-[(R)-3-(3-isopropyl-benzyl)-2-oxo-oxazolidin-5-yl]-3,15,17-triaza-tricyclo[14.3.1.1*6,10*]henicosa-1(20),6(21),7,9,16,18-hexaen-2-one, 390 mg (1.17 mmol) Ba(OH)₂ in 4 ml dioxan and 2 ml water is heated at 100° C. for 4 h. The mixture is cooled down and acidified with 2N H₂SO₄, filtered over high-flow and washed with EtOAc. The organic phase is separated, dried and crystallised from MeOH to give the title compound.

Rf (DCM/MeOH/Et₃N=90:9:1) 0.08

LC (Nucleosil C-18HD, 4×70 mm, 3 μm, 20-100% AcCN (6 min), 100% AcCN (1.5 min)): 3.842 min

MS (ES) [MH]⁺ 603

### Example 38

### (S)-4-((R)-1-Hydroxy-2-[(4-isopropyl-pyridin-2-ylmethyl)-amino]-ethyl}-18-(2-oxo-pyrrolidin-1-yl)-15-oxa-3-aza-tricyclo[14.3.1.1*6,10*]henicosa-1(19),6,8,10(21),16(20),17-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 3-allyloxy-5-(2-oxo-pyrrolidin-1-yl)-benzoic acid (building block A13).

MS (LC/MS): 571=[M+H]⁺

¹H-NMR (400 MHz, d6-DMSO): 8.35 (d, 1H) 8.02 (d, 1H), 7.38 (s, 1H), 7.31-7.30 (m, 2H), 7.20 (s, 1H), 7.11 (t, 1H), 7.03 (d, 1H), 6.96 (d, 1H), 6.51 (s, 1H), 5.06 (br s, 1H), 4.34-4.27 (m, 1H), 4.13-4.03 (m, 2H), 3.85 (s, 2H), 3.72 (t, 2H), 3.70-3.64 (m, 1H), 3.11 (br d, 1H), 3.92-3.82 (m, 1H), 2.75-2.55 (m, 5H), 2.44 (t, 2H), 2.03-1.96 (m, 2H), 1.95-1.78 (m, 2H), 1.53-1.33 (m, 2H), 1.19 (d, 6H).

### Example 39

### (S)-4-[(R)-1-Hydroxy-2-(3-isopropyl-benzylamino)-ethyl]-2-oxo-15-oxa-3-aza-tricyclo[14.3.1.1*6,10*]henicosa-1(20),6,8,10(21),16,18-hexaene-18-carboxylic acid methyl ester

The title compound can be obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 5-allyloxy-isophthalic acid monomethyl ester (prepared as described in steps a-b for building block A28).

MS (LC/MS): 545=[M+H]⁺

¹H-NMR (400 MHz, CDCl₃): 8.02 (2, 1H), 7.65 (s, 1H), 7.35-7.14 (m, 8H), 6.84 (s, 1H), 5.89 (d, 1H), 4.36-4.28 (m, 1H), 4.28-4.22 (m, 2H), 3.94 (s, 3H), 3.87 (s, 2H), 3.70-3.60 (m, 1H), 3.17-3.07 (m, 2H), 2.95-2.75 (m, 5H), 2.08-1.92 (m, 2H), 1.71-1.63 (m, 2H), 1.29 (d, 6H).

### Example 40

### (S)-4-{(R)-1-Hydroxy-2-[(4-isopropyl-pyridin-2-ylmethyl)-amino]-ethyl}-18-methyl-3,15,17-triaza-tricyclo[14.3.1.1*6,10*]henicosa-1(19),6,8,10(21),16(20),17-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 2-allylamino-6-methyl-isonicotinic acid hydrochloride (building block A2).

MS (LC/MS): 502=[M+H]⁺

¹H-NMR (400 MHz, CDCl₃): 8.48 (d, 1H), 7.38 (s, 1H), 7.33-7.26 (m, 2H), 7.19-7.14 (m, 3H), 7.09 (d, 1H), 6.73 (s, 1H), 6.00 (d, 1H), 5.92 (s, 1H), 5.09 (t, 1H), 4.40-4.33 (m, 1H), 4.08 (d, 1H), 4.00 (d, 1H), 3.71-3.66 (m, 1H), 3.32-3.24 (m, 2H), 3.19 (dd, 1H), 3.10 (dd, 1H), 2.95-2.75 (m, 5H), 2.35 (s, 3H), 2.02-1.85 (m, 2H), 1.58-1.49 (m, 2H), 1.29 (d, 6H).

### Example 41

### (S)-4-[(R)-1-Hydroxy-2-(3-isopropyl-benzylamino)-ethyl]-18-oxazol-2-yl-15-oxa-3-aza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-allyloxy-5-oxazol-2-yl-benzoic acid (building block A28).

MS (LC/MS): 554=[M+H]⁺

¹H-NMR (400 MHz, d6-DMSO): 8.19 (s, 1H), 8.18 (d, 1H), 7.64 (s, 1H), 7.40-7.39 (m, 2H), 7.36 (s, 1H), 7.21-7.18 (m, 2H), 7.16-7.12 (m, 2H), 7.07 (s, 1H), 7.05 (s, 1H), 6.98 (d, 1H), 6.86 (s, 1H), 4.42-4.35 (m, 1H), 4.20-4.10 (m, 2H), 3.74 (s, 2H), 3.69-3.64 (m, 1H), 3.13 (dd, 1H), 2.88-2.78 (m, 1H), 2.75-2.60 (m, 5H), 1.99-1.80 (m, 2H), 1.55-1.38 (m, 2H), 1.17 (d, 6H).

### Example 42

### (S)-4-{(R)-1-Hydroxy-2-[(4-isopropyl-pyridin-2-ylmethyl)-amino]-ethyl}-18-oxazol-2-yl-15-oxa-3-aza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 3-allyloxy-5-oxazol-2-yl-benzoic acid (building block A28).

MS (LC/MS): 555=[M+H]⁺

¹H-NMR (400 MHz, d6-DMSO): 8.37 (d, 1H), 8.21 (s, 2H), 7.65 (s, 1H), 7.42-7.32 (m, 4H), 7.17-7.06 (m, 3H), 7.00 (d, 1H), 6.87 (s, 1H), 5.05 (br s, 1H), 4.41-4.35 (m, 1H), 4.20-4.10 (m, 2H), 3.83 (s, 2H), 3.67 (d, 1H), 3.14 (d, 1H), 2.86 (br s, 1H), 2.75-2.60 (m, 5H), 2.00-1.80 (m, 2H), 1.55-1.38 (m, 2H), 1.18 (d, 6H).

### Example 43

### (S)-4-[(R)-1-Hydroxy-2-(3-isopropyl-benzylamino)-ethyl]-2-oxo-11,16-dioxa-3-aza-tricyclo[15.3.1.1*6,10*]docosa-1(21),6,8,10(22),17,19-hexaene-19-carboxylic acid dimethylamide

A solution of 42 mg (0.1 mmol) (S)-4-(S)-oxiranyl-2-oxo-11,16-dioxa-3-aza-tricyclo[15.3.1.1*6,10*]docosa-1(21),6,8,10(22),17,19-hexaene-19-carboxylic acid dimethylamide (building block E1) in 74 mg (0.5 mmol, 5 eq.) 3-isopropyl-benzylamine is heated at 80° C. for 2 h. The reaction mixture is diluted with 1.5 ml DCM and purified by preparative thin layer chromatography on silica gel (DCM/MeOH 90/10) to give the product in the form of a colorless solid.

Rf: (DCM/MeOH=90/10): 0.37

MS (ES+): 574=[M+H]⁺

¹H-NMR (400 MHz, d6-DMSO): 8.11 (d, 1H), 7.20-7.13 (m, 3H), 7.12-7.06 (m, 1H), 7.07-7.03 (m, 2H), 7.01-6.97 (m, 2H), 6.92 (t, 1H), 6.81 (d, 1H), 6.74 (dd, 1H), 5.00 (br s, 1H), 4.43-4.34 (m, 1H), 4.26-4.19 (m, 1H), 4.17-4.08 (m 1H), 4.01-3.88 (m, 2H), 3.70 (s, 2H), 3.64-3.58 (m, 1H), 3.02-2.92 (m, 4H), 2.88-2.79 (m, 4H), 2.72-2.62 (m, 2H), 2.58-2.63 (m, 1H), 1.88-1.64 (m, 4H), 1.17 (d, 6H).

### Example 44

### (S)-4-[(R)-2-(3-Cyclopropyl-benzylamino)-1-hydroxy-ethyl]-2-oxo-11,16-dioxa-3-aza-tricyclo[15.3.1.1*6,10*]docosa-1(21),6,8,10(22),17,19-hexaene-19-carboxylic acid dimethylamide

The title compound is obtained by an analogous reaction sequence as for example 43, starting from (S)-4-(S)-oxiranyl-2-oxo-11,16-dioxa-3-aza-tricyclo[15.3.1.1*6,10*]docosa-1(21),6,8,10(22),17,19-hexaene-19-carboxylic acid dimethylamide (building block E1) and 3-cyclopropyl-benzylamine (building block D2).

Colorless solid.

Rf: (DCM/MeOH=90/10): 0.35

MS (ES+): 572=[M+H]⁺

¹H-NMR (400 MHz, d6-DMSO): 8.11 (d, 1H), 7.19-7.09 (m, 2H), 7.07-7.02 (m, 2H), 7.01-6.97 (m, 3H), 6.92 (t, 1H), 6.88 (d, 1H), 6.81 (d, 1H), 6.74 (dd, 1H), 4.97 (br s, 1H), 4.43-4.34 (m, 1H), 4.26-4.19 (m, 1H), 4.17-4.08 (m 1H), 4.01-3.88 (m, 2H), 3.66 (s, 2H), 3.63-3.56 (m, 1H), 3.02-2.91 (m, 4H), 2.88-2.71 (m, 4H), 2.72-2.59 (m, 2H), 1.91-1.63 (m, 5H), 0.93-0.87 (m, 2H), 0.66-0.61 (m, 2H).

### Example 45

### (S)-4-{(R)-2-[(6-Ethyl-pyrimidin-4-ylmethyl)-amino]-1-hydroxy-ethyl}-2-oxo-11,16-dioxa-3-aza-tricyclo[15.3.1.1*6,10*]docosa-1(21),6,8,10(22),17,19-hexaene-19-carboxylic acid dimethylamide

The title compound is obtained by an analogous reaction sequence as for example 43, starting from (S)-4-(S)-oxiranyl-2-oxo-11,16-dioxa-3-aza-tricyclo[15.3.1.1*6,10*]docosa-1(21),6,8,10(22),17,19-hexaene-19-carboxylic acid dimethylamide (building block E1) and C-(6-ethyl-pyrimidin-4-yl)-methylamine (building block D3). Orange solid.

Rf: (DCM/MeOH=90/10): 0.28

MS (ES+): 562=[M+H]⁺

¹H-NMR (400 MHz, d6-DMSO): 8.91 (d, 1H), 8.11 (d, 1H), 7.42 (s, 1H), 7.17 (t, 1H), 7.06-7.03 (m, 1H), 7.01-6.99 (m, 1H), 6.98-6.96 (m, 1H), 6.94-6.91 (m, 1H), 6.82 (d, 1H), 6.75 (dd, 1H), 5.09-5.01 (m, 1H), 4.42-4.33 (m, 1H), 4.25-4.19 (m, 1H), 4.17-4.09 (m, 1H), 4.02-3.90 (m, 2H), 3.79 (d, 2H), 3.66-3.56 (m, 1H), 3.01 (dd, 1H), 2.95 (s, 3H), 2.84 (s, 3H), 2.76-2.64 (m, 4H), 2.60-2.53 (m, 1H), 1.88-1.64 (m, 4H), 1.19 (t, 3H).

### Example 46

### (S)-4-[(R)-1-Hydroxy-2-(3-isopropyl-benzylamino)-ethyl]-18-oxazol-2-yl-3,15-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-(allyl-benzyloxycarbonyl-amino)-5-oxazol-2-yl-benzoic acid (building block A17).

MS (LC/MS): 553=[M+H]⁺

¹H-NMR (400 MHz, d6-DMSO): 8.17 (s, 1H), 8.06 (d, 1H), 7.40 (s, 1H), 7.34 (s, 1H), 7.25-7.16 (m, 6H), 7.09-7.06 (m, 2H), 7.02 (d, 1H), 6.43 (d, 1H), 6.33 (t, 1H), 4.96-4.90 (m, 1H), 4.14-4.06 (m, 1H), 3.74 (s, 2H), 3.66-3.60 (m, 1H), 3.14 (dd, 1H), 3.08-3.02 (m, 1H), 2.90-2.79 (m, 1H), 2.74-2.61 (m, 6H), 1.98-1.88 (m, 1H), 1.83-1.73 (m, 1H), 1.52-1.40 (m, 1H), 1.20-1.10 (m, 1H), 1.17 (d, 6H).

### Example 47

### (S)-19-Ethoxymethyl-4-[(R)-1-hydroxy-2-(3-isopropyl-benzylamino)-ethyl]-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-ethoxymethyl-benzoic acid (building block A30).

HPLC (Nucleosil C18HD, 20-100% ACN): retention time=4.69 min

MS (ES+): 561=[M+H]⁺

¹H-NMR (400 MHz, d6-DMSO): 8.01 (d, 1H), 7.20-6.98 (m, 7H), 6.93 (s, 1H), 6.87 (s, 1H), 6.80 (d, 1H), 6.73 (d, 1H), 4.98 (s, 1H), 4.40 (s, 2H), 4.40-4.30 (m, 1H), 4.28-4.26 (m, 1H), 4.13-4.02 (m, 1H), 4.00-3.85 (m, 2H), 3.70 (s, 2H), 3.65-3.55 (m, 1H), 3.45 (q, 2H), 3.03-2.97 (m, 1H), 2.88-2.79 (m, 1H), 2.75-2.62 (m, 2H), 2.60-2.50 (m, 1H), 1.85-1.65 (m, 4H), 1.17 (d, 6H), 1.13 (t, 3H).

### Example 48

### (S)-4-[(R)-1-Hydroxy-2-(3-isopropyl-benzylamino)-ethyl]-19-(2,2,2-trifluoro-ethoxymethyl)-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-(2,2,2-trifluoro-ethoxymethyl)-benzoic acid (building block A31).

HPLC (Nucleosil C18HD, 20-100% ACN): retention time=4.86 min

MS (ES+): 615=[M+H]⁺

¹H-NMR (400 MHz, d6-DMSO): 8.02 (d, 1H), 7.20-7.00 (m, 8H), 6.99-6.96 (m, 2H), 6.88 (s, 1H), 6.80 (d, 1H), 6.73 (d, 1H), 4.94 (s, 1H), 4.58 (s, 2H), 4.40-4.32 (m, 1H), 4.25-4.17 (m, 1H), 4.13-4.02 (m, 3H), 4.00-3.85 (m, 2H), 3.67 (s, 2H), 3.64-3.55 (m, 1H), 3.00-2.95 (m, 1H), 2.88-2.76 (m, 1H), 2.75-2.60 (m, 2H), 2.58-2.52 (m, 1H), 1.85-1.60 (m, 4H), 1.16 (d, 6H).

### Example 49

### (S)-4-[(R)-1-Hydroxy-2-(3-isopropyl-benzylamino)-ethyl]-19-methoxymethoxymethyl)-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-methoxymethoxymethyl-benzoic acid (building block A32).

HPLC (Nucleosil C18HD, 20-100% ACN): retention time=4.42 min

MS (ES+): 579=[M+H]⁺

¹H-NMR (400 MHz, d6-DMSO): 8.01 (d, 1H), 7.20-6.98 (m, 7H), 6.95 (s, 1H), 6.90 (s, 1H), 6.80 (d, 1H), 6.73 (d, 1H), 4.95 (s, 1H), 4.63 (s, 2H), 4.45 (s, 2H), 4.40-4.32 (m, 1H), 4.25-4.18 (m, 1H), 4.13-4.04 (m, 1H), 3.99-3.78 (m, 2H), 3.70 (s, 2H), 3.64-3.58 (m, 1H), 3.28 (s, 3H), 3.02-2.90 (m, 1H), 2.87-2.79 (m, 1H), 2.72-2.62 (m, 2H), 2.60-2.50 (m, 1H), 1.87-1.65 (m, 4H), 1.18 (d, 6H).

### Example 50

### (S)-4-[(R)-1-Hydroxy-2-(3-isopropyl-benzylamino)-ethyl]-19-methoxymethyl)-11-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-methoxymethyl-benzoic acid (building block A33).

HPLC (Nucleosil C18HD, 20-100% ACN): retention time=3.74 min

MS (ES+): 546=[M+H]⁺

¹H-NMR (400 MHz, d6-DMSO): 7.90 (d, 1H), 7.20-7.00 (m, 8H), 6.78 (d, 1H), 6.72 (d, 1H), 6.60-6.52 (m, 2H), 6.47 (s, 1H), 5.82 (t, 1H), 4.88 (d, 1H), 4.28-4.18 (m, 3H), 3.95-3.70 (m, 2H), 3.66 (s, 2H), 3.60-3.52 (m, 1H), 3.45-3.35 (m, 1H), 3.23 (s, 3H), 3.05-2.90 (m, 2H), 2.88-2.75 (s, 1H), 2.70-2.55 (m, 2H), 2.52-2.48 (m, 1H), 1.75-1.42 (m, 4H), 1.17 (d, 6H).

### Example 51

### (S)-4-[(R)-1-Hydroxy-2-(3-isopropyl-benzylamino)-ethyl]-19-oxazol-2-yl-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-oxazol-2-yl-benzoic acid (building block A28).

HPLC (Nucleosil C18HD, 20-100% ACN): retention time=4.32 min

MS (ES+): 570=[M+H]⁺

¹H-NMR (400 MHz, d6-DMSO): 8.40-8.25 (m, 2H), 7.65 (s, 1H), 7.45 (s, 1H), 7.37 (s, 1H), 7.20-7.06 (m, 5H), 7.05-6.95 (m, 2H), 6.81 (d, 1H), 6.74 (d, 1H), 5.00 (s, 1H), 4.45-4.35 (m, 1H), 4.25-4.10 (m, 2H), 4.00-3.90 (m, 2H), 3.70 (s, 2H), 3.65-3.55 (s, 1H), 3.05-2.95 (m, 1H), 2.75-2.65 (m, 1H), 2.75-2.60 (m, 2H), 2.60-2.52 (m, 1H), 1.90-1.65 (m, 4H), 1.13 (d, 6H).

### Example 52

### (S)-4-[(R)-1-Hydroxy-2-(3-isopropyl-benzylamino)-ethyl]-19-oxazol-5-yl-11-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-oxazol-5-yl-benzoic acid (building block A35).

HPLC (Nucleosil C18HD, 20-100% ACN): retention time=3.82 min

MS (ES+): 569=[M+H]⁺

¹H-NMR (400 MHz, d6-DMSO): 8.37 (s, 1H), 8.02 (d, 1H), 7.50 (s, 1H), 7.25-6.90 (m, 8H), 6.77 (d, 1H), 6.72 (d, 1H), 6.58 (s, 1H), 6.08 (m, 1H), 4.92 (s, 1H), 4.30-4.15 (m, 1H), 4.00-3.85 (m, 2H), 3.68 (s, 2H), 3.65-3.55 (m, 1H), 3.50-3.35 (m, 1H), 3.10-2.90 (m, 2H), 2.90-2.75 (m, 1H), 2.75-2.60 (m, 2H), 2.58-2.50 (m, 1H), 1.80-1.45 (m, 4H), 1.13 (d, 6H).

### Example 53

### (S)-4-[(R)-1-Hydroxy-2-(3-isopropyl-benzylamino)-ethyl]-19-oxazol-5-yl-11,16-dioxa-3-aza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyl-5-oxazol-5-yl-benzoic acid (building block A34).

HPLC (Nucleosil C18HD, 20-100% ACN): retention time=4.17 min

MS (ES+): 570=[M+H]⁺

¹H-NMR (400 MHz, d6-DMSO): 8.46 (s, 1H), 8.30 (d, 1H), 7.72 (s, 1H), 7.20-7.00 (m, 9H), 6.80 (d, 1H), 6.72 (d, 1H), 4.45-4.35 (m, 1H), 4.30-4.20 (m, 1H), 4.20-4.10 (m, 1H), 4.07-3.76 (m, 5H), 3.12-2.98 (m, 1H), 2.96-2.90 (m, 1H), 2.88-2.65 (m, 3H), 1.90-1.65 (m, 4H), 1.18 (d, 6H).

### Example 54

### (S)-18-Chloro-4-[(R)-1-hydroxy-2-(3-isopropyl-benzylamino)-ethyl]-3,15,17-triaza-tricyclo[14.3.1.1*6,10*]henicosa-1(20),6,8,10(21),16,18-hexaen-2-one

The title compound is obtained by an analogous reaction sequence as for example 35, starting from {(S)-2-(3-allyl-phenyl)-1-[(R)-3-(3-isopropyl-benzyl)-2-oxo-oxazolidin-5-yl]-ethyl}-carbamic acid tert-butyl ester (building block C12) and 2-(acetyl-allyl-amino)-6-chloro-isonicotinic acid (building block A27).

Rf: (DCM/MeOH(2M NH3)=9/1): 0.25

LC (Nucleosil C-18HD, 4×70 mm, 3 μm, 40-100% MeCN (6 min), 100% MeCN (1.5 min)): 3.087 min

MS (ES+): 521/523=[M+H]⁺

### Example 55

### (S)-19-Chloro-4-[(R)-1-hydroxy-2-(3-isopropyl-benzylamino)-ethyl]-8-methyl-11-oxa-3,16,18-triaza-tricyclo[15.3.1.1*6,10*]docosa-1(21),6,8,10(22),17,19-hexaen-2-one

The title compound is obtained by an analogous reaction sequence as for example 35, starting from [(1S,2R)-1-(3-allyloxy-5-methyl-benzyl)-2-hydroxy-3-(3-isopropyl-benzylamino)-propyl]-carbamic acid tert-butyl ester (building block C13) and 2-(acetyl-allyl-amino)-6-chloro-isonicotinic acid (building block A27).

Rf: (EtOAc/MeOH=19/1): 0.22

LC (Nucleosil C-18HD, 4×70 mm, 3 μm, 20-100% MeCN (6 min), 100% MeCN (1.5 min)): 4.512 min

MS (ES+): 551/553=[M+H]⁺

### Example 56

### (S)-4-[(R)-1-Hydroxy-2-(3-isopropyl-benzylamino)-ethyl]-19-(2-oxo-propoxy)-11-oxa-3,16-diaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-(2-oxo-propoxy)-benzoic acid (building block A36).

HPLC (Nucleosil C18HD, 4×70 mm, 3 μm, 20-100% AcCN (6 min), 100% AcCN (1.5 min)) retention time=3.79 min

MS (ES+): 574=[M+H]⁺

¹H-NMR (400 MHz, CDCl₃): 7.38-7.07 (m, 5H), 7.02 (d, 1H), 6.92 (s, 1H), 6.82 (dd, 1H), 6.77 (s, 1H), 6.33 (s, 1H), 6.23 (dd, 1H), 6.02 (d, 1H), 4.58 (s, 2H), 4.28-4.17 (m, 2H), 4.17-4.09 (m, 1H), 3.96-3.89 (m, 1H), 3.81 (s, 2H), 3.51-3.45 (m, 1H), 3.33-3.22 (m, 3H), 2.98-2.85 (m, 2H), 2.82-2.70 (m, 2H), 2.28 (s, 3H), 1.99-1.80 (m, 2H), 1.79-1.65 (m, 1H), 1.27 (d, 6H).

### Example 57

### (S)-4-[(R)-1-Hydroxy-2-(3-isopropyl-benzylamino)-ethyl]-19-(3-oxo-butyl)-11,16-dioxa-3-aza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-(3-oxo-butyl)-benzoic acid (building block A38).

MS (ES+): 573=[M+H]⁺

¹H-NMR (400 MHz, d6-DMSO): 7.95 (d, 1H), 7.20-7.02 (m, 5H), 6.98 (s, 1H), 6.90-6.68 (m, 5H), 4.85 (bs, 1H), 4.38-4.25 (m, 1H), 4.25-4.17 (m, 1H), 4.10-3.98 (m, 1H), 3.98-3.84 (m, 2H), 3.69 (s, 2H), 3.62-3.55 (m, 1H), 3.00-2.93 (m, 1H), 2.90-2.77 (m, 1H), 2.75-2.60 (m, 6H), 2.58-2.52 (m, 1H), 2.06 (s, 3H), 1.85-1.62 (m, 4H), 1.16 (d, 6H).

### Example 58

### (S)-4-[(R)-1-Hydroxy-2-(3-isopropyl-benzylamino)-ethyl]-19-methyl-11,16-dioxa-3,18-diaza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 2-allyloxy-6-methyl-isonicotinic acid (building block A37).

HPLC (Nucleosil C18HD, 4×70 mm, 3 μm, 20-100% AcCN (6 min), 100% AcCN (1.5 min)) retention time=3.62 min

MS (ES+): 518=[M+H]⁺

¹H-NMR (400 MHz, d6-DMSO): 8.11 (d, 1H), 7.21-7.04 (d, 5H), 6.85-6.67 (m, 4H), 6.51 (s, 1H), 4.98 (s, 1H), 4.50-4.36 (m, 1H), 4.27-4.12 (m, 2H), 4.01-3.85 (m, 2H), 3.69 (s, 2H), 3.64-3.55 (m, 1H), 3.01 (dd, 1H), 2.90-2.78 (m, 1H), 2.70-2.50 (m, 3H), 2.34 (s, 3H), 1.85-1.70 (m, 3H), 1.70-1.59 (m, 1H), 1.16 (d, 6H).

### Example 59

### (S)-19-Acetyl-4-[(R)-1-hydroxy-2-(3-isopropyl-benzylamino)-ethyl]-11,16-dioxa-3-aza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-acetyl-5-allyloxy-benzoic acid (building block A39).

MS (ES+): 545=[M+H]⁺

¹H-NMR (400 MHz, d6-DMSO): 8.40 (d, 1H), 7.62 (s, 1H), 7.47 (s, 1H), 7.27 (s, 1H), 7.23-7.05 (m, 5H), 7.00 (s, 1H), 6.81 (d, 1H), 6.74 (d, 1H), 5.08 (bs, 1H), 4.45-4.37 (m, 1H), 4.25-4.17 (m, 2H), 4.00-3.92 (m, 2H), 3.74 (s, 2H), 3.68-3.60 (m, 1H), 3.02-2.95 (m, 1H), 2.88-2.76 (m, 1H), 2.75-2.64 (m, 2H), 2.65-2.54 (m, 1H), 2.54 (s, 3H), 1.86-1.60 (m, 4H), 1.16 (d, 6H).

### Example 60

### (S)-4-[(R)-1-Hydroxy-2-(3-isopropyl-benzylamino)-ethyl]-19-(2-oxo-propyl)-11,16-dioxa-3-aza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-allyloxy-5-(2-oxo-propyl)-benzoic acid (building block A40).

HPLC (Nucleosil C18HD, 4×70 mm, 3 μm, 20-100% AcCN (6 min), 100% AcCN (1.5 min)) retention time=4.19 min

MS (ES+): 559=[M+H]⁺

### Example 61

### (S)-4-[(R)-1-Hydroxy-2-(3-isopropyl-benzylamino)-ethyl]-19-(2-oxo-butyl)-11-oxa-3,16-diaza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 3-(allyl-benzyloxycarbonyl-amino)-5-(3-oxo-butyl)-benzoic acid (building block A41).

HPLC (Nucleosil C18HD, 4×70 mm, 3 μm, 20-100% AcCN (6 min), 100% AcCN (1.5 min)) retention time=3.58 min

MS (ES+): 572=[M+H]⁺

¹H-NMR (400 MHz, d6-DMSO): 7.40-6.95 (m, 6H), 6.72-6.68 (m, 3H), 6.54-6.40 (m, 2H), 5.11 (s, 1H), 4.62-4.48 (m, 2H), 4.25-4.15 (m, 1H), 4.05-3.95 (m, 2H), 3.82-3.73 (m, 1H), 3.75 (s, 2H), 3.70-3.63 (m, 2H), 3.40-3.25 (m, 1H), 3.20-2.55 (m, 10H), 2.07 (s, 3H), 1.80-1.55 (m, 4H), 1.22 (d, 6H).

### Example 62

### (S)-4-[(R)-1-Hydroxy-2-(3-isopropyl-benzylamino)-ethyl]-19-methoxy-methyl-11-oxa-3,16,18-triaza-tricyclo[15.3.1.1*6.10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(3-isopropylbenzyl)-amino]-2-hydroxy-propyl}-carbamic acid tert-butyl ester (building block C6) and 2-allylamino-6-methoxymethyl-isonicotinic acid (building block A42).

MS (ES+): 547=[M+H]⁺

HPLC (Nucleosil C18HD, 4×70 mm, 3 μm, 20-100% AcCN (6 min), 100% AcCN (1.5 min)) retention time=3.08 min

¹H-NMR (400 MHz, d6-DMSO): 8.17 (d, 1H), 7.21-7.02 (m, 6H), 6.84-6.70 (m, 2H), 6.65 (t, 1H), 6.54 (s, 1H), 6.27 (s, 1H), 4.96 (s, 1H), 4.30-4.20 (m, 1H), 4.23 (s, 2H), 4.00-3.85 (m, 2H), 3.68 (s, 2H), 3.63-3.54 (m, 1H), 3.50-3.31 (m, 2H), 3.32 (s, 3H), 3.10-2.95 (m, 2H), 2.91-2.81 (m, 1H), 2.73-2.59 (m, 2H), 1.86-1.60 (m, 3H), 1.60-1.45 (m, 1H), 1.18 (d, 6H).

### Example 63

### (S)-4-[(R)-1-Hydroxy-2-(3-isopropyl-benzylamino)-ethyl]-18-methoxymethyl-3,15-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(3-isopropyl-benzyl)-amino]-2-hydroxy-propyl}-carbamic acid tert-butyl ester (building block C5) and 3-(allyl-benzyloxycarbonyl-amino)-5-methoxymethyl-benzoic acid (building block A33).

MS (ES+): 530=[M+H]⁺

¹H-NMR (400 MHz, d6-DMSO): 7.90 (d, 1H), 7.40 (s, 1H), 7.24-7.22 (m, 2H), 7.19-7.15 (m, 2H), 7.11 (d, 1H), 7.06 (d, 1H), 7.01 (d, 1H), 6.59 (s, 1H), 6.53 (s, 1H), 6.24 (s, 1H), 5.95 (t, 1H), 4.95 (br s, 1H), 4.23 (s, 2H), 4.15-4.03 (m, 1H), 3.75 (s, 2H), 3.63 (br s, 1H), 3.23 (s, 3H), 3.20 (dd, 1H), 3.13 (dd, 1H), 3.05-2.92 (m, 1H), 2.93-2.82 (m, 1H), 2.74-2.61 (m, 5H), 1.97-1.87 (m, 1H), 1.81-1.72 (m, 1H), 1.52-1.43 (m, 1H), 1.21 (d, 6H), 1.15-1.04 (m, 1H).

### Example 64

### (S)-4-{(R)-1-Hydroxy-2-[(4-isopropyl-pyridin-2-ylmethyl)-amino]-ethyl}-18-methoxymethyl-3,15-diaza-tricyclo[14.3.1.1*6,10*]henicosa-1(19),6,8,10(21),16(20),17-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 3-(allyl-benzyloxycarbonyl-amino)-5-methoxymethyl-benzoic acid (building block A33).

MS (ES+): 531=[M+H]⁺

¹H-NMR (400 MHz, CDCl₃): 8.48 (d, 1H), 7.32-7.27 (m, 2H), 7.21 (s, 1H), 7.18 (s, 1H), 7.14 (d, 1H), 7.08 (d, 1H), 7.04 (s, 1H), 6.65 (s, 1H), 6.34 (s, 1H), 5.95 (d, 1H), 4.42-4.34 (m, 1H), 4.36 (s, 2H), 4.12 (br s, 1H), 4.08 (d, 1H), 3.99 (d, 1H), 3.66-3.62 (m, 1H), 3.36 (s, 3H), 3.35-3.17 (m, 3H), 3.09 (dd, 1H), 2.98-2.72 (m, 5H), 2.01-1.84 (m, 2H), 1.59-1.48 (m, 2H), 1.29 (d, 6H).

### Example 65

### (S)-4-{(R)-1-Hydroxy-2-[(4-isopropyl-pyridin-2-ylmethyl)-amino]-ethyl}-18-oxazol-2-yl-3,15-diaza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 3-(allyl-benzyloxycarbonyl-amino)-5-oxazol-2-yl-benzoic acid (building block A17).

MS (ES+): 554=[M+H]⁺

¹H-NMR (400 MHz, d6-DMSO): 8.46 (d, 1H), 8.20-8.18 (m, 2H), 7.43 (s, 1H), 7.40 (s, 1H), 7.35 (s, 1H), 7.25-7.18 (m, 4H), 7.08 (d, 1H), 7.03 (d, 1H), 6.42 (s, 1H), 6.37 (t, 1H), 5.50 (br s, 1H), 4.13 (s, 2H), 4.12-4.05 (m, 1H), 3.76 (br t, 1H), 3.43-3.34 (m, 1H), 3.20 (dd, 1H), 3.09-2.85 (m, 4H), 2.75-2.67 (m, 3H), 1.99-1.89 (m, 1H), 1.84-1.75 (m, 1H), 1.52-1.41 (m, 1H), 1.21 (d, 6H), 1.20-1.09 (m, 1H).

### Example 66

### (S)-9-Fluoro-4-[(R)-1-hydroxy-2-(3-isopropyl-benzylamino)-ethyl]-18-methyl-3,15,17-triaza-tricyclo[14.3.1.1*6,10*]henicosa-1(20),6,8, 10(21),16,18-hexaen-2-one

The title compound is obtained by an analogous reaction sequence as for example 35, starting from acetic acid tert-butoxycarbonylamino-[(R)-3-(3-isopropyl-benzyl)-2-oxo-oxazolidin-5-yl]-methyl ester (building block C9) and 2-allyl-4-chloromethyl-1-fluoro-benzene (building block F5), and further 2-(acetyl-allyl-amino)-6-methyl-isonicotinic acid (building block A44) instead of 2-(acetyl-allyl-amino)-6-chloro-isonicotinic acid (building block A27).

LC (Zorbax SB-C18H, 3×30 mm, 1.8 μm, 30-100% MeCN (3.25 min), 100% MeCN (0.75 min), 100-30% MeCN (0.25 min)): 1.998 min

MS (ES+): 519=[M+H]⁺

### Example 67

### (S)-4-{(R)-2-[(4-tert-Butyl-pyridin-2-ylmethyl)-amino]-1-hydroxy-ethyl}-18-oxazol-2-yl-3,15-diaza-tricyclo[14.3.1.1*6,10*]henicosa-1(19),6,8,10(21),16(20),17-hexa-en-2-one

The title compound is obtained by an analogous reaction sequence as for example 7, starting from {(1S,2R)-1-(3-allyl-benzyl)-3-[benzyloxycarbonyl-(4-tert-butyl-pyridin-2-ylmethyl)-amino]-2-hydroxy-propyl}-carbamic acid tert-butyl ester (building block C14) and 3-(allyl-benzyloxycarbonyl-amino)-5-oxazol-2-yl-benzoic acid (building block A17).

MS (ES+): 568=[M+H]⁺

¹H-NMR (400 MHz, d6-DMSO): 8.42 (d, 1H), 8.14 (s, 1H), 8.10 (d, 1H), 7.47 (s, 1H), 7.39 (s, 1H), 7.31 (s, 1H), 7.30-7.28 (m, 1H), 7.20 (s, 2H), 7.16 (t, 1H), 7.05 (d, 1H), 7.00 (d, 1H), 6.39 (s, 1H), 6.32 (t, 1H), 5.33 (br s, 1H), 4.1-4.0 (m, 2H), 3.71 (br s, 1H), 3.4-3.3 (m, 1H), 3.16 (dd, 1H), 3.08-3.62 (m, 6H), 1.98-1.88 (m, 1H), 1.82-1.72 (m, 1H), 1.51-1.39 (m, 1H), 1.26 (s, 9H), 1.18-1.08 (m, 1H).

### Example 68

### (S)-4-{(R)-1-Hydroxy-2-[(4-isopropyl-pyridin-2-ylmethyl)-amino]-ethyl}-19-methyl-11-oxa-3,16,18-triaza-tricyclo[15.3.1.1*6,10*]docosa-1(21),6,8,10(22),17,19-hexa-en-2-one

### a) (S)-16-Acetyl-4-((R)-1-hydroxy-2-[(4-isopropyl-pyridin-2-ylmethyl)-amino]-ethyl}-19-methyl-11-oxa-3,16,18-triaza-tricyclo[15.3.1.1*6,10*]docosa-1(21),6,8,10(22),17,19-hexaen-2-one

The title compound is obtained by an analogous reaction sequence as for example 7, starting from {(1S,2R)-1-(3-allyloxy-benzyl)-3-[benzyloxycarbonyl-(4-isopropyl-pyridin-2-ylmethyl)-amino]-2-hydroxy-propyl}-carbamic acid tert-butyl ester (building block C7) and 2-(acetyl-allyl-amino)-6-methyl-isonicotinic acid (building block A44).

MS (ES+): 560=[M+H]⁺

HPLC (Zorbax SB-C18, 3×30 mm, 1.8 μm, 0-100% AcCN (3.25 min), 100% AcCN (0.75 min)) retention time=3.04 min

### b) (S)-4-{(R)-1-Hydroxy-2-[(4-isopropyl-pyridin-2-ylmethyl)-amino]-ethyl}-19-methyl-11-oxa-3,16,18-triaza-tricyclo[15.3.1.1*6,10*]docosa-1(21),6,8,10(22),17,19-hexaen-2-one

(S)-16-Acetyl-4-{(R)-1-hydroxy-2-[(4-isopropyl-pyridin-2-ylmethyl)-amino]-ethyl}-19-methyl-11-oxa-3,16,18-triaza-tricyclo[15.3.1.1*6,10*]docosa-1(21),6,8,10(22),17,19-hexaen-2-one (103 mg, 0.18 mmol) are stirred for 5 h at 60° C. in a mixture of EtOH (8 ml) and 2N aq. NaOH (0.92 ml, 10 eq). After cooling to rt the mixture is diluted with water and extracted with DCM. The extracts are dried over magnesium sulfate and the solvents are evaporated. The residue is purified by crystallization from DCM/MeOH 9/1 and a little Et₂O and hexane to give the product.

MS (ES+): 518=[M+H]⁺

¹H-NMR (400 MHz, d6-DMSO): 8.39 (d, 1H), 8.13 (d, 1H), 7.30 (s, 1H), 7.20-7.13 (m, 2H), 7.03 (s, 1H), 6.82 (d, 1H), 6.77 (d, 1H), 6.56 (t, 1H), 6.34 (s, 1H), 6.17 (s, 1H), 5.04 (br s, 1H), 4.28-4.22 (m, 1H), 3.97-3.83 (m, 2H), 3.80 (s, 2H), 3.60-3.50 (m, 1H), 3.05-2.96 (m, 2H), 2.92-2.82 (m, 1H), 2.69-2.63 (m, 2H), 2.57-2.52 (m, 2H), 2.22 (s, 3H), 1.8-1.6 (m, 3H), 1.55-1.45 (m, 1H), 1.20 (d, 6H).

### Example 69

### (S)-4-{(R)-2-[(4-tert-Butyl-pyridin-2-ylmethyl)-amino]-1-hydroxy-ethyl}-18-(2-oxo-propoxy)-15-oxa-3-aza-tricyclo[14.3.1.1*6,10*]henicosa-1(19),6,8,10(21),-16(20),17-hexaen-2-one

The title compound is obtained by an analogous reaction sequence as for example 7, starting from {(1S,2R)-1-(3-allyl-benzyl)-3-[benzyloxycarbonyl-(4-tert-butyl-pyridin-2-ylmethyl)-amino]-2-hydroxy-propyl}-carbamic acid tert-butyl ester (building block C14) and 3-allyloxy-5-(2-oxo-propoxy)-benzoic acid (building block A21).

MS (ES+): 574=[M+H]⁺

¹H-NMR (400 MHz, d6-DMSO): 8.83 (d, 1H), 7.97 (d, 1H), 7.44 (s, 1H), 7.38 (s, 1H), 7.24 (d, 1H), 7.13 (t, 1H), 7.03 (d, 1H), 6.97 (d, 1H), 6.55 (s, 1H), 6.38-6.35 (m, 2H), 5.03 (br s, 1H), 4.75 (s, 2H), 4.30 (dt, 1H), 4.12-4.01 (m, 2H), 3.84 (s, 2H), 3.7-3.6 (m, 1H), 3.11 (dd, 1H), 2.74-2.57 (m, 5H), 2.10 (s, 3H), 1.96-1.76 (m, 2H), 1.55-1.30 (m, 2H), 1.27 (s, 9H).

### Example 70

### (S)-4-{(R)-1-Hydroxy-2-[(4-isopropyl-pyridin-2-ylmethyl)-amino]-ethyl}-19-(2-oxo-propoxy)-11,16-dioxa-3-aza-tricyclo[15.3.1.1*6,10*]docosa-1(21),6,8,10(22),-17,19-hexaen-2-one

The title compound is obtained by an analogous reaction sequence as for example 7, starting from {(1S,2R)-1-(3-allyloxy-benzyl)-3-[benzyloxycarbonyl-(4-isopropyl-pyridin-2-ylmethyl)-amino]-2-hydroxy-propyl}-carbamic acid tert-butyl ester (building block C7) and 3-allyloxy-5-(2-oxo-propoxy)-benzoic acid (building block A21).

MS (ES+): 576=[M+H]⁺

¹H-NMR (400 MHz, d6-DMSO): 8.39 (d, 1H), 8.05 (d, 1H), 7.30 (s, 1H), 7.18 (t, 1H), 7.14 (d, 1H), 7.02 (s, 1H), 6.84 (d, 1H), 6.77 (d, 1H), 6.69 (s, 1H), 6.61 (s, 1H), 6.49 (s, 1H), 5.09 (br s, 1H), 4.81 (s, 2H), 4.39-4.33 (m 1H), 4.28-4.21 (m, 1H), 4.12-3.88 (m, 3H), 3.82 (s, 2H), 3.66-3.61 (m, 1H), 2.99 (dd, 1H), 2.92-2.81 (m, 1H), 2.75-2.69 (m, 2H), 2.61-2.53 (m, 1H), 2.14 (s, 3H), 1.86-1.66 (m, 4H), 1.20 (d, 6H), 1.20-1.12 (m, 1H).

### Example 71

### (S)-4-{(R)-1-Hydroxy-2-[(4-isopropyl-pyridin-2-ylmethyl)-amino]-ethyl}-18-(2-oxo-propoxy)-15-oxa-3-aza-tricyclo[14.3.1.1*6,10*]henicosa-1(20),6,8,10(21),16,18-hexaen-2-one

The title compound is obtained by an analogous reaction sequence as for example 7, starting from {(1S,2R)-1-(3-allyl-benzyl)-3-[benzyloxycarbonyl-(4-isopropyl-pyridin-2-ylmethyl)-amino]-2-hydroxy-propyl}-carbamic acid tert-butyl ester (building block C8) and 3-allyloxy-5-(2-oxo-propoxy)-benzoic acid (building block A21).

MS (ES+): 560=[M+H]³⁰

¹H-NMR (400 MHz, d6-DMSO): 8.40 (d, 1H), 8.01 (d, 1H), 7.41 (s, 1H), 7.34 (s, 1H), 7.18-7.13 (m, 2H), 7.07 (d, 1H), 7.00 (d, 1H), 6.58 (s, 1H), 6.41 (s, 1H), 6.38 (s, 1H), 5.03 (d, 1H), 4.78 (s, 2H), 4.35-4.28 (m, 1H), 4.15-4.05 (m, 2H), 3.83 (s, 2H), 3.69-3.64 (m, 1H), 3.12 (dd, 1H), 2.94-2.83 (m, 1H), 2.76-2.59 (m, 4H), 2.11 (s, 2H), 1.98-1.77 (m, 2H), 1.53-1.33 (m, 3H), 1.21 (d, 6H), 1.2-1.13 (m, 1H).

### Example 72

### (S)-4-[(R)-2-[(3-tert-Butyl-benzylamino)-1-hydroxy-ethyl]-2-oxo-11,16-dioxa-3-aza-tricyclo[15.3.1.1*6,10*]docosa-1(21),6,8,10(22),17,19-hexaene-19-carboxylic acid dimethylamide

The title compound is obtained by an analogous reaction sequence as for example 43, starting from (S)-4-(S)-oxiranyl-2-oxo-11,16-dioxa-3-aza-tricyclo[15.3.1.1*6,10*]docosa-1(21),6,8,10(22),17,19-hexaene-19-carboxylic acid dimethylamide (building block E1) and 3-tert-butyl-benzylamine (building block D4).

Pale yellow solid.

Rf: (DCM/MeOH=90/10): 0.31

MS (ES+): 588=[M+H]⁺

¹H-NMR (400 MHz, d6-DMSO): 8.12 (d, 1H), 7.32 (s, 1H), 7.21-7.09 (m, 4H), 7.05 (s, 1H), 7.00-6.97 (m, 2H), 6.93-6.91 (m, 1H), 6.83-6.79 (m, 1H), 6.76-6.72 (m, 1H), 4.97 (br, 1H), 4.42-4.34 (m, 1H), 4.26-4.19 (m, 1H), 4.16-4.09 (m, 1H), 4.01-3.88 (m, 2H), 3.71 (s, 2H), 3.64-3.58 (m, 1H), 2.96 (s, 3H), 2.84 (s, 3H), 2.69-2.64 (m, 1H), 1.88-1.64 (m, 3H), 1.25 (s, 9H).

### Example 73

### (E)-(S)-4-{(R)-2-[(4-tert-Butyl-pyridin-2-ylmethyl)-amino]-1-hydroxy-ethyl}-19-chloro-11-oxa-3,16,18-triaza-tricyclo[15.3.1.1*6,10*]docosa-1(21),6,8,10(22),13,17,19-heptaen-2-one

### a) 2-(Acetyl-allyl-amino)-N—[(S)-2-(3-allyloxy-phenyl)-1-((R)-2-oxo-oxazolidin-5-yl)-ethyl]-6-chloro-isonicotinamide

The compound is obtained by an analogous method as for example 35a, starting from [(S)-2-(3-allyloxy-phenyl)-1-((R)-2-oxo-oxazolidin-5-yl)-ethyl]-carbamic acid tert-butyl ester (building block C18) and 2-(acetyl-allyl-amino)-6-chloro-isonicotinic acid (building block A27).

Rf: (EtOAc/toluene=2:1): 0.14

LC (Zorbax SB-C18H, 3×30 mm, 1.8 μm, 30-100% AcCN (3.25 min), 100% AcCN (0.75 min), 100-30% AcCN (0.25 min)): 2.533 min

MS (ES+): 499, 501=[M+H]⁺

### b) (E)-(S)-16-Acetyl-19-chloro-4-((R)-2-oxo-oxazolidin-5-yl)-11-oxa-3,16,18-triaza-tricyclo[15.3.1.1*6,10*]docosa-1(21),6,8,10(22),13,17,19-heptaen-2-one

To an at 45° C. stirred solution of 1.29 g (2.59 mmol) 2-(acetyl-allyl-amino)-N—[(S)-2-(3-allyloxy-phenyl)-1-((R)-2-oxo-oxazolidin-5-yl)-ethyl]-6-chloro-isonicotinamide in DCM is added 100 mg of Grubbs II catalyst. After refluxing for 3 h, the mixture is cooled down and quenched with 0.2 ml butyl vinyl ether and 0.5 g activated charcoal. The mixture is purified by chromatography on silica gel (toluene/EtOAc=: 1, 1:3; EtOAc) to give the product in the form of a grey foam.

Rf: (EtOAc): 0.16

LC (Zorbax SB-C18H, 3×30 mm, 1.8 μm, 30-100% AcCN (3.25 min), 100% AcCN (0.75 min), 100-30% AcCN (0.25 min)): 0.937 min

MS (ES+): 471, 473=[M+H]⁺

### c) (R)-5-((E)-(S)-16-Acetyl-19-chloro-2-oxo-11-oxa-3,16,18-triaza-tricyclo[15.3.1.1*6,10*]docosa-1(21),6,8,10(22),13,17,19-heptaen-4-yl)-2-oxo-oxazolidine-3-carboxylic acid tert-butyl ester

To a solution of 971 mg (1.95 mmol) (E)-(S)-16-acetyl-19-chloro-4-((R)-2-oxo-oxazolidin-5-yl)-11-oxa-3,16,18-triaza-tricyclo[15.3.1.1*6,10*]docosa-1(21),6,8,10(22),13,17,19-heptaen-2-one in 20 ml DCM is added 47 mg (0.4 mmol) DMAP, 0.42 ml (3 mmol) triethyl amine and 477 mg (2.14 mmol) tert-butyl-pyrocarbonate. After 2 h the mixture is diluted with EtOAc, washed with 5% citric acid and water. Purification by chromatography on silica gel (EtOAc/hexane=2:1) gives the product in the form of a yellow foam.

Rf: (EtOAc): 0.68

LC (Zorbax SB-C18H, 3×30 mm, 1.8 μm, 30-100% AcCN (3.25 min), 100% AcCN (0.75 min), 100-30% AcCN (0.25 min)): 2.867 min

MS (ES+): 1165, 1167, 1169=[2M+Na]⁺

### d) [(R)-2-((E)-(S)-19-Chloro-2-oxo-11-oxa-3,16,18-triaza-tricyclo[15.3.1.1*6,10*]docosa-1(21),6,8,10(22),13,17,19-heptaen-4-yl)-2-hydroxy-ethyl]-carbamic acid tert-butyl ester

A solution of 990 mg (1.73 mmol) (R)-5-((E)-(S)-16-acetyl-19-chloro-2-oxo-11-oxa-3,16,18-triaza-tricyclo[15.3.1.1*6,10*]docosa-1(21),6,8,10(22),13,17,19-heptaen-4-yl)-2-oxo-oxazolidine-3-carboxylic acid tert-butyl ester in 20 ml MeOH is treated with 338 mg (1.04 mmol) cesium carbonate and stirred overnight. The mixture is dilute with 1 ml DMF and 20 ml EtOAc. The mixture is washed with water, dried over sodium sulfate and evaporated to yield a white powder.

Rf: (EtOAc): 0.72

LC (Zorbax SB-C18H, 3×30 mm, 1.8 μm, 30-100% AcCN (3.25 min), 100% AcCN (0.75 min), 100-30% AcCN (0.25 min)): 2.539 min

MS (ES+): 503, 505=[M+H]⁺

### e) (E)-(S)-4-((R)-2-Amino-1-hydroxy-ethyl)-19-chloro-11-oxa-3,16,18-triaza-tricyclo[15.3.1.1*6,10*]docosa-1(21),6,8,10(22),13,17,19-heptaen-2-one

A suspension of 255 mg (0.45 mmol) [(R)-2-((E)-(S)-19-chloro-2-oxo-11-oxa-3,16,18-triaza-tricyclo[15.3.1.1*6,10*]docosa-1(21),6,8,10(22),13,17,19-heptaen-4-yl)-2-hydroxy-ethyl]-carbamic acid tert-butyl ester is stirred at 50° C. for 1 h. The mixture is evaporated and 3 ml 10% aq. Na₂CO₃ is added. The mixture is diluted with 50 ml THF, dried over K₂CO₃/Na₂SO₄ and purified by chromatography on silica gel (DCM/MeOH=19:1, DCM/MeOH/25% aq. NH₃=19:1:0.1, DCM/MeOH/25% aq. NH₃=9:1:01 and DCM/MeOH/25% aq. NH₃=6:1:01) to give the product in the form of a white powder.

LC (Zorbax SB-C18H, 3×30 mm, 1.8 μm, 10-100% AcCN (3.25 min), 100% AcCN (0.75 min), 100-30% AcCN (0.25 min)): 2.539 min

MS (ES+): 403, 405=[M+H]⁺

### f) (E)-(S)-4-((R)-2-[(4-tert-Butyl-pyridin-2-ylmethyl)-amino]-1-hydroxy-ethyl}-19-chloro-11-oxa-3,16,18-triaza-tricyclo[15.3.1.1*6,10*]docosa-1(21),6,8,10(22),13,17,19-heptaen-2-one

A solution of 105 mg (0.26 mmol) (E)-(S)-4-((R)-2-Amino-1-hydroxy-ethyl)-19-chloro-11-oxa-3,16,18-triaza-tricyclo[15.3.1.1*6,10*]docosa-1(21),6,8,10(22),13,17,19-heptaen-2-one in 10 ml THF/EtOH (1:1) is treated with 43 mg (0.26 mmol) 4-tert-butyl-pyridine-2-carbaldehyde (building block D6) and slowly concentrated under slightly reduced pressure at 50° C. This is repeated with another 10 ml THF/EtOH (1:1). The residue is taken up in 2 ml EtOH and treated with 38 mg (1 mmol) NaBH₄. After 2 h the mixture is quenched with 1 ml 2N HCl. After 1 h the mixture is evaporated, and the residue is chromatographed on silica gel (DCM/MeOH=20:1 and DCM/MeOH/25% aqNH₃=19:1:0.1) to give the product in the form of a white solid.

Rf: (DCM/MeOH/25% aqNH₃=9:1:0.1): 0.35

LC (Nucleosil C-18HD, 4×70 mm, 3 μm, 20-100% AcCN (6 min), 100% AcCN (1.5 min)): 4.012 min

MS (ES+): 550=[M+H]⁺

### Example 74

### (S)-4-{(R)-2-[(4-tert-Butyl-pyridin-2-ylmethyl)-amino]-1-hydroxy-ethyl}-19-chloro-11-oxa-3,16,18-triaza-tricyclo[15.3.1.1*6,10*]docosa-1(21),6,8,10(22),17,19-hexaen-2-one

A solution of 114 mg (0.20 mmol) (E)-(S)-4-{(R)-2-[(4-tert-butyl-pyridin-2-ylmethyl)-amino]-1-hydroxy-ethyl}-19-chloro-11-oxa-3,16,18-triaza-tricyclo[15.3.1.1*6,10*]docosa-1(21),6,8,10(22), 13,17,19-heptaen-2-one (example 73) in 5 ml THF/EtOH (1:1) is hydrogenated 16 h in the presence of 200 mg Raney-Ni and at 1 atm hydrogen pressure. The mixture is filtered over celite, concentrated and chromatographed on RP-18 reverse phase silica gel using AcCN/water (0.1% TFA, gradient from 70 to 30% water). The fractions containing product are concentrated, basified with solid Na₂CO₃ and extracted with THF. The organic phase is dried over potassium carbonate and lyophilized with tert-butanol to give the product in the form of a white powder.

LC (Nucleosil C-18HD, 4×70 mm, 3 μm, 20-100% AcCN (6 min), 100% AcCN (1.5 min)): 4.100 min

MS (ES+): 552=[M+H]⁺

### Example 75

### (S)-4-[(R)-1-Hydroxy-2-(3-isopropyl-benzylamino)-ethyl]-18-methyl-11-oxa-3,15,17-triaza-tricyclo[14.3.1.1*6,10*]henicosa-1(20),6,8,10(21),16,18-hexaen-2-one

### a) {(S)-2-[3-(3-Hydroxy-propoxy)-phenyl]-1-[(R)-3-(3-isopropyl-benzyl)-2-oxo-oxazolidin-5-yl]-ethyl}-carbamic acid tert-butyl ester

A mixture of 1.03 g (2.28 mmol) {(S)-2-(3-hydroxy-phenyl)-1-[(R)-3-(3-isopropyl-benzyl)-2-oxo-oxazolidin-5-yl]-ethyl}-carbamic acid tert-butyl ester (building block C16), 0.63 g (4.6 mmol) potassium carbonate and 0.3 ml (3.41 mmol) 3-bromo-1-propanol in 3 ml DMF is stirred at 50° C. for 18 h. The reaction mixture is cooled down, diluted with 10 ml water and extracted with ethyl acetate. The organic phase is washed with water, dried over sodium sulfate and concentrated in vacuo. Chromatography on silica gel (EtOAc/hexane 1:1) gives the title compound in the form of a colorless resin.

Rf (EtOAc/hexanes)=1/1): 0.23

LC (Nucleosil C-18HD, 4×70 mm, 3 μm, 20-100% AcCN (6 min), 100% AcCN (1.5 min)): 5.527 min

MS (ES+): 1047=[2M+Na]⁺

### b) Methanesulfonic acid 3-(3-((S)-2-tert-butoxycarbonylamino-2-[(R)-3-(3-isopropyl-benzyl)-2-oxo-oxazolidin-5-yl]-ethyl}-phenoxy)-propyl ester

Methane sulfonyl chloride (0.228 ml, 2.91 mmol) is added dropwise to a stirred solution of 1.15 g (2.24 mmol) {(S)-2-[3-(3-hydroxy-propoxy)-phenyl]-1-[(R)-3-(3-isopropyl-benzyl)-2-oxo-oxazolidin-5-yl]-ethyl}-carbamic acid tert-butyl ester and 0.62 ml (4.5 mmol) triethylamine in 10 ml dry THF. After 6 h the reaction mixture is quenched with water and extracted with ethyl acetate. The organic phase is washed with water, dried over sodium sulfate and concentrated in vacuo. Chromatography on silica gel (EtOAc/hexanes 1:1) gives the title compound in the form of a colorless resin.

Rf: (EtOAc/hexanes)=1/1): 0.28

LC (Nucleosil C-18HD, 4×70 mm, 3 μm, 20-100% AcCN (6 min), 100% AcCN (1.5 min)): 5.905 min

MS (ES+): 1203=[2M+Na]⁺

### c) 2-[(Benzyloxycarbonyl-[3-(3-{(S)-2-tert-butoxycarbonylamino-2-[(R)-3-(3-isopropyl-benzyl)-2-oxo-oxazoldin-5-yl]-ethyl}-phenoxy)-propyl]-amino}-6-methyl-isonicotinic acid ethyl ester

A mixture of 1.15 g (1.95 mmol) methanesulfonic acid 3-(3-{(S)-2-tert-butoxycarbonylamino-2-[(R)-3-(3-isopropyl-benzyl)-2-oxo-oxazolidin-5-yl]-ethyl}-phenoxy)-propyl ester, 0.612 g (1.95 mmol) 2-benzyloxycarbonylamino-6-methyl-isonicotinic acid ethyl ester (building block A43), 0.293 mg (1.95 mmol) sodium iodide and 0.763 (2.34 mmol) caesium carbonate in 2 ml DMF is stirred at rt for 2 d. The mixture is diluted with water and extracted with ethyl acetate. The organic phase is washed with water, dried over sodium sulfate and concentrated. Chromatography on silica gel (EtOAc/hexanes 1:3) gives the title compound in the form of a colorless resin.

Rf: (EtOAc/hexanes)=1/3): 0.13

LC (Nucleosil C-18HD, 4×70 mm, 3 μm, 20-100% AcCN (6 min), 100% AcCN (1.5 min)): 7.342 min

MS (ES+): 809=[M+H]⁺

### d) (S)-4-[(R)-3-(3-Isopropyl-benzyl)-2-oxo-oxazolidin-5-yl]-18-methyl-2-oxo-1-oxa-3,15,17-triaza-tricyclo[14.3.1.1*6,10*]henicosa-1(20),6,8,10(21),16,18-hexaene-15-carboxylic acid benzyl ester

A solution of 250 mg (0.31 mmol) 2-{benzyloxycarbonyl-[3-(3-{(S)-2-tert-butoxycarbonyl-amino-2-[(R)-3-(3-isopropyl-benzyl)-2-oxo-oxazolidin-5-yl-ethyl}-phenoxy)-propyl]-amino}-6-methyl-isonicotinic acid ethyl ester in 5 ml MeOH is treated with 1.5 ml 1N NaOH. After 1.5 h the mixture is evaporated and taken up in 5 ml 4N HCl in dioxane and stirred for 2 h at rt. The mixture is evaporated again and under ice-cooling the residue is suspended in 20 ml DCM and treated successively with 70 mg (0.46 mmol) HOBt, 89 mg (0.46 mmol) EDC and finally with 0.171 ml N-methylmorpholine. After stirring for 18 h at rt the mixture is washed with water, 5% citric acid, 5% NaHCO₃ solution and water. The organic phase is dried over sodium sulfate and concentrated. The residue is purified by chromatography on silica gel (EtOAc/hexane 1:1) to give the product in the form of a colorless resin.

Rf: (EtOAc/hexanes)=1/1): 0.15

LC (Nucleosil C-18HD, 4×70 mm, 3 μm, 20-100% AcCN (6 min), 100% AcCN (1.5 min)): 5.887 min

MS (ES+): 663=[M+H]⁺

### e) (S)-4-[(R)-3-(3-Isopropyl-benzyl)-2-oxo-oxazolidin-5-yl]-18-methyl-11-oxa-3,15,17-triaza-tricyclo[14.3.1.1*6,10*]henicosa-1(20),6,8,10(21),16,18-hexaen-2-one

A solution of 150 mg (0.23 mmol) (S)-4-[(R)-3-(3-isopropyl-benzyl)-2-oxo-oxazolidin-5-yl]-18-methyl-2-oxo-11-oxa-3, 15,17-triaza-tricyclo[14.3.1.1*6,10*]henicosa-1(20),6,8, 10(21), 16,18-hexaene-15-carboxylic acid benzyl ester in 10 ml ethanol/THF(1/1) is hydrogenated (1 atm H₂) at rt with 50 mg Pd/C (10% Engelhard 4505) for 3 h. Filtration through celite and evaporation of the solvent followed by chromatography on silica (Ee/hexane 1:1) gives the product in the form of a white solid.

LC (Nucleosil C-18HD, 4×70 mm, 3 μm, 20-100% AcCN (6 min), 100% AcCN (1.5 min)): 4.259 min

MS (ES+): 5.29=[M+H]⁺

### f) (E)-(S)-4-{(R)-2-[(4-tert-Butyl-pyridin-2-ylmethyl)-amino]-1-hydroxy-ethyl}-19-chloro-11-oxa-3,16,18-triaza-tricyclo[15.3.1.1*6,10*]docosa-1(21),6,8,10(22),13,17,19-heptaen-2-one

A mixture of 100 mg (0.19 mmol) (S)-4-[(R)-3-(3-isopropyl-benzyl)-2-oxo-oxazolidin-5-yl]-18-methyl-11-oxa-3, 15,17-triaza-tricyclo[14.3.1.1*6,10*]henicosa-1(20),6,8,10(21),16,18-hexaen-2-one and 97 mg (0.57 mmol) barium hydroxide in 4 ml EtOH/water (1/1) is heated at 80° C. for 2 days. The mixture is filtered over celite, washed with water and THF. The filtrate is diluted with a 10% solution of sodium carbonate in water and extracted with THF. The organic phase is dried over sodium sulfate, concentrated and purified by chromatography on silica gel (DCM/MeOH=95/5 and DCM/MeOH/255 aq. NH₃=95/5/0.5) to give the product in the form of a slightly yellow foam.

Rf: (DCM/MeOH/25% aq NH3=90/9/1): 0.45

LC (Nucleosil C-18HD, 4×70 mm, 3 μm, 20-100% AcCN (6 min), 100% AcCN (1.5 min)): 3.152 min

MS (ES+): 503=[M+H]⁺

### Building Blocks

### Building block A1: 2-But-3-enylamino-6-methyl-isonicotinic acid hydrochloride

### a) 2-Chloro-6-methyl-isonicotinic acid tert-butyl ester

A solution of 5.0 g (29 mmol) 2-Chloro-6-methyl-isonicotinic acid in 50 ml chloroform is heated to reflux temperature. 14 ml (58 mmol, 2 eq) Di-tert-butoxymethyl-dimethyl-amine are added dropwise over 30 min. After 1.5 h and 3.5 h reaction time another portion of di-tert-butoxymethyl-dimethyl-amine (each time 14 ml, 58 mmol, 2 eq) is added. After 4.5 h the reaction mixture is cooled to rt, diluted with EtOAc, washed with aqueous bicarbonate and brine and dried over magnesium sulfate. The product is purified by chromatography on silica (flashmaster, hexane to hexane/EtOAc 95/5) to give a white solid.

Rf: (DCM/methanol=95:5): 0.36

MS (LC/MS): 172/174=[M+H-tBu]⁺

¹H-NMR (400 MHz, CDCl₃): 7.65 (s, 1H), 7.60 (s, 1H), 2.63 (s, 3H), 1.63 (s, 9H).

### b) 2-But-3-enylamino-6-methyl-isonicotinic acid tert-butyl ester

To a solution of 1.57 g (16.3 mmol, 2.4 eq) sodium tert-butylate in 20 ml dry dioxane are added under an atmosphere of argon 1.55 g (6.8 mmol) 2-Chloro-6-methyl-isonicotinic acid tert-butyl ester and 0.90 g (8.2 mmol, 1.2 eq) but-3-enylamine hydrochloride. The mixture is stirred at rt for 30 min and then heated to 80° C. 83 mg (0.02 eq) of the catalyst SK-CC02-A are added as a solution in 6 ml dioxane. The reaction mixture is stirred at 110° C. for 18 h. After cooling to rt the reaction is diluted with EtOAc, washed with aqueous bicarbonate and brine, and dried over magnesium sulfate. The solvents are evaporated at reduced pressure and the residue is purified by chromatography on silica (flashmaster, hexane to hexane/EtOAc 85/15) to give the product in the form of a yellow oil.

Rf: (Hexane/EtOAc=90/10): 0.18

MS (LC/MS): 263=[M+H]⁺

¹H-NMR (400 MHz, CDCl₃): 6.95 (s, 1H), 6.75 (s, 1H), 5.91-5.81 (m, 1H), 5.21-5.13 (m, 2H), 4.71 (br s, 1H), 3.41-3.36 (m, 2H), 2.45 (s, 3H), 2.45-2.40 (m, 2H), 1.62 (s, 9H).

### c) 2-But-3-enylamino-6-methyl-isonicotinic acid hydrochloride

A solution of 0.59 g (2.2 mmol) 2-But-3-enylamino-6-methyl-isonicotinic acid tert-butyl ester in 8.4 ml (15 eq) 4 N HCl in dioxane is heated over night at 60° C. Evaporation of the solvent and trituration with ether gives the product in the form of a brownish foam.

Rf: (DCM/MeOH=70/30): 0.37

MS (LC/MS): 207=[M+H]⁺

¹H-NMR (400 MHz, CDCl₃): 8.52 (br s, 1H), 7.27 (s, 1H), 7.07 (s, 1H), 5.9-5.8 (m, 1H), 5.28-5.19 (m, 2H), 3.48 (br s, 2H), 2.66 (s, 3H), 2.52 (br d, 2H).

### Building block A2: 2-Allylamino-6-methyl-isonicotinic acid hydrochloride

### a) 2-Allylamino-6-methyl-isonicotinic acid tert-butyl ester

A mixture of 1.87 ml (24 mmol, 1.1 eq) allylamine, 0.254 g (0.05 eq) Pd(OAc)₂, 0.705 g (0.05 eq) BINAP and 4.78 g (48 mmol, 2.2 eq) sodium tert-butylate in 110 ml toluene is heated to 60° C. under an atmosphere of nitrogen. 5.0 g (22 mmol) 2-Chloro-6-methyl-isonicotinic acid tert-butyl ester (see building block A1) dissolved in 40 ml toluene is added dropwise over 30 min., and the reaction is stirred at 60° C. for another 2.5 h. After cooling to rt the reaction is diluted with EtOAc, washed with aqueous bicarbonate and brine, and dried over sodium sulfate. The solvents are evaporated at reduced pressure and the residue is purified by chromatography on silica (flashmaster, hexane to hexane/EtOAc 80/20) to give the product.

Rf: (hexane/EtOAc=70/30): 0.38.

MS (LC/MS): 193=[M+H-tBu]+

¹H-NMR (400 MHz, CDCl₃): 6.96 (s, 1H), 6.76 (s, 1H), 6.02-5.93 (m, 1H), 5.31 (d, 1H), 5.20 (d, 1H), 4.81 (br s, 1H), 4.00-3.95 (m, 2H), 2.45 (s, 3H), 1.61 (s, 9H).

### b) 2-Allylamino-6-methyl-isonicotinic acid hydrochloride

A solution of 0.26 g (1.0 mmol) 2-Allylamino-6-methyl-isonicotinic acid tert-butyl ester in 9.2 ml (35 eq) 4 N HCl in dioxane is heated for 2 h at 60° C. Evaporation of the solvent gives the product in the form of a brownish foam.

Rf: (DCM/MeOH=70/30): 0.37

MS (LC/MS): 193=[M+H]

¹H-NMR (400 MHz, CDCl₃): 8.61 (br s, 1H), 7.24 (s, 1H), 7.10 (s, 1H), 5.95-5.86 (m, 1H), 5.39 (d, 1H), 5.33 (d, 1H), 4.07 (br s, 2H), 2.68 (s, 3H).

### Building block A3: 2-But-3-enylamino-6-methoxy-isonicotinic acid

### a) 2-Chloro-6-methoxy-isonicotinic acid

To a solution of 13.6 g (102 mmol, 2 eq) sodium hydroxide in 50 ml MeOH 10.0 g (51 mmol, 1 eq) 2,6-dichloro-isonicotinic acid are added. The reaction mixture is heated at reflux temperature for 30 min. After cooling to rt the mixture is diluted with water and acidified to pH 5-6 with 4 N HCl. Extraction with EtOAc, drying over sodium sulfate and evaporation of the solvent gives a first amount of the product. The water layers are evaporated to about 250 ml, acidified with 4 N HCl to pH 4-5 and again extracted with EtOAc. The extracts are dried over sodium sulfate and evaporated to give a further amount of the product. The product contains traces of 2,6-dimethoxy-isonicotinic acid.

MS (LC/MS): 188/190=[M+H]⁺

¹H-NMR (400 MHz, d6-DMSO): 7.38 (s, 1H), 7.16 (s, 1H), 3.90 (s, 3H).

### b) 2-But-3-enylamino-6-methoxy-isonicotinic acid

To a solution of 3.87 g (53 mmol, 10 eq) but-3-enylamine hydrochloride in 13.3 ml (53 mmol, 10 eq) 4 N aqueous sodium hydroxide 1.0 g (5.3 mmol, 1 eq) 2-Chloro-6-methoxy-isonicotinic acid and 1.33 g (5.3 mmol, 1 eq) copper(II)sulfate pentahydrate are added and the reaction is heated in a closed vessel during 18 h at a bath temperature of 160° C. After cooling to rt the mixture is diluted 400 ml aqueous 10% citric acid and extracted with EtOAc. The extracts are washed with water and brine, dried over sodium sulfate and the solvents are evaporated at reduced pressure. The residue is dissolved in hot DCM/MeOH. After cooling the inorganic precipitates are filtered off (repeated twice) to give the crude product. This is stirred in water, filtered off and dried in vacuum. A further amount of the product is obtained from the residue of the evaporated washing water by chromatography on silica (short column, flashmaster, DCM to DCM/MeOH 85/15).

MS (LC/MS): 223=[M+H]⁺

¹H-NMR (400 MHz, CDCl₃): 6.90 (t, 1H), 6.53 (s, 1H), 6.24 (s, 1H), 5.92-5.82 (m, 1H), 5.10 (d, 1H), 5.04 (d, 1H), 3.81 (s, 3H), 3.35-3.30 (m, 2H), 2.34-2.29 (m, 2H).

### Building block A4: 2-Allylamino-6-methoxy-isonicotinic acid

A mixture of 3.97 ml (52 mmol, 10 eq) allylamine, 0.97 g (5.2 mmol, 1 eq) 2-chloro-6-methoxy-isonicotinic acid (see building block A3) and 1.29 g (5.2 mmol, 1 eq) copper(II)sulfate pentahydrate in 10 ml water is heated in a closed vessel during 2.5 h at a bath temperature of 160° C. After cooling to rt the mixture is diluted with 400 ml aqueous 10% citric acid and extracted with EtOAc. The extracts are washed with water and brine, dried over sodium sulfate and the solvents are evaporated at reduced pressure. The residue is dissolved in about 20 ml hot DCM/MeOH (2/1) On addition of about 25 ml of hexane, partial evaporation of the solvents to a volume of about 20 ml and keeping at 4° C. for 4 h the product precipitates and is filtered off and dried in vacuum.

MS (LC/MS): 209=[M+H]⁺

¹H-NMR (400 MHz, d6-DMSO): 7.04 (t, 1H), 6.55 (s, 1H), 6.26 (s, 1H), 5.97-5.88 (m, 1H), 5.21 (d, 1H), 5.09 (d, 1H), 3.92 (d, 2H), 3.80 (s, 3H).

### Building block A5: 2-(Allyl-methyl-amino)-6-methoxy-isonicotinic acid hydrochloride

### a) 2-Chloro-6-methoxy-isonicotinic acid tert-butyl ester

A solution of 10.0 g (53.3 mmol) 2-Chloro-6-methoxy-isonicotinic acid in 60 ml DMF is heated to 100° C. for 4 h, then to 80° C. for another 44 h. In total 99 ml (410 mmol, 7.8 eq) N,N-dimethylformamide di-tert-butylacetal is added in 12 portions over time. After cooling to rt the mixture is diluted with EtOAc, washed with aq. bicarbonate and brine, and dried over sodium sulfate. The residue is purified by chromatography on silica (flashmaster, hexane to hexane/EtOAc 95/5) to give the product in the form of a white solid.

MS (LC/MS): 188=[M+H-tBu]⁺

¹H-NMR (300 MHz, CDCl₃): 7.37 (s, 1H), 7.16 (s, 1H), 3.97 (s, 3H), 1.59 (s, 9H).

### b) 2-(Allyl-methyl-amino)-6-methoxy-isonicotinic acid tert-butyl ester

To a solution of 1.66 g (17.2 mmol, 1.4 eq) sodium tert-butylate in 40 ml dioxane are added 3.00 g (12.3 mmol) 2-chloro-6-methoxy-isonicotinic acid tert-butyl ester and 1.41 ml (14.8 mmol, 1.2 eq) N-allylmethylamine. The mixture is heated to 80° C. and 0.149 g (0.02 eq) of the catalyst SK-CC02-A are added as solution in 12 ml dioxane. The reaction is heated at 110° C. for 18 h. After cooling to rt the mixture is diluted with EtOAc, washed with aq. bicarbonate and brine, and dried over sodium sulfate. The solvents are evaporated at reduced pressure and the residue is purified by chromatography on silica (flashmaster, hexane to hexane/EtOAc 95/5) to give the product.

MS (LC/MS): 279=[M+H]⁺

¹H-NMR (300 MHz, CDCl₃): 6.63 (s, 1H), 6.51 (s, 1H), 5.91-5.78 (m, 1H), 5.15 (d, 2H), 4.17 (d, 2H), 3.89 (s, 3H), 3.05 (s, 3H), 1.57 (s, 9H).

### c) 2-(Allyl-methyl-amino)-6-methoxy-isonicotinic acid hydrochloride

A solution of 2.10 g (7.54 mmol) 2-(allyl-methyl-amino)-6-methoxy-isonicotinic acid tert-butyl ester in 4 N HCl in dioxane is stirred for 2.5 h. The solvent is evaporated and the residue crystallized from DCM/ether/hexane to give the product in the form of an off-white solid.

MS (LC/MS): 223=[M+H]⁺

¹H-NMR (400 MHz, CDCl₃): 6.70 (s, 1H), 6.63 (s, 1H), 5.94-5.84 (m, 1H), 5.21 (s, 1H), 5.18 (d, 1H), 4.23 (d, 2H), 3.94 (s, 3H), 3.10 (s, 3H).

### Building block A6: 2-But-3-enyl-6-methyl-isonicotinic acid hydrochloride

### a) 2-Chloro-6-methyl-isonicotinic acid tert-butyl ester

A suspension of 5.00 g (29.1 mmol) 2-chloro-6-methyl-isonicotinic acid in chloroform is heated to reflux and 14.0 ml (58.3 mmol, 2 eq) N,N-dimethylformamide-di-tert-butylacetal are added over a period of 30 min. After 1.5 h and 3.5 h another portion of each time 5 ml (20.8 mmol, 0.7 eq) N,N-dimethylformamide-di-tert-butylacetal is added. After 4.5 h the mixture is cooled to rt, diluted with EtOAc, washed with aq. bicarbonate and brine, dried over sodium sulfate and the solvents are evaporated at reduced pressure. The residue is purified by chromatography on silica (flashmaster, hexane to hexane/EtOAc 95/5) to give the product in the form of a white solid.

MS (LC/MS): 172=[M+H-tBu]⁺

¹H-NMR (400 MHz, CDCl₃): 7.65 (s, 1H), 7.60 (s, 1H), 2.63 (s, 3H), 1.63 (s, 9H).

### b) 2-But-3-enyl-6-methyl-isonicotinic acid tert-butyl ester

To a solution of 0.50 g (2.2 mmol) chloro-6-methyl-isonicotinic acid tert-butyl ester, 1.26 ml (13.2 mmol, 6 eq) 1-methyl-2-pyrrolidone and 39 mg (0.05 eq) iron(111)-acetylacetonate in 20 ml THF is added a solution of 2.6 ml (1M solution in THF, 2.6 mmol, 1.2 eq) 3-butenyl magnesium bromide in THF (prepared from magnesium turnings and 3-butenylbromide in THF). After 30 min at rt saturated aq. ammonium chloride solution is added slowly and the mixture is extracted with EtOAc. The organic layer is washed with aq. bicarbonate and brine, and dried over sodium sulfate. The solvent is evaporated at reduced pressure and the residue is purified by chromatography on silica (flashmaster, hexane to hexane/EtOAc 7/3) to give the product.

MS (LC/MS): 192=[M+H-tBu]⁺

¹H-NMR (400 MHz, CDCl₃): 7.49 (s, 1H), 7.48 (s, 1H), 5.96-5.86 (m, 1H), 5.10 (d, 1H), 5.02 (d, 1H), 2.94 (t, 2H), 2.62 (s, 3H), 2.53 (q, 2H), 1.63 (s, 9H).

### c) 2-But-3-enyl-6-methyl-isonicotinic acid hydrochloride

A solution of 355 mg (1.4 mmol) 2-but-3-enyl-6-methyl-isonicotinic acid tert-butyl ester in 12.6 ml (35 eq) 4 N HCl in dioxane is stirred at 60° C. (bath temperature) for 2 h. The solvent is evaporated at reduced pressure and the residue is washed with ether, filtered off and dried in vacuum to give the product.

MS (LC/MS): 192=[M+H]⁺

¹H-NMR (400 MHz, d6-DMSO): 8.02 (s, 1H), 8.00 (s, 1H), 5.92-5.82 (m, 1H), 5.09-5.01 (m, 2H), 3.18 (t, 2H), 2.79 (s, 3H), 2.56-2.53 (m, 2H).

### Building block A7: 3-Allyloxy-5-methoxy-benzoic acid

### a) 3-Allyloxy-5-methoxy-benzoic acid methyl ester

To a mixture of 200 mg (1.09 mmol, 1 eq) 3-hydroxy-5-methoxy-benzoic acid methyl ester and 600 mg (4.30 mmol, 3.95 eq) K₂CO₃ in 15 ml acetone is added 400 mg (3.27 mmol, 3 eq) allyl bromide and the reaction mixture is refluxed at 80° C. for 16 h. The mixture is diluted with 100 ml water and 200 ml DCM, the organic layer is separated and washed with brine, dried over sodium sulfate, filtered and concentrated to give the product.

¹H-NMR (400 MHz, CDCl₃): 7.19 (m, 2H), 6.65 (t, 1H), 6.15-5.95 (m, 1H), 5.44-5.25 (m, 2H), 4.58 (m, 2H), 3.90 (s, 3H), 3.81 (s, 3H).

### b) 3-Allyloxy-5-methoxy-benzoic acid

To a solution of 220 mg (0.98 mmol, 1.0 eq) 3-allyloxy-5-methoxy-benzoic acid methyl ester in 5 ml MeOH and 1.5 ml water are added 130 mg (3.07 mmol, 3.13 eq) LiOH*H₂O and the reaction mixture is stirred for 20 hours at rt. The mixture is diluted with 50 ml DCM and 1 N HCl is added until the pH<2. The combined organic solvents are separated and washed with brine, dried over sodium sulfate, filtered and concentrated to give the product.

Rf: (EtOAc/hexane=20/80): 0.02

¹H-NMR (400 MHz, CDCl₃): 7.24 (m, 2H), 6.75 (m, 1H), 6.15-6.00 (m, 1H), 5.50-5.30 (m, 2H), 4.60 (m, 2H), 3.83 (s, 3H).

### Building block A8: 3-(Allyl-benzyloxycarbonyl-amino)-5-trifluoromethyl-benzoic acid

### a) 3-Benzyloxycarbonylamino-5-trifluoromethyl-benzoic acid

To a solution of 1.00 g (4.73 mmol, 1 eq) 3-amino-5-trifluoromethyl-benzoic acid in 5 ml THF and 15 ml sat. aq. NaHCO₃ are added 1.58 ml (4.70 mmol, 1 eq) benzyl chloroformate and the reaction mixture is stirred for 18 h at rt. The mixture is diluted with 50 ml 2 N HCl and 100 ml EtOAc. The organic solvents are separated, washed with brine, dried over sodium sulfate, filtered and concentrated to give the product.

MS (ES−): 338=[M−H]⁻

¹H-NMR (400 MHz, d6-DMSO): 8.38 (s, 1H), 8.15 (s, 1H), 7.80 (s, 1H), 7.49-7.30 (m, 5H), 5.20 (s, 2H).

### b) 3-Benzyloxycarbonylamino-5-trifluoromethylbenzoic acid methyl ester

To a solution of 1.80 g (5.15 mmol, 1 eq) 3-benzyloxycarbonylamino-5-trifluoromethyl-benzoic acid in 15 ml MeOH are added at 0° C. 567 μl (7.72 mmol, 1.5 eq) thionyl chloride and the reaction is stirred for 72 h. The mixture is diluted with 50 ml ethyl acetate and 20 ml water. The organic solvents are separated, washed with aq. NaHCO₃ and brine, dried over sodium sulfate, filtered and concentrated. The residue is purified by column chromatography using EtOAc/hexane (1/1) to give the product.

MS (ES−): 352=[M−H]

¹H-NMR (400 MHz, CDCl₃): 8.15 (s, 1H), 8.10 (s, 1H), 8.02 (s, 1H), 7.50-7.37 (m, 5H), 6.92 (s, 1H), 5.26 (s, 2H), 3.98 (s, 3H).

### c) 3-(Allyl-benzyloxycarbonyl-amino)-5-trifluoromethyl-benzoic acid methyl ester

To a solution of 1.70 g (4.67 mmol, 1 eq) 3-benzyloxycarbonylamino-5-trifluoromethylbenzoic acid methyl ester in 40 ml THF are added slowly 205 mg (5.13 mmol, 1.1 eq) NaH (60% in mineral oil) and the reaction mixture is stirred for 30 min. 174 mg (0.467 mmol, 0.1 eq) Tetrabutylammonium iodide and 400 μl (4.71 mmol, 1.01 eq) allyl bromide are added. After 23 h, 100 mg (2.5 mmol) NaH (60% in mineral oil) and 100 μl (1.18 mmol) allyl bromide are added and the reaction mixture is stirred for 1 h. The reaction mixture is dissolved with 20 ml water and 70 ml DCM. The organic layer is separated, washed with brine, dried over sodium sulfate, filtered and concentrated. The residue is purified by column chromatography using EtOAc/hexane (311) to give the product.

HPLC [Nucleosil C-18HD, 4×70 mm, 3 μm, 1 ml/min, 20-100% AcCN (6 min)]: Rt=6.28 min

¹H-NMR (400 MHz, CDCl₃): 8.18 (s, 1H), 8.16 (s, 1H), 7.77 (s, 1H), 7.48-7.28 (m, 5H), 6.00-5.88 (m, 1H), 5.30-5.18 (m, 4H), 4.40 (d, 2H), 3.98 (s, 3H).

### d) 3-(Allyl-benzyloxycarbonyl-amino)-5-trifluoromethyl-benzoic acid

To a solution of 1.85 g (4.69 mmol, 1 eq) 3-(allyl-benzyloxycarbonyl-amino)-5-trifluoromethyl-benzoic acid methyl ester in 15 ml MeOH are added 15 ml 1 N aq LiOH and the reaction mixture is stirred for 16 h. The mixture is diluted with 100 ml DCM and 30 ml 1 N aq HCl. The organic layer is separated, washed with brine, dried over sodium sulfate, filtered and concentrated to give the product.

MS (ES−): 378=[M−H]⁻

¹H-NMR (400 MHz, CDCl₃): 8.21 (s, 2H), 7.82 (s, 1H), 7.48-7.28 (m, 5H), 6.01-5.90 (s, 1H), 5.30-5.18 (m, 4H), 4.40 (d, 2H).

### Building block A9: 3-(Allyl-tert-butoxycarbonyl-amino)-benzoic acid

### a) 3-tert-Butoxycarbonylamino-benzoic acid ethyl ester

To a solution of 20 g (118 mmol, 1 eq) 3-amino-benzoic acid ethyl ester in 350 ml THF is added a solution of 26.4 g (118 mmol, 1 eq) BOC-anhydride in 130 ml THF and the reaction mixture is stirred for 18 h at rt. The mixture is diluted with EtOAc and washed with aqueous sodium bicarbonate solution and brine. The solvent is evaporated at reduced pressure and the residue is purified by chromatography on silica (flashmaster, hexane/EtOAc 9/1 to 4/1) to give the product.

MS (LC/MS): 288=[M+H+Na]⁺

¹H-NMR (400 MHz, CDCl₃): 7.95 (s, 1H), 7.75 (d, 2H), 7.40 (t, 1H), 6.69 (s, 1H), 4.40 (q, 2H), 1.56 (s, 9H), 1.42 (t, 3H).

### b) 3-(Allyl-tert-butoxycarbonyl-amino)-benzoic acid ethyl ester

To the suspension of 11 g (41 mmol, 1 eq) 3-tert-butoxycarbonylamino-benzoic acid ethyl ester and 20.3 g (62 mmol, 1.5 eq) caesium carbonate in 300 ml DMF are added 4.4 ml (52 mmol, 1.25 eq) allylbromid and the reaction mixture is stirred at 55° C. (bath temperature). After 18 h another 4.0 ml (47 mmol, 1.1 eq) allylbromid are added and the reaction is stirred again 5 h at 55° C. (bath temperature). Following the addition of 3.5 ml (41 mmol, 1 eq) allylbromid and 7 g (21 mmol, 0.5 eq) caesium carbonate the reaction mixture is stirred 5 h at 55° C. (bath temperature) and 5 days at rt. The mixture is diluted with EtOAc and washed with brine. The organic layer is dried over sodium sulfate, the solvent is evaporated at reduced pressure and the residue is purified by chromatography on silica (flashmaster, hexane to hexane/EtOAc 9/1) to give the product.

MS (LC/MS): 328=[M+H+Na]⁺

¹H-NMR (400 MHz, CDCl₃): 7.94 (s, 1H), 7.89 (d, 1H), 7.46 (d, 1H), 7.41 (t, 1H), 5.99-5.89 (m, 1H), 5.20 (d, 1H), 5.17 (s, 1H), 4.40 (q, 2H), 4.29 (d, 2H), 1.48 (s, 9H), 1.42 (t, 3H).

### c) 3-(Allyl-tert-butoxycarbonyl-amino)-benzoic acid

To the solution of 3.0 g (9.8 mmol, 1 eq) 3-(allyl-tert-butoxycarbonyl-amino)-benzoic acid ethyl ester in 50 ml ethanol are added 10.8 ml (10.8 mmol, 1.1 eq) of 1 N aqueous lithium hydroxide solution. The reaction is stirred 3 h at 50° C. (bath temperature). Following the addition of another 10 ml (10 mmol, 1 eq) 1 N aqueous lithium hydroxide solution the reaction mixture is stirred over night at 50° C. (bath temperature). The reaction mixture is acidified by addition of 0.1 N hydrochloric acid and extracted with EtOAc. The organic layer is washed with brine and dried over sodium sulfate. The solvent is evaporated at reduced pressure to give the product.

MS (LC/MS): 300=[M+H+Na]⁺

¹H-NMR (400 MHz, CDCl₃): 8.02 (br s, 1H), 7.96 (d, 1H), 7.54 (d, 1H), 7.46 (t, 1H), 6.01-5.91 (m, 1H), 5.3-5.2 (m, 1H), 5.19 (br s, 1H), 4.31 (d, 2H), 1.50 (s, 9H).

### Building block A10: 2-But-3-enyl-6-methoxy-isonicotinic acid

The title compound is obtained by an analogous reaction sequence as for building block A6, starting from 2-chloro-6-methoxy-isonicotinic acid tert-butyl ester (see building block A5) and 3-butenyl magnesium bromide.

MS (LC/MS): 208=[M+H]⁺

¹H-NMR (400 MHz, d6-DMSO): 7.28 (s, 1H), 7.02 (s, 1H), 5.93-5.82 (m, 1H), 5.07 (d, 1H), 4.98 (d, 1H), 3.90 (s, 3H), 2.85 (t, 2H), 2.47 (q, 2H).

### Building block A11: 3-(Allyl-tert-butoxycarbonyl-amino)-5-methoxy-benzoic acid

### a) 3-tert-Butoxycarbonylamino-5-methoxy-benzoic acid methyl ester

To the solution of 1.57 g (8.7 mmol, 1 eq) 3-amino-5-methoxy-benzoic acid methyl ester (CAS 217314-47-1, can be prepared according to literature procedures) and 0.11 g (0.87 mmol, 0.1 eq) 4-dimethylaminopyridine in 18 ml THF is added the solution of 1.89 g (8.7 mmol, 1 eq) BOC-anhydride in 7 ml THF. The reaction mixture is stirred at rt over night. After dilution with EtOAc the mixture is washed with aqueous sodium bicarbonate and brine, dried over sodium sulfate, and the solvents are evaporated at reduced pressure. The residue is purified by chromatography on silica (flashmaster, hexane to hexane/EtOAc 7/3) to give 0.83 g (2.9 mmol, 34%) product as white solid.

MS (LC/MS): 182=[M+H]⁺

¹H-NMR (400 MHz, CDCl₃): 7.54-7.51 (m, 1H), 7.39 (br s, 1H), 7.29-7.28 (m, 1H), 6.59 (br s, 1H), 3.93 (s, 3H), 3.87 (s, 3H), 1.56 (s, 9H).

### b) 3-(Allyl-tert-butoxycarbonyl-amino)-5-methoxy-benzoic acid methyl ester

To the suspension of 0.38 g (1.35 mmol, 1 eq)) 3-tert-butoxycarbonylamino-5-methoxy-benzoic acid methyl ester and 0.66 g (2.0 mmol, 1.5 eq) cesium carbonate in 10 ml DMF are added 0.14 ml (1.7 mmol, 1.25 eq) allylbromide. The reaction mixture is stirred 20 h at 50° C. After dilution with EtOAc the mixture is washed with brine, dried over sodium sulfate, and the solvents are evaporated at reduced pressure. The residue is purified by chromatography on silica (flashmaster, hexane to hexane/EtOAc 4/1) to give 0.34 g (1.0 mmol, 78%) product as colorless oil.

MS (LC/MS): 344=[M+H+Na]⁺

¹H-NMR (400 MHz, CDCl₃): 7.57 (br s, 1H), 7.42 (br s, 1H), 7.04 (br s, 1H), 5.99-5.89 (m, 1H), 5.21 (d, 1H), 5.17 (s, 1H), 4.27 (d, 2H), 3.94 (s, 3H), 3.87 (s, 3H), 1.49 (s, 9H).

### c) 3-(Allyl-tert-butoxycarbonyl-amino)-5-methoxy-benzoic acid

The solution of 0.37 g (1.1 mmol, 1 eq)) 3-(allyl-tert-butoxycarbonyl-amino)-5-methoxy-benzoic acid methyl ester and 1.3 ml (1.3 mmol, 1.1 eq) 1 N aqueous lithium hydroxide in 5 ml methanol is stirred 1 h at rt and 1.5 h at 50° C. The reaction mixture is diluted with EtOAc, washed with 0.1 N hydrochloric acid and brine, and dried over sodium sulfate. Evaporation of the solvents at reduced pressure gives 0.37 g (quantitative yield) product as yellowish oil.

MS (LC/MS): 330=[M+H+Na]⁺

¹H-NMR (400 MHz, CDCl₃): 7.60 (br s, 1H), 7.43 (br s, 1H), 7.07 (br s, 1H), 5.96-5.86 (m, 1H), 5.19 (d, 1H), 5.15 (s, 1H), 4.25 (d, 2H), 3.85 (s, 3H), 1.47 (s, 9H).

### Building block A12: 2-But-3-enyl-isonicotinic acid

### a) 2-Chloro-isonicotinic acid tert-butyl ester

A solution of 10.0 g (63.4 mmol) 2-chloro-isonicotinic acid in 100 ml chloroform is heated to reflux temperature. In total 91.2 ml (380 mmol, 6 eq) N,N-dimethylformamide di-tert-butylacetal is added in 3 portions of each 30.4 ml at the start, after 1 h and after 2 h. After cooling to rt the mixture is diluted with EtOAc, washed with aq. bicarbonate and brine, and dried over sodium sulfate. The residue is purified by chromatography on silica (flashmaster, hexane to hexane/EtOAc 95/5) to give 7.6 g (35.6 mmol, 56%) of the product as white solid.

MS (LC/MS): 158=[M+H-tBu]⁺

¹H-NMR (300 MHz, CDCl₃): 8.55 (d, 1H), 7.85 (s, 1H), 7.76 (d, 1H), 1.64 (s, 9H).

### b) 2-But-3-enyl-isonicotinic acid

The title compound is obtained by an analogous reaction sequence as for building block A6, starting from 2-chloro-isonicotinic acid tert-butyl ester and 3-butenyl magnesium bromide.

MS (LC/MS): 178=[M+H]³⁰

¹H-NMR (400 MHz, CDCl₃+one drop D3COH): 8.88 (d, 1H), 8.25 (d, 1H), 8.22 (s, 1H), 5.92-5.82 (m, 1H), 5.10 (s, 1H), 5.06 (br s, 1H), 3.45 (t, 2H), 2.72 (q, 2H).

### Building block A13: 3-Allyloxy-5-(2-oxo-pyrrolidin-1-yl)-benzoic acid

### a) 3-Bromo-5-nitro-benzoic acid

To the solution of 7.92 g (43 mmol) 3-amino-5-nitro-benzoic acid in 4 ml water in an ice bath 48.8 ml (434 mmol, 10 eq) of aqueous 48% HBr are added. A saturated aqueous solution of 4.05 g (59 mmol, 1.35 eq) sodium nitrite is added over 10 min. The obtained solution is added to the solution of 9.36 g (65 mmol, 1.5 eq) copper bromide in 48.8 ml (434 mmol, 10 eq) of aqueous 48% HBr at 70° C. The mixture is heated for 45 min at 70° C. After cooling to rt diethylether is added and the organic layer is washed with water until neutral pH is reached. Drying over sodium sulfate and evaporation of the solvent at reduced pressure gives 9.14 g (37 mmol, 86%) product as yellow solid.

MS (ES−): 245/247=[M+H]⁺

¹H-NMR (400 MHz, d6-DMSO): 8.65 (s, 1H), 8.57 (s, 1H), 8.44 (s, 1H).

### b) 3-Nitro-5-(2-oxo-pyrrolidin-1-yl)-benzoic acid

The mixture of 3.37 g (13.7 mmol) 3-bromo-5-nitro-benzoic acid, 1.74 g (27.4 mmol, 2 eq) copper powder, 3.18 ml (41 mmol, 3 eq) 2-pyrrolidinone and 1.89 g (13.7 mmol, 1 eq) potassium carbonate is stirred at 150° C. for 16 h. Another 10 ml 2-pyrrolidinone, 1.74 g (27.4 mmol, 2 eq) copper powder and 1.89 g (13.7 mmol, 1 eq) potassium carbonate are added and the mixture is again stirred vigorously for 5.5 h at 150° C. After cooling to rt the reaction is diluted with DCM and 5% aqueous potassium carbonate. Solids are filtered off and the aqueous layer is acidified with 10% aqueous potassium hydrogensulfate solution. Extraction with DCM, drying over sodium sulfate, and evaporation of the solvent at reduced pressure gives some product. On standing more product precipitates from the aqueous layer. This is filtered off and dried in vacuum. In total of 2.7 g (10.8 mmol, 79%) product are obtained.

MS (LC/MS): 273=[M+H+Na]⁺

¹H-NMR (400 MHz, d6-DMSO): 8.83 (s, 1H), 8.52 (s, 1H), 8.37 (s, 1H). 3.98 (t, 2H), 2.60 (t, 2H), 2.16-2.08 (m, 2H).

### c) 3-Amino-5-(2-oxo-pyrrolidin-1-yl)-benzoic acid

To the solution of 2.19 g (8.75 mmol) 3-nitro-5-(2-oxo-pyrrolidin-1-yl)-benzoic acid in 50 ml MeOH/THF 3/2 are added 0.2 g Pd/C (10% Engelhard 4505) and the reaction is stirred at rt under hydrogen (1 atm) for 18 h. After filtration through celite the solvent is evaporated at reduced pressure to give 1.84 g (8.4 mmol, 95%).

MS (ES−): 219=[M−H]⁺

¹H-NMR (400 MHz, d6-DMSO): 7.34 (s, 1H), 7.18 (s, 1H), 6.98 (s, 1H). 5.46 (br s, 2H), 3.79 (t, 2H), 2.49 (t, 2H), 2.10-1.99 (m, 2H).

### d) 3-Hydroxy-5-(2-oxo-pyrrolidin-1-yl)-benzoic acid

To the solution of 1.33 g (6.0 mmol) 3-amino-5-(2-oxo-pyrrolidin-1-yl)-benzoic acid in 15 ml water and 0.75 ml (13.3 mmol, 2.2 eq) concentrated sulfuric at 0° C. are added 0.56 g (8.2 mmol, 1.35 eq) sodium nitrite. After addition of 10 ml water the reaction is heated to 90° C. After cooling to rt the reaction is extracted with EtOAc, the organic layer is dried over sodium sulfate and the solvent is evaporated at reduced pressure to give 1.13 g (5.1 mmol, 84%) product.

MS (ES−): 220=[M−H]⁺

¹H-NMR (400 MHz, d6-DMSO): 9.86 (s, 1H), 7.63 (s, 1H), 7.45 (s, 1H), 7.13 (s, 1H), 3.83 (t, 2H), 2.51 (t, 2H), 2.10-2.02 (m, 2H).

### e) 3-Allyloxy-5-(2-oxo-pyrrolidin-1-yl)-benzoic acid

To the solution of 1.57 g (7.1 mmol) 3-hydroxy-5-(2-oxo-pyrrolidin-1-yl)-benzoic acid in 5 ml DMF are added 1.02 g (21.3 mmol, 3 eq) 50% sodium hydride in oil. After gas evolution stops 2.53 ml (28.4 mmol, 4 eq) allylbromide are added and the reaction mixture is stirred 4 days at 60° C. The reaction is diluted with water and extracted with EtOAc. The organic layer is dried over sodium sulfate and the solvent is evaporated at reduced pressure. The resulting ester is redisolved in 40 ml MeOH and 0.347 g (8.2 mmol, 1.2 eq) lithiumhydroxide monohydrate are added. The reaction is stirred at rt for 48 h. After evaporation of part of the solvent at reduced pressure the reaction is taken into water and washed with EtOAc. The aqueous layer is acidified with potassium hydrogensulfate and extracted with EtOAc. Drying over sodium sulfate and evaporation of the solvent at reduced pressure gives 1.56 g (6.0 mmol, 84%) product.

MS (LC/MS): 284=[M+H+Na]⁺

¹H-NMR (400 MHz, CDCl₃): 7.91 (s, 1H), 7.72 (s, 1H), 7.47 (s, 1H), 6.14-6.05 (m, 1H), 5.49 (d, 1H), 5.35 (d, 1H), 4.65 (d, 2H), 3.95 (t, 2H), 2.69 (t, 2H), 2.26-2.17 (m, 2H).

### Building block A14: 3-(Allyl-methyl-amino)-benzoic acid

### a) 3-(Allyl-methyl-amino)-benzoic acid ethyl ester

The solution of 1.27 g (4.2 mmol) 3-(allyl-tert-butoxycarbonyl-amino)-benzoic acid (building block A9) in 1N hydrochloric acid in dioxane is stirred 3 h at rt. The solvent is evaporated at reduced pressure and the residue is dried in high vacuum. The residue is taken into 30 ml of DMF and 2.99 g (9.2 mmol, 2.2 eq) cesium carbonate are added followed by 0.39 ml (4.2 mmol, 1 eq) methyl iodide. The resulting suspension is stirred at 55° C. over night. Another 0.4 ml (4.3 mmol, 1 eq) methyl iodide are added and stirring at 55° C. is continued for 4 h after which again 0.4 ml (4.3 mmol, 1 eq) of methyl iodide are added. The mixture is stirred for 4 hr at 55° C. followed by 5 days at rt. The reaction is diluted with EtOAc, and washed with brine. The organic layer is dried over sodium sulfate and the solvents are evaporated at reduced pressure. The residue is purified by chromatography on silica (flashmaster, hexan to hexane/EtOAc 9/1) to give 0.36 g (1.6 mmol, 39%) of product.

MS (LC/MS): 220=[M+H]⁺

¹H-NMR (400 MHz, CDCl₃): 7.44 (br s, 1H), 7.40 (d, 1H), 7.30 (t, 1H), 6.92 (br d, 1H), 5.93-5.83 (m, 1H), 5.22-5.17 (m, 2H), 4.40 (q, 2H), 4.00 (d, 2H), 3.02 (s, 3H), 1.42 (t, 3H).

### b) 3-(Allyl-methyl-amino)-benzoic acid

The turbid mixture of 0.35 g (1.6 mmol) 3-(allyl-methyl-amino)-benzoic acid ethyl ester and 1.76 ml (1.76 mmol, 1.1 eq) 1 N aqueous lithium hydroxide in 8 ml DMF is stirred at 50° C. (bath temperature) over night. The reaction mixture is acidified with 0.1 N hydrochloric acid and diluted with EtOAc. The organic layer is washed with brine, dried over sodium sulfate and the solvents are evaporated at reduced pressure to give 0.25 g (1.3 mmol, 81%) product as off-white solid.

MS (LC/MS): 192=[M+H]⁺

¹H-NMR (400 MHz, CDCl₃): 7.49-7.48 (m, 1H), 7.34 (t, 1H), 6.98 (br d, 1H), 5.94-5.84 (m, 1H), 5.25-5.18 (m, 2H), 4.02 (d, 2H), 3.04 (s, 3H).

### Building block A15: 3-Allyloxy-benzoic acid

### a) 3-Allyloxy-benzoic acid ethyl ester

To the suspension of 5 g (30 mmol, 1 eq) 3-hydroxy-benzoic acid ethyl ester and 8.32 g (60 mmol, 2 eq) of potassium carbonate in 25 ml acetone are added 2.8 ml (33 mmol, 1.1 eq) allylbromide and the mixture is stirred 22 h at rt followed by 1.5 h at 40° C. (bath temperature). Another 0.5 ml (6 mmol, 0.2 eq) allylbromide are added and the mixture is stirred for 4 h at 40° C. The solids are filtered off and the residue is washed with acetone. The filtrate is evaporated at reduced pressure and the residue is purified by chromatography on silica (flashmaster, hexane to hexane/EtOAc 4/1) to give 5.9 g (28.6 mmol, 95%) product.

MS (LC/MS): 179=[M−Et+H]⁺

¹H-NMR (400 MHz, CDCl₃): 7.64 (d, 1H), 7.57 (br s, 1H), 7.33 (t, 1H), 7.11 (br d, 1H), 6.11-6.02 (m, 1H), 5.44 (br d, 1H), 5.31 (br d, 1H), 4.59 (br d, 2H), 4.38 (q, 2H), 1.41 (t, 3H).

### b) 3-Allyloxy-benzoic acid

To the solution of 5.9 g (28.6 mmol, 1 eq) 3-allyloxy-benzoic acid ethyl ester in 170 ml ethanol are added 85 ml (85 mmol, 3 eq) of 1 N aqueous sodium hydroxide solution and the mixture is stirred 3 h at rt. Most of the organic solvent is evaporated at reduced pressure and the remaining solution is acidified with 1 N hydrochloric acid. The solution is stored in a refrigerator over night and the crystallized product is filtered off, washed briefly with water and dried in vacuum at 40° C. to give 4.81 g (27 mmol, 94%) of product as white crystals.

MS (ES−): 177=[M−H]⁺

¹H-NMR (400 MHz, CDCl₃): 7.72 (d, 1H), 7.64 (br s, 1H), 7.38 (t, 1H), 7.18 (br d, 1H), 6.12-6.02 (m, 1H), 5.45 (br d, 1H), 5.33 (br d, 1H), 4.62 (br d, 2H).

### Building block A16: 3-But-3-enyl-benzoic acid

### a) 3-Trifluoromethanesulfonyloxy-benzoic acid methyl ester

To the solution of 10 g (65.7 mmol, 1 eq) 3-hydroxy-benzoic acid methyl ester in 150 ml DCM at 0° C. are added 11.4 ml (99 mmol, 1.5 eq) 2,6-lutidine followed by 13.6 ml (82 mmol, 1.25 eq) trifluoromethanesulfonic acid anhydride. After stirring 2 h in an ice bath the mixture is hydrolyzed by addition of saturated ammonium chloride solution and extracted with EtOAc. The organic layer is washed with 0.1 N hydrochloric acid, brine, aqueous sodium bicarbonate, and brine again. After drying over sodium sulfate the solvent is evaporated at reduced pressure and the black residue purified by chromatography on silica (flashmaster, hexan/EtOAc 95/5) to give 16.2 g (57 mmol, 87%) of product as a yellowish oil.

MS (LC/MS): 285=[M+H]⁺

¹H-NMR (400 MHz, CDCl₃): 8.22 (d, 1H), 7.89 (s, 1H), 7.60 (t, 1H), 7.52 (br d, 1H), 4.00 (s, 3H).

### b) 3-But-3-enyl-benzoic acid methyl ester

To the solution of 10 g (35 mmol, 1 eq) 3-trifluoromethanesulfonyloxy-benzoic acid methyl ester in 250 ml THF are added 0.62 g (1.8 mmol, 0.05 eq) iron(III) acetylacetonate, 20 ml (211 mmol, 6 eq) 1-methyl-2-pyrrolidon, followed by 42 ml (42 mmol, 1.2 eq) of a 1 N solution of 3-butenylmagnesium bromide in diethyl ether (the grignard reagent is prepared by standard procedure from magnesium turnings and 4-bromo-but-1-ene in diethylether). The reaction mixture is stirred at rt for 1 h and another 42 ml (42 mmol, 1.2 eq) if the grignard reagent is added. After 2.5 h the reaction mixture is hydrolyzed by addition of saturated ammonium chloride solution and extracted with EtOAc. The organic layer is washed with 0.1 N hydrochloric acid, brine, aqueous sodium bicarbonate, and brine again. After drying over sodium sulfate the solvent is evaporated at reduced pressure and the residue is purified by chromatography on silica (flashmaster, hexane to hexan/EtOAc 95/5) to give 2.14 g (11 mmol, 32%) of the product as a yellowish oil.

MS (LC/MS): 191=[M+H]⁺

¹H-NMR (400 MHz, CDCl₃): 7.92-7.89 (m, 2H), 7.44-7.37 (m, 2H), 5.93-5.83 (m, 1H), 5.07 (d, 1H), 5.02 (d, 1H), 3.96 (s, 3H), 2.80 (t, 2H), 2.43 (q, 2H).

### c) 3-But-3-enyl-benzoic acid

To the solution of 2.1 g (11 mmol, 1 eq) 3-but-3-enyl-benzoic acid methyl ester in 30 ml of methanol are added 12.4 ml (12.4 mmol, 1.1 eq) of 1 N aqueous lithium hydroxide. The mixture is stirred 1 h at rt followed by 3 h at 50° C. The reaction is extracted with EtOAc. The organic layer is washed with 0.5 N hydrochloric acid and brine, dried over sodium sulfate, and the solvent is evaporated at reduced pressure. The residue is purified by chromatography on silica (flashmaster, hexane to hexan/EtOAc 4/1) to give 1.56 g (8.9 mmol, 79%) product as white solid.

MS (LC/MS): 177=[M+H]⁺

¹H-NMR (400 MHz, CDCl₃): 8.00 (br s, 2H), 7.49-7.41 (m, 2H), 5.94-5.84 (m, 1H), 5.08 (d, 1H), 5.04 (d, 1H), 2.83 (t, 2H), 2.45 (q, 2H).

### Building block A17: 3-(Allyl-benzyloxycarbonyl-amino)-5-oxazol-2-yl-benzoic acid

### a) 3-Nitro-5-oxazol-2-yl-benzoic acid methyl ester

Mono-methyl-5-nitroisophtalate (20 g, 87.9 mmol, 1 eq) is suspended in toluene (300 ml). DMF (300 μl) and SOCl₂ (12.93 ml, 175.9 mmol, 2 eq) are added and the reaction mixture is stirred at 80° C. for 7 hours. The reaction mixture is concentrated to give white crystals. The crystals are dissolved in sulfolan (200 ml), then triazol (13.4 g, 194 mmol, 2.2 eq) is added, followed by K₂CO₃ (12.3 g, 88.0 mmol, 1 eq). The reaction mixture is stirred at 90° C. for 16 hours. The reaction mixture is then filtered and diluted with diethyl ether and 0.1N aq HCl solution. The organic layer is washed with water, dried over Na₂SO₄, filtered and concentrated. The residue is purified by column chromatography using acetone and hexane in a ratio 1/6 to give the product (2.60 g, 10.4 mmol, 12%). Rf=0.28 in EtOAc/Hexane=1/4.

MS: 249 (M+H)

¹H-NMR (400 MHz, CDCl₃): 9.10 (s, 1H), 9.04 (s, 1H), 8.93 (s, 1H), 7.83 (s, 1H), 7.39 (s, 1H), 4.03 (s, 3H).

### b) 3-Nitro-5-oxazol-2-yl-benzoic acid

3-Nitro-5-oxazol-2-yl-benzoic acid methyl ester (2.50 g, 10.0 mmol, 1 eq) is dissolved in MeOH (130 ml), THF (50 ml) and H₂O (40 ml). LiOH*H₂O (3.25 g, 76.7 mmol, 7.69 eq) is added and the reaction mixture is stirred at room temperature over night. The reaction mixture is diluted with EtOAc and aq 1N HCl solution, the organic layer is washed with brine, dried over Na₂SO₄, filtered and concentrated to give the product (2.20 g, 9.30 mmol, 93%).

MS: 235 (M+H), 233 (M−H)

¹H-NMR (400 MHz, d6-DMSO): 8.83 (s, 1H), 8.80 (s, 1H), 8.70 (s, 1H), 8.40 (s, 1H), 7.58 (s, 1H).

### c) 3-Amino-5-oxazol-2-yl-benzoic acid

3-Nitro-5-oxazol-2-yl-benzoic acid (1 g, 4.23 mmol, 1 eq) is dissolved in a mixture of MeOH (50 ml) and THF (25 ml). Pd on charcoal is added (100 mg, Engelhard 4505) and the reaction is stirred for 4 hours at room temperature at 1 bar of hydrogen. The reaction mixture is filtered and concentrated to give the product (800 mg, 3.88 mmol, 92%).

MS: 205 (M+1), 203 (M−1)

¹H-NMR (400 MHz, d6-DMSO): 8.20 (s, 1H), 7.70 (s, 1H), 7.41 (s, 1H), 7.39 (s, 1H), 7.30 (s, 1H), 5.70 (bs, 2H).

### d) 3-Benzyloxycarbonylamino-5-oxazol-2-yl-benzoic acid

3-Amino-5-oxazol-2-yl-benzoic acid (800 mg, 3.38 mmol, 1 eq) is suspended in THF (50 ml). Carbobenzoxychloride (1.47 ml, 50%, 4.40 mmol, 1.3 eq) in toluene is added, followed by saturated aq. NaHCO₃. The reaction is stirred at room temperature for 20 hours. HCl (2N in H₂O) and EtOAc are added. The organic layer is washed with brine, dried over Na₂SO₄, filtered and concentrated. The residue is purified by column chromatography using EtOAc/hexane/AcOH in a ratio of 50/49/1 to give the product (800 mg, 2.34 mmol, 69%).

MS: 339 (M+1), 337 (M−1)

Rf=0.35 (EtOAc/hexane/AcOH)=50/49/1

### e) 3-Benzyloxycarbonylamino-5-oxazol-2-yl-benzoic acid methyl ester

To the solution of SOCl₂ (2.11 ml, 28.7 mmol, 7 eq) in MeOH (20 ml) and THF (10 ml) is added slowly at 0° C. the solution of 3-benzyloxycarbonylamino-5-oxazol-2-yl-benzoic acid (1.4 g, 4.10 mmol, 1 eq) in MeOH (10 ml). The reaction mixture is stirred for 20 hours and then diluted with EtOAc and aq NaHCO₃. The organic layer is dried over Na₂SO₄, filtered and concentrated to give the product (1.4 g, 3.93 mmol, 96%).

MS: 353 (M+1), 351 (M−1)

Rf: 0.42 (EtOAc/hexane=1/1)

### f) 3-(Allyl-benzyloxycarbonyl-amino)-5-oxazol-2-yl-benzoic acid methyl ester

3-Benzyloxycarbonylamino-5-oxazol-2-yl-benzoic acid methyl ester (330 mg, 0.927 mmol, I eq) is dissolved in THF (10 ml). NaH (48 mg, 60%, 1.21 mmol, 1.3 eq) is added in portions, and the reaction mixture is stirred for 30 min at room temperature. TBAI (35 mg, 92.7 μmol, 0.1 eq) and allyl bromide (119 μl, 1.39 mmol, 1.5 eq) are added and the reaction mixture is stirred for 20 hours. The reaction is quenched with HCl (1N in H₂O) and the aqueous phase is extracted with EtOAc. The organic layer is washed with brine, dried over Na₂SO₄, filtered and concentrated to give the product (300 mg, 757 μmol, 82%).

MS: 393 (M+1)

¹H-NMR (400 MHz, CDCl₃): 8.60-8.55 (m, 1H), 8.17 (s, 1H), 8.00 (s, 1H), 7.72 (s, 1H), 7.32-7.24 (m, 6H), 6.0-5.8 (m, 1H), 5.20 (s, 3H), 5.18-5.15 (m, 1H), 4.38 (d, 2H), 3.95 (s, 3H).

### g) 3-(Allyl-benzyloxycarbonyl-amino)-5-oxazol-2-yl-benzoic acid

3-(Allyl-benzyloxycarbonyl-amino)-5-oxazol-2-yl-benzoic acid methyl ester (300 mg, 757 μmol, 1 eq) is dissolved in methanol (10 ml) and H₂O (4 ml). LiOH*H₂O (100 mg, 2.37 mmol, 3.13 eq) is added to the reaction mixture and the reaction is stirred for 8 hours at room temperature. The reaction mixture is diluted with HCl (1N in H₂O) and DCM, the combined organic solvents are separated and washed with brine, dried over Na₂SO₄, filtered and concentrated. The resulting crystals are washed with hexane and dried under vacuum to give the product (280 mg, 733 μmol, 97%).

MS: 379 (M+1), 377 (M−1)

¹H-NMR (400 MHz, d6-DMSO): 8.30 (m, 1H), 8.24 (m, 1H), 8.10 (dd, 1H), 7.95 (dd, 1H), 7.40 (s, 1H), 7.35-7.20 (m, 5H), 6.0-5.8 (m, 1H), 5.18-5.12 (m, 4H), 4.38 (d, 2H).

### Building block A18: 3-Acetoxy-5-allyloxy-benzoic acid

### a) 3-Allyloxy-5-hydroxy-benzoic acid methyl ester

3,5-Dihydroxy benzoic acid methyl ester (20 g, 118 mmol, 1 eq) is dissolved in DMF (80 ml) and cooled to −78° C., when NaH (5.60 g, 60%, 140 mmol, 1.19 eq) is added in portions. The reaction mixture is allowed to warm to 0° C. and then cooled to −25° C. Allyl bromide (15 ml, 170 mmol, 1.44 eq) is added and the reaction is warmed to room temperature. The reaction mixture is stirred at room temperature for 1 hour and then poured into NH₄Cl solution. The aqueous layer is extracted with diethyl ether, the organic layer is washed with brine, dried over Na₂SO₄, filtered and concentrated. The mixture is purified by column chromatography using EtOAc/Hexane in a ratio of 1/3 to give the product (8.10 g, 38.5 mmol, 33%).

MS: 209 (M+1), 207 (M−1)

¹H-NMR (400 MHz, CDCl₃): 7.22-7.17 (m, 2H), 6.65 (t, 1H), 6.15-6.00 (m, 1H), 5.50-5.30 (m, 2H), 4.60-4.54 (m, 2H), 3.90 (s, 3H).

### b) 3-Allyloxy-5-hydroxy-benzoic acid

3-Allyloxy-5-hydroxy-benzoic acid methyl ester (500 mg, 2.38 mmol, 1 eq) is dissolved in MeOH (8 ml) and LiOH (1N in H₂O, 8 ml, 8 mmol, 3.36 eq). The reaction mixture is stirred for 16 hours at room temperature. The reaction is then diluted with HCl (1N in H₂O) and EtOAc. The organic layer is washed with brine, dried over Na₂SO₄, filtered and concentrated to give the product (440 mg, 2.27 mmol, 95%).

MS: 193 (M+1)

HPLC (Nucleosil C18HD, 20-100% AcCN): retention time=3.09 min

### c) 3-Acetoxy-5-allyloxy-benzoic acid

3-Allyloxy-5-hydroxy-benzoic acid (400 mg, 2.04 mmol, 1 eq) is dissolved in pyridine (3 ml) and THF (4 ml). Acetyl chloride is added and the reaction mixture is stirred for 30 min. The reaction mixture is diluted with EtOAc and HCl (1N in H₂O). The organic layer is dried over Na₂SO₄, filtered and concentrated to give the product (420 mg, 1.78 mmol, 87%).

MS: 235 (M−1)

¹H-NMR (400 MHz, CDCl₃): 7.65 (s, 1H), 7.56 (s, 1H), 6.96-6.94 (m, 1H), 6.14-6.00 (m, 1H), 5.50-5.30 (m, 2H), 4.60 (d, 2H), 2.35 (s, 3H).

### Building block A19: 3-(Allyl-benzyloxycarbonyl-amino)-5-nitro-benzoic acid

The title compound is obtained by an analogous hydrolysis reaction as described as last step for building block A17, starting from 3-(allyl-benzyloxycarbonyl-amino)-5-nitro-benzoic acid methyl ester (building block A26).

MS: 355.0 (M−1)

¹H-NMR (400 MHz, CDCl₃): 8.80 (s, 1H), 8.48 (s, 1H), 8.40 (s, 1H), 7.42-7.30 (m, 5H), 6.00-5.90 (m, 1H), 5.30-5.10 (m, 4H), 4.42 (d, 2H).

### Building block A20: 3-Acetyl-methyl-amino-5-(allyl-benzyloxycarbonyl-amino)-benzoic acid

### a) 3-Acetylamino-5-(allyl-benzyloxycarbonyl-amino)-benzoic acid methyl ester

3-(Allyl-benzyloxycarbonyl-amino)-5-nitro-benzoic acid methyl ester (building block A26) (138 mg, 369 μmol, 1 eq) and SnCl2*H₂O (583 mg, 2.58 mmol, 7 eq) are dissolved in EtOH (10 ml). The reaction mixture is stirred at 70° C. for 3 hours. The reaction mixture is concentrated and suspended in pyridine (3 ml) and DCM (4 ml). Acetyl chloride (200 μl, 2.82 mmol, 7.63 eq) is added to the reaction mixture at 0° C. and the reaction mixture is stirred for 1 hour at room temperature.

The mixture is diluted with EtOAc and H₂O. The organic layer is washed with HCl (1 N in H₂O), followed by aq NaHCO₃. The organic layer is dried over Na₂SO₄, filtered and concentrated. The residue is purified by column chromatography using EtOAc/hexane in a ratio of 1/2 to give the product (50 mg, 129 μmol, 35%).

MS: 381 (M−1)

¹H-NMR (400 MHz, CDCl₃): 8.44 (s, 1H), 8.00 (s, 1H), 7.70 (s, 1H), 7.40-7.30 (m, 5H), 5.98-5.82 (m, 1H), 5.25-5.10 (m, 4H), 4.30 (d, 2H), 3.86 (s, 3H), 2.10 (s, 3H).

### b) 3-Acetyl-methyl-amino-5-(allyl-benzyloxycarbonyl-amino)-benzoic acid

3-Acetylamino-5-(allyl-benzyloxycarbonyl-amino)-benzoic acid methyl ester (40 mg, 104 μmol, 1 eq) is dissolved in THF (5 ml). NaH (15 mg, 60%, 370 μmol, 3.6 eq) is added, followed by MeI (30 μl, 476 μmol, 4.6 eq). The reaction mixture is stirred for 10 hours at room temperature and is then poured into 1N aq HCl solution. The aqueous layer is extracted with diethyl ether and the combined organic layers are dried over Na₂SO₄, filtered, and concentrated. The residue is dissolved in MeOH (5 ml) and 1N aq NaOH (5 ml), and the reaction mixture is stirred for 5 hours at room temperature. The reaction mixture is then poured into 1N aq HCl solution. The organic layer is diluted with diethyl ether, washed with aqueous NaHCO₃, dried over Na₂SO₄, filtered and concentrated. The residue is purified by column chromatography using EtOAc/Hexane/HCOOH in a ratio of 65/34/1 to give the product (29 mg, 75 μmol, 72%).

Rf: 0.30 (EtOAc/Hexane/HCOOH=65/34/1)

MS: 381 (M−1)

### Building block A21: 3-Allyloxy-5-(2-oxo-propoxy)-benzoic acid

### a) 3-Allyloxy-5-(2-oxo-propoxy)-benzoic acid methyl ester

3-Allyloxy-5-hydroxy-benzoic acid methyl ester (see building block A18a) (300 mg, 1.44 mmol, 1 eq) is dissolved in acetone (10 ml). KI (361 mg, 2.16 mmol, 1.5 eq), K₂CO₃ (603 mg, 4.32 mmol, 3 eq), and chloroacetone (192 μmol, 2.16 mmol, 1.5 eq) are added to the reaction mixture. The reaction is refluxed for 19 hours and then cooled to room temperature. HCl (1N in H₂O) and diethyl ether are added. The organic layer is washed with brine, dried over Na₂SO₄, filtered and concentrated. The residue is purified by column chromatography using EtOAc/hexane in a ratio of 1/4 to give the product (325 mg, 1.22 mmol, 85%).

HPLC (Nucleosil C18HD, 20-100% AcCN): retention time=4.55 min

MS: 265 (M+1), 263 (M−1).

¹H-NMR (400 MHz, CDCl3): 7.26 (t, 1H), 7.17 (t, 1H), 6.70 (t, 1H), 6.10-6.00 (m, 1H), 5.50-5.30 (m, 2H), 4.60-4.57 (m, 4H), 3.93 (s, 3H), 2.32 (s, 3H).

### b) 3-Allyloxy-5-(2-oxo-propoxy)-benzoic acid

3-Allyloxy-5-(2-oxo-propoxy)-benzoic acid is obtained by an analogous hydrolysis reaction as the last step for building block A17, starting from 3-allyloxy-5-(2-oxo-propoxy)-benzoic acid methyl ester.

MS: 249 (M−1)

HPLC (Nucleosil C18HD, 20-100% AcCN): retention time=3.57 min

### Building block A22: 3-Allyloxy-5-dimethylcarbamoyloxy-benzoic acid

### a) 3-Allyloxy-5-dimethylcarbamoyloxy-benzoic acid methyl ester

3-Allyloxy-5-hydroxy-benzoic acid methyl ester (see building block A18a) (1 g, 4.80 mmol, 1 eq) is dissolved in acetonitrile (16 ml) and stirred at 0° C. Dimethyl carbamoyl chloride (767 μl, 8.16 mmol, 1.7 eq) is added to the reaction mixture, followed by NaH (250 mg, 60%, 6.2 mmol, 1.3 eq). The reaction mixture is stirred for 3.5 hours at room temperature. The reaction mixture is concentrated and diluted with water and diethyl ether, and then basified with 1N aq NaOH. The organic layer is washed with brine, dried over Na₂SO₄, filtered and concentrated to give the product (1.34 g, 4.75 mmol, 98%).

HPLC (Nucleosil C18HD, 20-100% AcCN): retention time=4.82 min

¹H-NMR (400 MHz, CDCl3): 7.46 (t, 1H), 7.42 (t, 1H), 6.95 (t, 1H), 6.12-6.00 (m, 1H), 5.48-5.40 (m, 2H), 4.60 (d, 2H), 3.90 (s, 3H), 3.13 (s, 3H), 3.02 (s, 3H).

### b) 3-Allyloxy-5-dimethylcarbamoyloxy-benzoic acid

3-Allyloxy-5-dimethylcarbamoyloxy-benzoic acid is obtained by an analogous hydrolysis reaction as the last step for building block A17, starting from 3-allyloxy-5-dimethylcarbamoyloxy-benzoic acid methyl ester.

HPLC (Nucleosil C18HD, 20-100% AcCN): retention time=3.83 min

MS: 264 (M−1).

### Building block A23: 3-Allyloxy-5-methoxymethoxy-benzoic acid

### a) 3-Allyloxy-5-methoxymethoxy-benzoic acid methyl ester

3-Allyloxy-5-hydroxy-benzoic acid methyl ester (see building block A18a) (1 g, 4.80 mmol, 1 eq) is dissolved in DMF (5 ml) at 0° C. NaH (231 mg, 60%, 5.76 mmol, 1.2 eq) is added to the reaction mixture, followed by MOMCl (553 μl, 7.20 mmol, 1.5 eq). After 2 hours, another 100 mg of NaH (2.5 mmol, 0.52 eq) and another 180 μl of MOMCl (2.34 mmol, 0.49 eq) are added. After stirring for 1 hour, the reaction mixture is diluted with HCl (1N in H₂O) and diethyl ether. The organic layer is washed with brine, dried over Na₂SO₄, filtered and concentrated. The residue is purified by column chromatography using EtOAc/Hexane=1/3 to give the product (700 mg, 2.75 mmol, 57%).

MS: 253 (M+1)

HPLC (Nucleosil C18HD, 20-100% AcCN): retention time=5.18 min

¹H-NMR (400 MHz, CDCl3): 7.35 (t, 1H), 7.25 (t, 1H), 6.82 (m, 1H), 6.12-6.00 (m, 1H), 5.50-5.30 (m, 2H), 5.20 (s, 2H), 4.60 (s, 2H), 3.90 (s, 3H), 3.50 (s, 3H).

### b) 3-Allyloxy-5-methoxymethoxy-benzoic acid

3-Allyloxy-5-methoxymethoxy-benzoic acid is obtained by an analogous hydrolysis reaction as the last step for building block A17, from 3-allyloxy-5-methoxymethoxy-benzoic acid methyl ester.

HPLC (Nucleosil C18HD, 20-100% AcCN): retention time=4.08 min

MS: 239 (M+1), 237 (M−1).

### Building block A24: 3-Allyloxy-5-methoxymethyl-benzoic acid

### a) 3-Allyloxy-5-hydroxymethyl-benzoic acid methyl ester

3-Allyloxy-5-hydroxymethyl-benzoic acid methyl ester is obtained as described by Fang et al., J. Am. Chem. Soc. 1998, 8543-8544.

### b) 3-Allyloxy-5-methoxymethyl-benzoic acid methyl ester

3-Allyloxy-5-hydroxymethyl-benzoic acid methyl ester (1 g, 4.45 mmol, 1 eq) is dissolved in DMF (10 ml). NaH (356 mg, 60%, 8.9 mmol, 2 eq) is added in portions. MeI (557 μl, 8.91 mmol, 2 eq) is added, and the reaction mixture is stirred for 2 hours at room temperature. The mixture is diluted with aq 1N HCl and diethyl ether. The organic layer is dried over Na₂SO₄, filtered and concentrated to give the product (1.04 g, 4.41 mmol, 99%).

HPLC (Nucleosil C18HD, 20-100% AcCN): retention time=4.98 min

¹H-NMR (400 MHz, CDCl₃): 7.63 (s, 1H), 7.53 (s, 1H), 7.18 (s, 1H), 6.15-6.02 (m, 1H), 5.50-5.30 (m, 2H), 4.60 (d, 2H), 4.50 (s, 2H), 3.93 (s, 3H), 3.41 (s, 3H).

### c) 3-Allyloxy-5-methoxymethyl-benzoic acid

3-Allyloxy-5-methoxymethyl-benzoic acid is obtained by an analogous hydrolysis reaction as the last step for building block A17, starting from 3-allyloxy-5-hydroxymethyl-benzoic acid methyl ester.

HPLC (Nucleosil C18HD, 20-100% AcCN): retention time=3.48 min

MS: 221 (M−1).

### Building block A25: 3-Allyloxy-5-dimethylcarbamoylmethoxy-benzoic acid 3-Allyloxy-5-dimethylcarbamoylmethoxy-benzoic acid

3-Allyloxy-5-hydroxy-benzoic acid methyl ester (see building block A18a) (1.04 g, 5 mmol, 1 eq) is dissolved in DMF (15 ml) at 0° C. NaH (400 mg, 60%, 10 mmol, 2 eq) is added in portions, followed by addition of 2-chloro-dimethylacetamide (0.78 ml, 7.5 mmol, 1.5 eq). The reaction is stirred for 22 hours at room temperature. The reaction mixture is cooled to 5° C. and diluted with MTBE and 1N aq HCl. The organic layer is washed with H₂O, dried over Na₂SO₄, filtered and concentrated. The residue is purified by column chromatography using toluene/EtOAc/HCOOH in a ratio of 30/20/1 to give the product (238 mg, 853 μmol, 17%).

MS: 280 (M+1), 278 (M−1).

¹H-NMR (400 MHz, d6-DMSO): 7.08 (s, 1H), 7.02 (s, 1H), 6.77 (s, 1H), 6.10-6.00 (m, 1H), 5.43-5.23 (m, 2H), 4.85 (s, 2H), 4.60 (d, 2H), 3.00 (s, 3H), 2.83 (s, 3H).

### Building block A26: 3-(Allyl-benzyloxycarbonyl-amino)-5-nitro-benzoic acid methyl ester

The title compound is obtained by an analogous reaction sequence as described for building block A17 (steps A17d to A17f), starting from 3-amino-5-nitro-benzoic acid.

HPLC (Nucleosil C18HD, 20-100% AcCN): retention time=4.96 min

¹H-NMR (400 MHz, CDCl₃): 8.50 (s, 1H), 8.45 (s, 1H), 8.30 (s, 1H), 7.40-7.30 (m, 5H), 6.00-5.90 (m, 1H), 5.20-5.14 (m, 4H), 4.42 (d, 2H), 3.94 (s, 3H).

### Building block A27: 2-(Acetyl-allyl-amino)-6-chloro-isonicotinic acid

### a) 2-Acetylamino-6-chloro-isonicotinic acid ethyl ester

A mixture of 14 g 2-amino-6-chloro-isonicotinic acid ethyl ester (Temple et al. J. Heterocycl. Chem. 1970, 7, 451) (70 mmol) in 150 ml acetic anhydride (large excess) and 150 ml pyridine (large excess) is stirred at 60° C. in the presence of 244 mg (2 mmol) DMAP for 16 h. The mixture is concentrated in vacuo, taken up in EtOAc and washed with 1N HCl, brine and 10% aq. Na₂CO₃. Yield 11.2 g yellowish crystals (EtOH)

Rf: (hexane/EtOAc=2/1): 0.46

LC (Nucleosil C-18HD, 4×70 mm, 3 μm, 40-100% AcCN (6 min), 100% AcCN (1.5 min)): 3.619 min

MS (ES) [M+H]⁺=243, 245

¹H-NMR (400 MHz, CDCl₃): 8.65 (s, 1H), 7.97 (br s, 2H), 7.65 (s, 1H), 4.45 (q, 2H), 2.26 (s, 3H), 1.44 (t, 3H).

### b) 2-(Acetyl-allyl-amino)-6-chloro-isonicotinic acid ethyl ester

A mixture of 6.0 g (25 mmol) 2-acetylamino-6-chloro-isonicotinic acid ethyl ester, 10.36 g (75 mmol) potassium carbonate and 4.23 ml (50 mmol) allyl bromide in 25 ml DMF is stirred at 60° C. for 24 h. The cooled mixture is diluted with water and TBME. The organic phase is washed with water, dried over Na₂SO₄ and evaporated. The product is purified by chromatography on silica gel (hexane/EtOAc 9/1) to yield 5.38 g of the title compound as a yellowish oil.

Rf: (hexane/EtOAc=2/1): 0.46

LC (Nucleosil C-18HD, 4×70 mm, 3 μm, 40-100% AcCN (6 min), 100% AcCN (1.5 min)): 3.619 min

MS (ES) [M+H]⁺=283, 285; [MNa]⁺=305, 307.

¹H-NMR (400 MHz, CDCl₃): 7.96 (br s, 1H), 7.74 (s, 1H), 5.98-5.88 (m, 1H), 5.22-5.17 (m, 2H), 4.59 (d, 2H), 4.45 (q, 2H), 2.26 (s, 3H), 1.44 (t, 3H).

### c) 2-(Acetyl-allyl-amino)-6-chloro-isonicotinic acid

A solution of 1.08 g 2-(acetyl-allyl-amino)-6-chloro-isonicotinic acid ethyl ester (3.82 mmol) in 15 ml MeOH is treated with 5.5 ml 1N NaOH (5.5 mmol) and stirred at 25° C. for 30 minutes. The reaction is quenched with 6 ml 1 N HCl and with EtOAc extracted. Crystallization from a small amount of EtOAc gives 662 mg yellow crystals.

Rf: (EtOAc/2% AcOH): 0.60

LC (Nucleosil C-18HD, 4×70 mm, 3 μm, 20-100% AcCN (6 min), 100% AcCN (1.5 min)): 3.475 min

MS (ES) [M+H]⁺=255, 257; [MNa]⁺=277, 279

¹H-NMR (400 MHz, CDCl₃): 8.04 (br s, 1H), 7.79 (s, 1H), 6.00-5.92 (m, 1H), 5.26-5.19 (m, 2H), 4.60 (d, 2H), 2.32 (s, 3H).

### Building block A28: 3-Allyloxy-5-oxazol-2-yl-benzoic acid

### a) 5-Allyloxy-isophthalic acid dimethyl ester

To the solution of 50 g (233 mmol, 1 eq) 5-hydroxy-isophthalic acid dimethyl ester and 44.3 ml (513 mmol, 2.2 eq) allylbromide in 1000 ml acetone is added 74.1 g (536 mmol, 2.3 eq) potassium carbonate. The reaction mixture is stirred at reflux temperature over night (bath temperature 75° C.). The solid parts are filtered and washed with acetone. The filtrate is evaporated at reduced pressure to give the product as a white solid (61.1 g, quant.) that is used without further purification.

MS (LC/MS): 251=[M+H]⁺

¹H-NMR (400 MHz, CDCl₃): 8.31 (s, 1H), 7.80 (s, 2H), 6.13-6.04 (m, 1H), 5.47 (d, 1H), 5.36 (d, 1H), 4.66 (br d, 2H), 3.97 (s, 6H).

### b) 5-Allyloxy-isophthalic acid monomethyl ester

To the solution of 40 g (160 mmol, 1 eq) 5-allyloxy-isophthalic acid dimethyl ester in 640 ml methanol are added over 45 minutes 80 ml (160 mmol, 1.0 eq) of 2N aqueous sodium hydroxid. The reaction mixture is stirred another 3 h at rt and most of the methanol is evaporated at reduced pressure. The residue is acidified to pH 5 by addition of 56 ml 2N aqueous hydrochloric acid and the mixture is extracted with EtOAc. The organic layer is washed with water, dried over sodium sulfate and the solvent is evaporated at reduced pressure to give the product as white solid (28.7 g, 236 mmol, 76%).

MS (LC/MS): 237/259/281=[M+H/M+H+Na/M+H+2Na]⁺

¹H-NMR (400 MHz, CDCl₃): 8.40 (s, 1H), 7.87 (s, 2H), 6.15-6.06 (m, 1H), 5.50 (d, 1H), 5.38 (d, 1H), 4.69 (d, 2H), 3.99 (s, 3H).

### c) 5-Allyloxy-N-(2,2-dimethoxy-ethyl)-isophthalamic acid methyl ester

To the solution of 1 g (4.2 mmol, 1 eq) 5-allyloxy-isophthalic acid monomethyl ester in 100 ml DCM are added 0.39 ml (4.7 mmol, 1.1 eq) of oxalylchloride and 4 drops of DMF. The reaction mixture is stirred 1 h at rt. 50 ml 10% aqueous sodiumcarbonate is added followed by 0.50 ml (4.7 mmol, 1.1 eq) 2,2-dimethoxy-ethylamine. After stirring over night at rt the reaction mixture is extracted with EtOAc. The organic layer is washed with water and brine, dried over sodium sulfate and the solvents are evaporated at reduced pressure. The residue is purified by chromatography on silice (Flashmaster, DCM to CDM/methanol 95/5) to give the product as white solid (1.36 g, 4.2 mmol, 99%).

MS (LC/MS): 346=[M+H+Na]⁺

¹H-NMR (400 MHz, CDCl₃): 7.74 (br s, 1H), 7.65 (br s, 1H), 6.43 (br t, 1H), 6.12-6.04 (m, 1H), 5.47 (d, 1H), 5.36 (d, 1H), 4.66 (d, 2H), 4.53 (t, 1H), 3.97 (s, 3H), 3.65 (t, 2H), 3.48 (s, 6H).

### d) 5-Allyloxy-N-(2-oxo-ethyl)-isophthalamic acid methyl ester

The solution of 1.36 g (4.2 mmol) 5-allyloxy-N-(2,2-dimethoxy-ethyl)-isophthalamic acid methyl ester in 10 ml THF and 10 ml 2N aqueous hydrochloric acid is stirred at rt for 5 h. The reaction mixture is extracted with EtOAc. The organic layer is washed with water and brine, dried over sodium sulfate and the solvents are evaporated at reduce pressure to give the crude aldehyde that is used without further purification (1.16 g, 4.18 mmol, 99%).

MS (LC/MS): 278=[M+H]⁺

¹H-NMR (400 MHz, CDCl₃): 9.81 (s, 1H), 8.03 (s, 1H), 7.75 (br s, 1H), 7.66 (br s, 1H), 7.01 (br s, 1H), 6.13-6.03 (m, 1H), 5.47 (d, 1H), 5.35 (d, 1H), 4.66 (d, 2H), 4.47 (d, 2H), 3.97 (s, 3H).

### e) 3-Allyloxy-5-oxazol-2-yl-benzoic acid methyl ester

To the solution of 2.09 g (8.4 mmol, 2 eq) hexachloroethane and 2.22 g (8.4 mmol, 2 eq) triphenylphosphine in 40 ml acetonitrile is added the solution of 1.16 g (4.2 mmol, 1 eq) 5-allyloxy-N-(2-oxo-ethyl)-isophthalamic acid methyl ester in 20 ml acetonitrile. After stirring for 15 min 1.35 ml (16.8 mmol, 4 eq) pyridine are added and the reaction mixture is stirred at rt over night. The reaction is diluted with 200 ml of brine and extracted with EtOAc. The organic layer is washed with brine, dried over sodium sulfate and the solvents are evaporated. The residue is purified by chromatography on silica (Flashmaster, DCM to DCM/methanol 97/3) to give the product (0.74 g, 2.9 mmol, 68%).

MS (LC/MS): 260=[M+H]⁺

¹H-NMR (300 MHz, CDCl₃): 8.30 (s, 1H), 7.79 (br s, 1H), 7.73 (s, 1H), 7.66 (br s, 1H), 7.25 (s, 1H), 6.13-6.00 (m, 1H), 5.45 (d, 1H), 5.32 (d, 1H), 4.65 (d, 2H), 4.47 (d, 2H), 3.94 (s, 3H).

### f) 3-Allyloxy-5-oxazol-2-yl-benzoic acid

To the solution of 0.74 g (2.8 mmol) 3-allyloxy-5-oxazol-2-yl-benzoic acid methyl ester in 15 ml dioxane are added 3.1 ml (3.1 mmol, 1.1 eq) 1N aqueous lithium hydroxid. The reaction mixture is stirred for 3 h at bath temperature 50° C. Most of the solvent is evaporated at reduced pressure and the mixture is acidified to pH 3-4 by addition of 2N aqueous hydrochloric acid. The precipitate is filtered off, washed with water and dried in vacuum (0.61 g, 2.5 mmol, 87%).

MS (LC/MS): 268=[M+H]⁺

¹H-NMR (400 MHz, d6-DMSO): 8.29 (s, 1H), 8.13 (s, 1H), 7.72 (s, 1H), 7.59 (s, 1H), 7.45 (s, 1H), 6.14-6.04 (m, 1H), 5.45 (d, 1H), 5.32 (d, 1H), 4.76 (d, 2H).

### Building block A29: 5-Allyloxy-N,N-dimethyl-isophthalmic acid

### a) 5-Allyloxy-isophthalic acid monomethyl ester

5-Allyloxy-isophthalic acid monomethyl ester is obtained as described by Fang et al., J. Am. Chem. Soc. 1998, 8543-8544.

### b) 5-Allyloxy-N,N-dimethyl-isophthalmic acid methyl ester

A solution of 2.17 g (9.18 mmol) 5-allyloxy-isophthalic acid monomethyl ester in 9.2 ml thionylchloride is heated to reflux for 1 h. Excess thionylchloride is then removed under reduced pressure to yield 2.36 g of 3-allyloxy-5-chlorocarbonyl-benzoic acid methylester as a colorless oil which is used for the next step without further purification.

To a solution of 2.36 g (9.18 mmol) 3-allyloxy-5-chlorocarbonyl-benzoic acid methylester in 9 ml DCM are added at 0° C. 27.6 ml of a 1 M solution of dimethylamine in THF (3 eq.), after stirring the mixture at rt for 2 h 100 ml of a half-saturated aqueous ammonium chloride solution are added. The mixture is extracted with TBME (2×75 ml), the combined organic layers are washed with 50 ml water, dried over sodium sulfate and evaporated. The residue is purified by chromatography on silica gel (EtOAc) and gives 2.1 g of the desired product as colorless oil.

Rf: (EtOAc): 0.48

MS (ES+): 364=[M+H]⁺

¹H-NMR (400 MHz, d6-DMSO): 7.49-7.46 (m, 2H), 7.24-7.20 (m, 1H), 6.08-5.97 (m, 1H), 5.40 (dd, 1H), 5.27 (dd, 1H), 4.68 (d, 1H), 3.85 (s, 3H), 2.99 (br s, 3H), 2.88 (br s, 3H).

### c) 5-Allyloxy-N,N-dimethyl-isophthalamic acid

To a solution of 2 g (7.6 mmol) 5-allyloxy-N,N-dimethyl-isophthalamic acid methyl ester in 16.8 ml THF/MeOH (1:1) are added at 0° C. 8.4 ml 1 M KOH (1.1 eq.) and the mixture is stirred at rt for 3 h. The organic solvents are removed under reduced pressure, the aqueous phase is acidified with HCl to pH 2 and extracted with DCM/EtOH (80:20) (2×38 ml). The combined organic layers are washed with 8 ml water, dried over sodium sulfate and evaporated to give 1.96 g of the desired product as colorless solid.

m.p.: 93-95° C.

MS (ES+): 250=[M+H]⁺

¹H-NMR (400 MHz, d6-DMSO): 13.28 (br s, 1H), 7.48-7.43 (m, 2H), 7.18-7.13 (m, 1H), 6.09-5.98 (m, 1H), 5.39 (dd, 1H), 5.26 (dd, 1H), 4.64 (d, 1H), 2.97 (br s, 3H), 2.88 (br s, 3H).

### Building block A30: 3-Allyloxy-5-ethoxymethyl-benzoic acid

3-Allyloxy-5-hydroxymethyl-benzoic acid methyl ester (Fang et al., J. Am. Chem. Soc. 1998, 8543-8544) (3 g, 13.5 mmol, 1 eq) is dissolved in DMF (30 ml). NaH (1.08 g, 60%, 27 mmol, 2 eq) is added at 0° C., followed by KI (4.5 g, 27 mmol, 2 eq). After stirring for 10 min, ethyl bromide (2 ml, 27 mmol, 2 eq) is added. After 2 hours, NaH (1.08 g, 60%, 27 mmol, 2 eq) and ethyl bromide (2 ml, 27 mmol, 2 eq) are added and the reaction is stirred for another hour. The reaction mixture is poured into 1N HCl and diethyl ether. The organic layer is separated, dried over sodium sulfate, filtered and concentrated. The residue is purified by column chromatography using EtOAc/hexane/AcOH in a ratio of 30 to 60 to 1 to give the product (1.95 g, 8.3 mmol, 61%).

MS (ES−): 235=[M−H]⁻

HPLC (Nucleosil C18HD, 20-100% ACN): retention time=4.26 min

¹H-NMR (400 MHz, CDCl₃): 7.73 (s, 1H), 7.60 (s, 1H), 7.23 (s, 1H), 6.18-6.04 (m, 1H), 5.50-5.34 (m, 2H), 4.62 (d, 2H), 4.58 (s, 2H), 3.60 (q, 2H), 1.30 (t, 3H).

### Building block A31: 3-Allyloxy-5-(2,2,2-trifluoro-ethoxymethyl)-benzoic acid

### a) 3-Allyloxy-5-bromomethyl-benzoic acid methyl ester

3-Allyloxy-5-hydroxymethyl-benzoic acid methyl ester (Fang et al., J. Am. Chem. Soc. 1998, 8543-8544) (540 mg, 2.43 mmol, 1 eq) and triphenylphosphine (700 mg, 2.67 mmol, 1.1 eq) are dissolved in DCM at 0° C., followed by CBr₄ (886 mg, 2.67 mmol, 1.1 eq). The reaction is stirred for 2 hours at 0° C. and then for 2 hours at 20° C. The reaction mixture is concentrated and the residue purified by column chromatography using EtOAc/hexane in a ratio of 1 to 4 to give the product (630 mg, 2.21 mmol, 91%).

MS (MS+): 302=[M+NH₃]⁺

HPLC (Nucleosil C18HD, 65-100% ACN): retention time=2.21 min

¹H-NMR (400 MHz, CDCl₃): 7.70 (s, 1H), 7.55 (dd, 1H), 7.20 (dd, 1H), 6.18-6.04 (m, 1H), 5.50-5.35 (m, 2H), 4.62 (d, 2H), 4.50 (s, 2H), 3.94 (s, 3H).

### b) 3-Allyloxy-5-(2,2,2-trifluoro-ethoxymethyl)-benzoic acid methyl ester

Trifluoroethanol (477 μl, 6.63 mmol, 3 eq) is dissolved in DMF. NaH (133 mg, 60%, 3.32 mmol, 1.5 eq) is added at 0° C. and the reaction is stirred at 0° C. for 30 min. 3-Allyloxy-5-bromomethyl-benzoic acid methyl ester (630 mg, 2.21 mmol, 1 eq) is added and the reaction is stirred for 2 hours at room temperature. The reaction mixture is diluted with EtOAc and water, the organic layer is separated, dried over sodium sulfate, filtered and concentrated. The residue is purified by column chromatography using EtOAc/hexane in a ratio of 1 to 4 to give the product (526 mg, 1.72 mmol, 78%).

MS (ES+): 322=[M+NH₃]⁺

HPLC (Nucleosil C18HD, 20-100% ACN): retention time=5.66 min

¹H-NMR (400 MHz, CDCl₃): 7.63 (s, 1H), 7.58 (dd, 1H), 7.19 (dd, 1H), 6.18-6.04 (m, 1H), 5.50-5.35 (m, 2H), 4.72 (s, 2H), 3.95 (s, 3H), 3.88 (q, 2H).

### c) 3-Allyloxy-5-(2,2,2-trifluoro-ethoxymethyl)-benzoic acid

3-Allyloxy-5-(2,2,2-trifluoro-ethoxymethyl)-benzoic acid is obtained by an analogous hydrolysis reaction as for building block A17, starting from 3-allyloxy-5-(2,2,2-trifluoro-ethoxymethyl)-benzoic acid methyl ester.

MS (ES−): 289=[M−H]-HPLC (Nucleosil C18HD, 20-100% ACN): retention time=4.72 min

¹H-NMR (400 MHz, CDCl₃): 7.70 (s, 1H), 7.62 (dd, 1H), 7.22 (dd, 1H), 6.18-6.04 (m, 1H), 5.50-5.35 (m, 2H), 4.77 (s, 2H), 3.88 (q, 2H).

### Building block A32: 3-Allyloxy-5-methoxymethoxymethyl-benzoic acid

### a) 3-Allyloxy-5-methoxymethoxymethyl-benzoic acid methyl ester

3-Allyloxy-5-hydroxymethyl-benzoic acid methyl ester (Fang et al., J. Am. Chem. Soc. 1998, 8543-8544) (1 g, 4.5 mmol, 1 eq) is dissolved in DCM (15 ml), followed by di-isopropylethylamine (1.55 ml, 9.1 mmol, 2.02 eq). MOMCl (0.51 ml, 6.75 mmol, 1.5 eq) is added at 0° C., and the reaction is stirred for 60 hours at room temperature. The reaction mixture is diluted with EtOAc and water. The organic layer is dried over sodium sulfate, filtered and concentrated to give the product (1.19 g, 4.5 mmol, 99%).

HPLC (Nucleosil C18HD, 20-100% ACN): retention time=5.02 min

¹H-NMR (400 MHz, CDCl₃): 7.65 (s, 1H), 7.55 (s, 1H), 7.19 (s, 1H), 6.17-6.03 (m, 1H), 5.50-5.33 (m, 2H), 4.80 (s, 2H), 4.63-4.60 (m, 4H), 3.95 (s, 3H), 3.42 (s, 3H).

### b) 3-Allyloxy-5-methoxymethoxymethyl-benzoic acid

3-Allyloxy-5-methoxymethoxymethyl-benzoic acid is obtained by an analogous hydrolysis reaction as for building block A17, starting from 3-allyloxy-5-methoxymethoxymethyl-benzoic acid methyl ester.

HPLC (Nucleosil C18HD, 20-100% ACN): retention time=3.94 min

¹H-NMR (400 MHz, CDCl₃): 7.75 (s, 1H), 7.60 (s, 1H), 7.23 (s, 1H), 6.17-6.03 (m, 1H), 5.50-5.34 (m, 2H), 4.80 (s, 2H), 4.65-4.60 (m, 4H), 3.44 (s, 3H).

### Building block A33: 3-(Allyl-benzyloxycarbonyl-amino)-5-methoxymethyl-benzoic acid

### a) 3-Hydroxymethyl-5-nitro-benzoic acid methyl ester

Monomethyl-5-nitroisophtalate (22.5 g, 100 mmol, 1 eq) and Et₃N (16.7 ml, 120 mmol, 1.2 eq) are dissolved in THF (200 ml) and stirred at 0° C. Isopropylchloroformate (140 ml, 1 N in toluene, 140 mmol, 1.4 eq) is added within 30 min. After stirring for 90 min at 0° C., the reaction mixture is poured on ice and 50 ml of 0.1 N aqueous HCl, and then diluted with TBME. The organic layer is separated, dried over sodium sulfate, filtered and concentrated. The crude product is dissolved in 300 ml THF and stirred at room temperature. A solution of NaBH₄ (12.5 g, 330 mmol, 3.3 eq) in 100 ml of ice water is added within 15 min. After the reaction is stirred for 1 hour at room temperature, the mixture is diluted with TBME and water. The organic layer is washed with brine, dried over sodium sulfate, filtered and concentrated to give the product (12.9 g, 61 mmol, 61%).

HPLC (Nucleosil C18HD, 20-100% ACN): retention time=3.20 min

¹H-NMR (400 MHz, CDCl₃): 8.80 (s, 1H), 8.48 (s, 1H), 8.39 (s, 1H), 4.93 (s, 2H), 4.01 (s, 3H).

### b) 3-Methoxymethyl-5-nitro-benzoic acid methyl ester

3-Hydroxymethyl-5-nitro-benzoic acid methyl ester (8.0 g, 37.9 mmol, 1 eq) is dissolved in 80 ml of DMF. NaH (2.15 g, 49.3 mmol, 1.3 eq) is added at 0° C. The suspension is stirred for 30 min at room temperature, then methyliodide (4.57 ml, 49.3 mmol, 1.3 eq) is added. The reaction is stirred for 3 h at room temperature and is then quenched by the addition of 1N HCl and TBME. The organic layer is dried over sodium sulfate, filtered and concentrated. The residue is purified by column chromatography using EtOAc/hexane in a ratio of 1 to 3 to give the product (4.05 g, 17.8 mmol, 47%).

HPLC (Nucleosil C18HD, 20-100% ACN): retention time=4.45 min

¹H-NMR (400 MHz, CDCl₃): 8.80 (s, 1H), 8.43 (s, 1H), 8.38 (s, 1H), 4.61 (s, 2H), 4.00 (s, 3H), 3.52 (s, 3H).

### c) 3-Benzyloxycarbonylamino-5-methoxymethyl-benzoic acid methyl ester

3-Methoxymethyl-5-nitro-benzoic acid methyl ester (3.80 g, 16.9 mmol, 1 eq) is dissolved in EtOH (80 ml). SnCl₂*2H₂O (1.58 g, 7 mmol, 7 eq) is added and the reaction is heated at 75° C. for 90 min. The reaction mixture is diluted with EtOAc and aqueous NaHCO₃, the organic layer is separated, dried over sodium sulfate, filtered and concentrated. The obtained residue is dissolved in THF, and carbobenzoxychloride (0.4 ml, 1.30 mmol, 1.2 eq) is added to the reaction mixture, followed by aqueous NaHCO₃. The reaction mixture is stirred for 1 hour at room temperature. The organic layer is diluted with EtOAc, separated, dried over sodium sulfate, filtered and concentrated. The residue is purified by column chromatography using EtOAc/hexane in a ratio of 1 to 4 to give the product (4.78 g, 14.5 mmol, 87%).

MS (ES−): 328=[M−H]⁻

HPLC (Nucleosil C18HD, 20-100% ACN): retention time=5.04 min

¹H-NMR (400 MHz, CDCl₃): 7.94 (s, 1H), 7.84-7.70 (m, 2H), 7.46-7.38 (m, 5H), 6.82 (s, 1H), 5.25 (s, 2H), 4.52 (s, 2H), 3.93 (s, 3H), 3.42 (s, 3H).

### d) 3-(Allyl-benzyloxycarbonyl-amino)-5-methoxymethyl-benzoic acid methyl ester

3-Benzyloxycarbonylamino-5-methoxymethyl-benzoic acid methyl ester (1.98 g, 6 mmol, 1 eq) is dissolved in 25 ml of DMF. NaH (327 mg, 55%, 7.5 mmol, 1.25 eq) is added to the reaction mixture, and the mixture is stirred for 40 min at 0° C. Allyl bromide (653 μl, 7.5 mmol, 1.25 eq) is added, and the reaction mixture is stirred for 30 min at room temperature. The mixture is then poured on ice water and extracted with EtOAc. The organic layer is separated, dried over sodium sulfate, filtered and concentrated. The residue is purified by column chromatography using EtOAc/hexane in a ratio of 1 to 4 to give the product (1.33 g, 3.6 mmol, 60%).

MS (ES+): 387=[M+NH₃]⁺

HPLC (Nucleosil C18HD, 20-100% ACN): retention time=5.55 min

¹H-NMR (400 MHz, CDCl₃): 7.92-7.88 (m, 2H), 7.48 (s, 1H), 7.40-7.30 (m, 5H), 6.00-5.87 (m, 1H), 5.20-5.17 (m, 4H), 4.50 (s, 2H), 4.34 (d, 2H), 3.94 (s, 3H), 3.40 (s, 3H).

### e) 3-(Allyl-benzyloxycarbonyl-amino)-5-methoxymethyl-benzoic acid

3-(Allyl-benzyloxycarbonyl-amino)-5-methoxymethyl-benzoic acid is obtained by an analogous hydrolysis reaction as for building block A17, starting from 3-(allyl-benzyloxycarbonyl-amino)-5-methoxymethyl-benzoic acid methyl ester.

MS (ES−): 354=[M−H]⁻

HPLC (Nucleosil C18HD, 20-100% ACN): retention time=4.64 min

¹H-NMR (400 MHz, CDCl₃): 7.94 (s, 2H), 7.55 (s, 1H), 7.40-7.20 (m, 5H), 6.00-5.88 (m, 1H), 5.22-5.18 (m, 4H), 4.53 (s, 2H), 4.37 (d, 2H), 3.40 (s, 3H).

### Building block A34: 3-Allyl-5-oxazol-5-yl-benzoic acid

### a) 3-Allyloxy-5-formyl-benzoic acid methyl ester

3-Allyloxy-5-hydroxymethyl-benzoic acid methyl ester (Fang et al., J. Am. Chem. Soc. 1998, 8543-8544) (1 g, 4.45 mmol, 1 eq) is dissolved in DCM (40 ml) and Dess-Martin reagent (2.34 g, 5.35 mmol, 1.2 eq) is added. The reaction is stirred for 1 hour at room temperature. The mixture is diluted with ether and water. The organic layer is washed with aq Na₂CO₃, dried over sodium sulfate, filtered and concentrated to give the product (971 mg, 4.41 mmol, 99%).

HPLC (Nucleosil C18HD, 20-100% ACN): retention time=4.72 min

¹H-NMR (400 MHz, CDCl₃): 10.0 (s, 1H), 8.17 (s, 1H), 7.86 (dd, 2H), 7.62 (dd, 1H), 7.40-7.20 (m, 5H), 6.16-6.02 (m, 1H), 5.50-5.32 (m, 2H), 4.72 (d, 2H), 3.99 (s, 3H).

### b) 3-Allyloxy-5-oxazol-5-yl-benzoic acid methyl ester

3-Allyloxy-5-formyl-benzoic acid methyl ester (950 mg, 4.27 mmol, 1 eq) is dissolved in MeOH. K₂CO₃ (835 mg, 5.98 mmol, 1.4 eq) is added, followed by tosylmethylisocyanate (851 mg, 4.27 mmol, 1 eq). The reaction is refluxed for 3 hours. The mixture is diluted with DCM and aqueous NaHCO₃, the organic layer is separated, dried over sodium sulfate, filtered and concentrated. The residue is purified by column chromatography using EtOAc/hexane in a ratio of 1 to 4 to give the product (1 g, 3.86 mmol, 90%).

MS (ES+): 260=[M+H]⁺

HPLC (Nucleosil C18HD, 20-100% ACN): retention time=4.78 min

¹H-NMR (400 MHz, CDCl₃): 7.98 (s, 1H), 7.96 (s, 1H), 6.15-6.05 (m, 1H), 5.53-5.36 (m, 2H), 4.66 (d, 2H), 3.99 (s, 3H).

### c) 3-Allyloxy-5-oxazol-5-yl-benzoic acid

3-Allyloxy-5-oxazol-5-yl-benzoic acid is obtained by an analogous hydrolysis reaction as for building block A17, starting from 3-allyloxy-5-oxazol-5-yl-benzoic acid methyl ester.

MS (ES−): 244=[M−H]⁻

HPLC (Nucleosil C18HD, 20-100% ACN): retention time=3.66 min

### Building block A35: 3-(Allyl-benzyloxycarbonyl-amino)-5-oxazol-5-yl-benzoic acid

### a) 5-Benzyloxycarbonylamino-isophtalic acid monomethylester

Monomethyl-5-nitroisophtalate (50 g, 220 mmol, 1 eq) is dissolved in a mixture of 650 ml of MeOH and 350 ml of THF. 3 g of Pd/C are added, and the reaction is hydrogenated over night at 1 bar of H₂. The reaction mixture is filtered and concentrated to give the crude amine, which is dissolved in a mixture of THF (200 ml) and aqueous NaHCO₃ (400 ml). carbobenzoxychloride (62 ml, 50% in toluene, 184 mmol, 0.9 eq) is added and the reaction is stirred for 1 hour. Again carbobenzoxychloride (31 ml, 50% in toluene, 92 mmol, 0.45 eq) is added, and the reaction is stirred over night. The white solid precipitate is washed with water and diethyl ether to give the product (57.3 g, 174 mmol, 79%).

MS (ES−): 328=[M−H]⁻

HPLC (Nucleosil C18HD, 20-100% ACN): retention time=4.36 min

¹H-NMR (400 MHz, d6-DMSO): 8.40 (s, 1H), 8.38 (s, 1H), 8.17 (s, 1H), 7.50-7.37 (m, 5H), 5.21 (s, 2H), 3.92 (s, 3H).

### b) 5-Benzyloxycarbonylamino-5-hydroxymethyl-benzoic acid methyl ester

5-Benzyloxycarbonylamino-isophtalic acid monomethylester (10 g, 30.1 mmol, 1 eq) and NEt3 (5 ml, 36.1 mmol, 1.2 eq) are suspended in a mixture of THF (200 ml) and N-methylpyrrolidone (200 ml). Isopropylchloroformate (42 ml, 1 N in toluene, 42 mmol, 1.4 eq) is added, and the reaction is stirred for 30 min at 0° C. The reaction mixture is then diluted with ether and water. The organic layer is washed with 0.1 N HCl and brine. NaBH4 (3.82 g, 101 mmol, 3.36 eq) is dissolved in H₂O (100 ml) and added to the reaction mixture. The reaction is stirred for 1 hour, then diethyl ether and H₂O are added. The organic layer is separated, washed with brine, dried over Na2SO4, filtered and concentrated. The residue is purified by column chromatography using EtOAc/hexane in a ratio of 1 to 2 to give the product (1.5 g, 4.71 mmol, 16%).

MS (ES−): 314=[M−H]⁻

¹H-NMR (400 MHz, d6-DMSO): 8.07 (s, 1H), 7.68 (s, 1H), 7.60 (s, 1H), 7.50-7.37 (m, 5H), 5.39 (t, 1H), 5.20 (s, 2H), 4.54 (d, 2H), 3.86 (s, 3H).

### c) 5-Benzyloxycarbonylamino-5-oxazol-5-yl-benzoic acid methyl ester

5-Benzyloxycarbonylamino-5-hydroxymethyl-benzoic acid methyl ester (1.00 g, 3.14 mmol, 1 eq) is suspended in DCM (80 ml) and EtOAc (20 ml). Dess Martin reagent (1.65 g, 3.77 mmol, 1.2 eq) is added, and the reaction is stirred for 1 hour at room temperature. The reaction mixture is diluted with 0.1 N HCl solution and EtOAc. The organic layer is washed with brine, dried over sodium sulfate, filtered, and concentrated. The solid residue is suspended in a mixture of MeOH (80 ml) and EtOAc (40 ml). K₂CO₃ (800 mg, 5.73 mmol, 1.83 eq) and toluenesulphonylmethyl isocyanide (TosMIC) (800 mg, 4.10 mmol, 1.3 eq) are added, and the reaction is stirred for 1 hour at room temperature. The reaction is quenched with 1 N HCl solution and diluted with EtOAc. The organic layer is washed with brine, dried over sodium sulfate, filtered and concentrated. The residue is purified by column chromatography using EtOAc/hexane in a ratio of 1 to 2 to give the product (450 mg, 1.26 mmol, 40%).

MS (ES−): 351=[M−H]⁻

HPLC (Nucleosil C18HD, 20-100% ACN): retention time=4.83 min

¹H-NMR (400 MHz, d6-DMSO): 8.53 (s, 1H), 8.16-8.09 (m, 2H), 7.93 (s, 1H), 7.80 (s, 1H), 7.53-7.18 (m, 5H), 5.22 (s, 2H), 3.91 (s, 3H).

### d) 3-(Allyl-benzyloxycarbonyl-amino)-5-oxazol-5-yl-benzoic acid

5-Benzyloxycarbonylamino-5-oxazol-5-yl-benzoic acid methyl ester (380 mg, 1.07 mmol, 1 eq) is dissolved in DMF (10 ml). NaH (56 mg, 60%, 1.39 mmol, 1.3 eq) is added, and the mixture is stirred for 5 min at room temperature. Allyl bromide (137 μl, 1.60 mmol, 1.5 eq) is added, and the reaction mixture is stirred for 2 hours at room temperature. The reaction mixture is poured into a mixture of 1 N HCl and diethyl ether. The organic layer is separated, washed with brine, dried over sodium sulfate, filtered, and concentrated. The residue is dissolved in MeOH (15 ml) and 1 N LiOH in water (4.28 ml, 4.28 mmol, 4 eq). After 12 h, the reaction mixture is acidified with 1 N HCl and diluted with EtOAc. The organic layer is washed with brine, dried over sodium sulfate, filtered and concentrated. The residue is purified by column chromatography, using EtOAc/hexane/AcOH in a ratio of 50 to 50 to 1.

MS (ES+): 379=[M+H]⁺

¹H-NMR (400 MHz, CDCl₃): 8.28 (s, 1H), 8.10 (s, 1H), 8.01 (s, 1H), 7.82 (s, 1H), 7.50 (s, 1H), 7.40-7.36 (m, 5H), 6.02-5.92 (m 1H), 5.27-5.00 (m, 4H), 4.42 (d, 2H).

### Building block A36: 3-(Allyl-benzyloxycarbonyl-amino)-5-(2-oxo-propoxy)-benzoic acid

### a) 3-Benzyloxycarbonylamino-5-hydroxy-benzoic acid

3-Amino-5-hydroxy-benzoic acid (50 g, 323 mmol, 1 eq) is suspended in a mixture of THF (150 ml) and aqueous Na₂CO₃ (300 ml). Benzyl chloroformate (55.7 ml, 323 mmol, 1 eq) is added to the reaction mixture, and the reaction is stirred for 20 h at room temperature. The mixture is diluted with Et₂O and 4N HCl solution. The organic layer is separated, dried over sodium sulfate, filtered and concentrated to give the product (80 g, 85%).

HPLC (Nucleosil C18HD, 4×70 mm, 3 μm, 20-100% AcCN (6 min), 100% AcCN (1.5 min)) retention time=3.63 min

¹H-NMR (400 MHz, d6-DMSO): 9.80 (s, 1H), 7.60 (s, 1H), 7.47-7.32 (m, 5H), 7.24 (s, 1H), 7.00 (s, 1H), 5.20 (s, 2H).

### b) 3-Benzyloxycarbonylamino-5-hydroxy-benzoic acid methyl ester

3-Benzyloxycarbonylamino-5-hydroxy-benzoic acid (60 g, 208 mmol, 1 eq) is dissolved in MeOH (400 ml). SOCl₂ (23 ml, 313 mmol, 1.5 eq) is added at 0° C. The reaction is stirred at room temperature over night. The product is obtained by filtering and washing with water (62 g, 99%).

HPLC (Nucleosil C18HD, 4×70 mm, 3 μm, 20-100% AcCN (6 min), 100% AcCN (1.5 min)) retention time=4.32 min

¹H-NMR (400 MHz, d6-DMSO): 9.90 (s, 1H), 7.60 (s, 1H), 7.47-7.32 (m, 5H), 7.23 (s, 1H), 7.00 (s, 1H), 5.20 (s, 2H), 3.82 (s, 3H).

### c) 3-Benzyloxycarbonylamino-5-(1-ethoxy-ethoxy)-benzoic acid methyl ester

3-Benzyloxycarbonylamino-5-hydroxy-benzoic acid methyl ester (3 g, 10 mmol, 1 eq) is suspended in ethylvinylether (30 ml) and dioxane (10 ml). 5 ml of 4 N HCl in dioxane are added, and the suspension is stirred at room temperature over night, while it turns into a clear solution. 5 ml of Et₃N are added, and the reaction is stirred for 5 min. The reaction mixture is diluted with aqueous sodium bicarbonate, the organic layer is separated, dried over sodium sulfate, filtered and concentrated to give the product (3.64 g, 98%).

HPLC (Nucleosil C18HD, 4×70 mm, 3 μm, 20-100% AcCN (6 min), 100% AcCN (1.5 min)) retention time=4.32 min

¹H-NMR (400 MHz, d6-DMSO): 10.0 (s, 1H), 7.80 (s, 1H), 7.48-7.35 (m, 6H), 7.21 (s, 1H), 5.47 (q, 1H), 5.20 (s, 2H), 3.85 (s, 3H), 3-73-3.63 (m, 1H), 3.58-3.49 (m, 1H), 1.41 (d, 3H), 1.19-1-11 (m, 3H).

### d) 3-(Allyl-benzyloxycarbonyl-amino)-5-hydroxy-benzoic acid methyl ester

3-Benzyloxycarbonylamino-5-(1-ethoxy-ethoxy)-benzoic acid methyl ester (4.63 g, 12.4 mmol, 1 eq) is dissolved in 20 ml of DMF. Allyl bromide (1.6 ml, 18.6 mmol, 1.5 eq) is added, followed by NaH (300 mg, 6.2 mmol, 0.5 eq). After stirring for 30 min at 0° C., NaH (180 mg, 3.7 mmol, 0.3 eq) is added at 0° C., and the reaction is stirred at room temperature for 30 min. MeOH (3 ml) is added to the reaction mixture, and the reaction is stirred for 5 min. Then, 1 N aq. HCl (20 ml) is added, and the reaction mixture is stirred for 5 min. The reaction mixture is then diluted with Et₂O and water, the organic layer is separated, washed with brine, dried over sodium sulfate, filtered and concentrated. The residue is purified by column chromatography using EtOAc/hexane in a ratio of 1 to 2 to give the product (1.03 g, 24%).

MS (ES+): 359=[M+NH₄]⁺

HPLC (Nucleosil C18HD, 4×70 mm, 3 μm, 20-100% AcCN (6 min), 100% AcCN (1.5 min)) retention time=4.80 min

¹H-NMR (400 MHz, CDCl₃): 7.50 (s, 1H), 7.42-7.30 (m, 6H), 6.99 (s, 1H), 5.97-5.85 (m, 1H), 5.25-5.17 (m, 4H), 4.33 (d, 2H), 3.91 (s, 3H).

### e) 3-(Allyl-benzyloxycarbonyl-amino)-5-(2-oxo-propoxy)-benzoic acid methyl ester

3-(Allyl-benzyloxycarbonyl-amino)-5-hydroxy-benzoic acid methyl ester (1.03 g, 2.93 mmol, 1 eq) is dissolved in acetone (35 ml). Chloroacetone (0.4 ml, 4.4 mmol, 1.5 eq), potassium iodide (730 mg, 4.40 mmol, 1.5 eq), and K₂CO₃ (1.21 g, 3 eq) are added to the reaction mixture, and the reaction is refluxed over night. The mixture is filtered and concentrated. The residue is dissolved with Et₂O and 1 N aq. HCl. The organic layer is separated, dried over sodium sulfate, filtered and concentrated. The residue is purified by column chromatography using EtOAc/hexane in a ratio of 1 to 3 to give the product (800 mg, 69%).

MS (ES+): 415=[M+NH₄]⁺

HPLC (Nucleosil C18HD, 4×70 mm, 3 μm, 20-100% AcCN (6 min), 100% AcCN (1.5 min)) retention time=5.19 min

¹H-NMR (400 MHz, CDCl₃): 7.62 (s, 1H), 7.42-7.31 (m, 6H), 7.07 (s, 1H), 5.99-5.88 (m, 1H), 5.21-5.17 (m, 4H), 4.60 (s, 2H), 4.34 (d, 2H), 3.93 (s, 3H), 2.30 (s, 3H).

### f) 3-(Allyl-benzyloxycarbonyl-amino)-5-(2-oxo-propoxy)-benzoic acid

3-(Allyl-benzyloxycarbonyl-amino)-5-(2-oxo-propoxy)-benzoic acid can be obtained by an analogous hydrolysis reaction as for building block A17, starting from 3-(allyl-benzyloxycarbonyl-amino)-5-(2-oxo-propoxy)-benzoic acid methyl ester.

MS (ES+): 401=[M+NH₄]⁺

HPLC (Nucleosil C18HD, 4×70 mm, 3 μm, 20-100% AcCN (6 min), 100% AcCN (1.5 min)) retention time=4.41 min

### Building block A37: 2-Allyloxy-6-methyl-isonicotinic acid

To the solution of 2-chloro-6-methyl-isonicotinic acid (2.0 g, 11 mmol) in allylic alcohol (30 ml) is carefully added sodium hydride (0.59 g, 23 mmol, 2.1 eq) in small portions. After the addition the reactions is heated at reflux temperature under an atmosphere of nitrogen during 21 h. After cooling to rt another portion of sodium hydride (0.59 g, 23 mmol, 2.1 eq) is added and the mixture is heated at reflux over night. This procedure is repeated twice more. After cooling the mixture is poured on ice water and acidified to pH 3-4 by addition of 1 N aq. HCl. The mixture is extracted with EtOAc, the organic layers are dried over sodium sulfate and the solvent is evaporated at reduced pressure. The residue is purified by chromatography on silica gel (flashmaster, DCM to DCM/MeOH 95/5, with 0.5% AcOH) to give the product (1.4 g, 7.2 mmol, 65%).

MS (ES+): 194=[M+H]⁺

¹H-NMR (400 MHz, CD₃OD): 7.33 (s, 1H), 7.11 (s, 1H), 6.17-6.07 (m, 1H), 5.42 (d, 1H), 5.26 (d, 1H), 4.87 (d, 2H), 2.50 (s, 3H).

### Building block A38: 3-Allyloxy-5-(3-oxo-butyl)-benzoic acid

### a) 3-Allyloxy-5-bromomethyl-benzoic acid methyl ester

3-Allyloxy-5-hydroxymethyl-benzoic acid methyl ester (540 mg, 2.43 mmol, 1 eq) is dissolved in DCM (25 ml) and stirred at 0° C. Triphenylphosphin (700 mg, 2.67 mmol, 1.1 eq) is added to the reaction mixture, followed by CBr₄ (886 mg, 2.67 mmol, 1.1 eq). The reaction is stirred at 0° C. for 2 h, then for 2 h at room temperature. The reaction mixture is concentrated, and the residue is purified by column chromatography using EtOAc/hexane in a ratio of 1 to 4 to give the product (630 mg, 91%).

MS (ES+): 302 and 304=[M+NH₄]⁺

HPLC (Nucleosil C18HD, 4×70 mm, 3 μm, 65-100% AcCN (6 min), 100% AcCN (1.5 min)) retention time=2.11 min

¹H-NMR (400 MHz, CDCl₃): 7.70 (s, 1H), 7.55 (dd, 1H), 7.19 (dd, 1H), 6.15-6.02 (m, 1H), 5.50-5.32 (m, 2H), 4.62 (d, 2H), 4.50 (s, 2H), 3.96 (s, 3H).

### b) 3-Allyloxy-5-(2-methoxycarbonyl-3-oxo-butyl)-benzoic acid methyl ester

Sodium ethanolate (633 mg, 9.30 mmol, 1 eq) is suspended in EtOH (10 ml) and methyl acetoacetate (1.00 ml, 9.30 mmol, 1 eq) is added. After 30 min, 3-allyloxy-5-bromomethyl-benzoic acid methyl ester (2.65 g, 9.30 mmol, 1 eq) is added to the reaction mixture. The reaction is refluxed for 4 h and then cooled to room temperature. The mixture is diluted with EtOAc and brine. The organic layer is separated, dried over sodium sulfate, filtered and concentrated. The residue is purified by column chromatography using EtOAc/hexane in a ratio of 1 to 4 to give the product (700 mg, 23%).

MS (ES+): 338=[M+NH₄]⁺

HPLC (Nucleosil C18HD, 4×70 mm, 3 μm, 20-100% AcCN (6 min), 100% AcCN (1.5 min)) retention time=4.99 min

¹H-NMR (400 MHz, CDCl₃): 7.50 (s, 1H), 7.46 (s, 1H), 6.99 (s, 1H), 6.14-6.02 (m, 1H), 5.48-5.32 (m, 2H), 4.60 (d, 2H), 3.95 (s, 3H), 3.84 (t, 1H), 3.75 (s, 3H), 3.20 (d, 2H), 2.23 (s, 3H).

### c) 3-Allyloxy-5-(3-oxo-butyl)-benzoic acid

3-Allyloxy-5-(2-methoxycarbonyl-3-oxo-butyl)-benzoic acid methyl ester (700 mg, 2.18 mmol) is dissolved in EtOH (20 ml). After the addition of 4 N aq.NaOH (2.18 ml) and water (10 ml), the reaction is refluxed for 4 h. The mixture is diluted with 1 N HCl and EtOAc, the organic layer is separated, dried over sodium sulfate, filtered and concentrated. The residue is purified by column chromatography using EtOAc to give the product (288 mg, 53%).

MS (ES+): 266=[M+NH₄]⁺

HPLC (Nucleosil C18HD, 4×70 mm, 3 μm, 20-100% AcCN (6 min), 100% AcCN (1.5 min)) retention time=3.84 min

¹H-NMR (400 MHz, CDCl₃): 7.60 (s, 1H), 7.50 (s, 1H), 7.06 (s, 1H), 6.17-6.03 (m, 1H), 5.50-5.32 (m, 2H), 4.61 (d, 2H), 2.96 (t, 2H), 2.82 (t, 2H), 2.20 (s, 3H).

### Building block A39: 3-Acetyl-5-allyloxy-benzoic acid

### a) 3-Allyloxy-5-(1-hydroxy-ethyl)-benzoic acid methyl ester

3-Allyloxy-5-formyl-benzoic acid (800 mg, 3.63 mmol, 1 eq) is dissolved in THF (15 ml) and cooled to −78° C. Methylmagnesium chloride (1.85 ml, 22% in THF, 5.45 mmol, 1.5 eq) is added at −78° C. The reaction is stirred at −78° C. for 1 hour and then quenched by the addition of aqueous ammonium choloride. The reaction mixture is warmed to room temperature and diluted with water and EtOAc. The organic layer is separated, washed with brine, dried over sodium sulfate, filtered and concentrated. The residue is purified by column chromatography using EtOAc/hexane in a ratio of 1 to 9 to give the product (570 mg, 66%).

MS (ES+): 254=[M+NH₄]⁺

HPLC (Nucleosil C18HD, 4×70 mm, 3 μm, 20-100% AcCN (6 min), 100% AcCN (1.5 min)) retention time=4.07 min

¹H-NMR (400 MHz, CDCl₃): 7.66 (s, 1H), 7.50 (dd, 1H), 7.20 (dd, 1H), 6.17-6.02 (m, 1H), 5.50-5.33 (m, 2H), 4.94 (q, 1H), 4.62 (d, 2H), 3.94 (s, 3H), 1.93 (s, 1H), 1.55 (d, 3H).

### b) 3-Acetyl-5-allyloxy-benzoic acid methyl ester

3-Allyloxy-5-(1-hydroxy-ethyl)-benzoic acid methyl ester (570 mg, 2.41 mmol, 1 eq) is dissolved in DCM (20 ml). Dess-Martin periodane (1.23 g, 2.89 mmol, 1.2 eq) is added to the reaction mixture, and the reaction is stirred for 1 h at room temperature. The reaction mixture is poured into a separating funnel and washed with 1N aq. HCl, followed by brine. The organic layer is dried over sodium sulfate, filtered and concentrated. The residue is purified by column chromatography using EtOAc/hexane in a ratio of 1 to 4 to give the product (475 mg, 84%).

MS (ES+): 252=[M+NH₄]⁺

HPLC (Nucleosil C18HD, 4×70 mm, 3 μm, 20-100% AcCN (6 min), 100% AcCN (1.5 min)) retention time=4.78 min

¹H-NMR (400 MHz, CDCl₃): 8.20 (s, 1H), 7.80 (dd, 1H), 7.69 (dd, 1H), 6.13-6.03 (m, 1H), 5.50-5.34 (m, 2H), 4.63 (d, 2H), 3.95 (s, 3H), 2.62 (s, 3H).

### c) 3-Acetyl-5-allyloxy-benzoic acid

### 3-Acetyl-5-allyloxy-benzoic acid can be obtained by an analogous hydrolysis reaction as for building block A17, starting from 3-acetyl-5-allyloxy-benzoic acid methyl ester

MS (ES+): 238=[M+NH₄]⁺

HPLC (Nucleosil C18HD, 4×70 mm, 3 μm, 20-100% AcCN (6 min), 100% AcCN (1.5 min)) retention time=3.74 min

¹H-NMR (400 MHz, CDCl₃): 8.04 (s, 1H), 7.72-7.67 (m, 2H), 6.13-6.02 (m, 1H), 5.45-5.29 (m, 2H), 4.73 (d, 2H), 2.62 (s, 3H).

### Building block A40: 3-Allyloxy-5-(2-oxo-propyl)-benzoic acid

### a) 3-Allyloxy-5-formyl-benzoic acid methyl ester

3-Allyloxy-5-hydroxymethyl-benzoic acid methyl ester (3.5 g, 15.8 mmol, 1 eq) is dissolved in DCM (250 ml) and Dess-Martin periodane (8.0 g, 18.9 mmol, 1.2 eq) is added. The reaction mixture is stirred for 1 h at room temperature and quenched by the addition of HCl (1 N in water). The organic layer is washed with brine, dried over MgSO₄, filtered and concentrated. The residue is purified by chromatography using hexane/EtOAc in a ratio of 9 to 1 to give the product (3.10 g, 91%).

MS (ES+): 238=[M+NH4]⁺

HPLC (Nucleosil C18HD, 4×70 mm, 3 μm, 20-100% AcCN (6 min), 100% AcCN (1.5 min)) retention time=4.73 min

### b) 3-Allyloxy-5-formyl-benzoic acid

3-Allyloxy-5-formyl-benzoic acid can be obtained by an analogous hydrolysis reaction as for building block A17, starting from 3-allyloxy-5-formyl-benzoic acid methyl ester.

MS (ES+): 224=[M+NH₄]⁺

HPLC (Nucleosil C18HD, 4×70 mm, 3 μm, 20-100% AcCN (6 min), 100% AcCN (1.5 min)) retention time=3.63 min

### c) 3-Allyloxy-5-((E)-2-nitro-propenyl)-benzoic acid

3-Allyloxy-5-formyl-benzoic acid (733 mg, 3.38 mmol, 1 eq) is dissolved in toluene (20 ml). Nitroethane (10 ml) is added, followed by NH₄OAc (312 mg, 4.05 mmol, 1.2 eq). The reaction mixture is refluxed for 3 hours using a Dean-Stark-Trap to remove water. The reaction mixture is filtered and concentrated. The residue is purified by column chromatography using EtOAc/hexane/AcOH in a ratio of 120 to 60 to 1 to give the product (450 mg, 51%).

MS (ES+): 281=[M+NH₄]⁺

HPLC (Nucleosil C18HD, 4×70 mm, 3 μm, 20-100% AcCN (6 min), 100% AcCN (1.5 min)) retention time=4.61 min

¹H-NMR (400 MHz, d6-DMSO): 8.12 (s, 1H), 7.70 (s, 1H), 7.51 (s, 1H), 7.41 (s, 1H), 6.11-6.01 (m, 1H), 5.42-5.28 (m, 2H), 4.69 (d, 2H), 2.40 (s, 3H).

### d) 3-Allyloxy-5-(2-oxo-propyl)-benzoic acid

CrCl₂ (1.77 g, 14.4 mmol, 10 eq) is suspended in 1N HCl in H₂O (10 ml), and 3-allyloxy-5-((E)-2-nitro-propenyl)-benzoic acid (400 mg, 1.44 mmol, 1 eq) is added as solution in THF (10 ml). The reaction mixture is stirred for 16 h at room temperature. The mixture is then diluted with 50 ml of Et₂O, the organic layer is separated and washed with brine, dried over sodiumsulfate, filtered and concentrated to give the product (300 mg, 89%).

MS (ES+): 252=[M+NH₄]⁺

¹H-NMR (400 MHz, CDCl₃): 7.60-7.58 (m, 2H), 7.06 (dd, 1H), 6.15-6.02 (m, 1H), 5.50-5.33 (m, 2H), 4.62 (d, 2H), 3.79 (s, 2H), 2.22 (s, 3H).

### Building block A41: 3-(Allyl-benzyloxycarbonyl-amino)-5-(3-oxo-butyl)-benzoic acid

### a) 3-Benzyloxycarbonylamino-5-(tert-butyl-dimethyl-silanyloxymethyl)-benzoic acid methyl ester

5-Benzyloxycarbonylamino-5-hydroxymethyl-benzoic acid methyl ester (14.3 g, 45.4 mmol, 1 eq) is dissolved in DMF (40 ml). Tert-butylchlorodimethylsilane (8.3 g, 54.9 mmol, 1.21 eq), imidazole (3.1 g, 45.8 mmol, 1.01 eq) and 4-dimethylaminopyridine (279 mg, 2.28 mmol, 0.05 eq) are added. The reaction is stirred for 8 h at room temperature and then diluted with diethyl ether and aqueous sodium bicarbonate. The organic layer is separated, dried over magnesium sulfate, filtered and concentrated. The residue is dissolved in diethyl ether and hexane is added to precipitate the product, which is filtered and dried under vacuum (16.1 g, 83%).

MS (ES+): 447=[M+NH₄]⁺

HPLC (Nucleosil C18HD, 4×70 mm, 3 μm, 20-100% AcCN (6 min), 100% AcCN (1.5 min)) retention time=7.09 min

¹H-NMR (400 MHz, CDCl₃): 7.96 (s, 1H), 7.76 (s, 1H), 7.67 (s, 1H), 7.47-7.37 (m, 5H), 6.82 (s, 1H), 5.26 (s, 2H), 4.80 (s, 2H), 3.93 (s, 3H), 0.99 (s, 9H), 0.18 (s, 6H).

### b) 3-(Allyl-benzyloxycarbonyl-amino)-5-(tert-butyl-dimethyl-sianyloxymethyl)-benzoic acid methyl ester

3-Benzyloxycarbonylamino-5-(tert-butyl-dimethyl-silanyloxymethyl)-benzoic acid methyl ester (14.0 g, 29.8 mmol, 1 eq) is dissolved in 200 ml of DMF and cooled to 0° C. NaH (1.63 g, 55%, 37.3 mmol, 1.25 eq) is added at 0° C., and the reaction is stirred for 1 h at 0° C. Allyl bromide (3.30 ml, 37.3 mmol, 1.25 eq) is added, and the reaction is stirred for 30 min at room temperature. The reaction mixture is poured on ice water and diluted with EtOAc. The organic layer is separated, dried over magnesium sulfate, filtered and concentrated to give the product (15.3 g, 99%).

MS (ES+): 487=[M+NH₄]⁺

HPLC (Nucleosil C18HD, 4×70 mm, 3 μm, 20-100% AcCN (6 min), 100% AcCN (1.5 min)) retention time=7.43 min

¹H-NMR (400 MHz, CDCl₃): 7.86 (s, 1H), 7.82 (s, 1H), 7.49 (s, 1H), 7.40-7.30 (m, 5H), 6.00-5.88 (m, 1H), 5.26-5.17 (s, 4H), 4.80 (s, 2H), 4.38 (d, 2H), 3.93 (s, 3H), 0.99 (s, 9H), 0.18 (s, 6H).

### c) 3-(Allyl-benzyloxycarbonyl-amino)-5-hydroxymethyl-benzoic acid methyl ester

3-(Allyl-benzyloxycarbonyl-amino)-5-(tert-butyl-dimethyl-silanyloxymethyl)-benzoic acid methyl ester (19.8 g, 37.2 mmol, 1 eq) is dissolved in THF (220 ml). TBAF (100 ml, 1 N in THF, 100 mmol, 2.68 eq) is and the reaction is stirred for 16 hours at room temperature. The mixture is concentrated, and the residue is purified by column chromatography using hexane/EtOAc in a ratio of 7 to 3 to give the product (11 g, 70%).

MS (ES+): 373=[M+NH₄]⁺

HPLC (Nucleosil C18HD, 4×70 mm, 3 μm, 20-100% AcCN (6 min), 100% AcCN (1.5 min)) retention time=4.63 min

¹H-NMR (400 MHz, CDCl₃): 7.92 (s, 1H), 7.86 (s, 1H), 7.49 (s, 1H), 7.40-7.30 (m, 5H), 6.00-5.88 (m, 1H), 5.26-5.17 (s, 4H), 4.76 (s, 2H), 4.38 (d, 2H), 3.92 (s, 3H).

### d) 3-(Allyl-benzyloxycarbonyl-amino)-5-bromomethyl-benzoic acid methyl ester

3-(Allyl-benzyloxycarbonyl-amino)-5-hydroxymethyl-benzoic acid methyl ester (1.00 g, 2.81 mmol, 1 eq) is dissolved in DCM (40 ml) and cooled to 0° C. Triphenylphospin (812 mg, 3.09 mmol, 1.1 eq) is added at 0° C., followed by CBr₄ (1.02 g, 3.10 mmol, 1.1 eq). The reaction is stirred for 1 h at 0° C. and 2 h at room temperature. The mixture is concentrated, and the residue is purified by column chromatography using hexane/EtOAc in a ratio of 8 to 2 to give the product (1.06 g, 91%).

MS (ES+): 435 and 437=[M+H]⁺

HPLC (Nucleosil C18HD, 4×70 mm, 3 μm, 20-100% AcCN (6 min), 100% AcCN (1.5 min)) retention time=5.94 min

¹H-NMR (400 MHz, CDCl₃): 7.95 (s, 1H), 7.88 (s, 1H), 7.52 (s, 1H), 7.40-7.30 (m, 5H), 6.00-5.88 (m, 1H), 5.22-5.17 (s, 4H), 4.50 (s, 2H), 4.38 (d, 2H), 3.94 (s, 3H).

### e) 3-(Allyl-benzyloxycarbonyl-amino)-5-(2-methoxycarbonyl-3-oxo-butyl)-benzoic acid methyl ester

Sodium ethanolate (172 mg, 2.53 mmol, 1 eq) is suspended in EtOH (25 ml) and methyl acetoacetate (274 μl, 2.53 mmol, 1 eq) is added. After 30 min 3-(allyl-benzyloxycarbonyl-amino)-5-bromomethyl-benzoic acid methyl ester (1.06 g, 2.53 mmol, 1 eq) dissolved in 5 ml of EtOH is added. The reaction is refluxed for 7 h and then cooled to room temperature. The mixture is diluted with EtOAc and 1 N aq. HCl. The organic layer is separated, dried over sodium sulfate, filtered and concentrated. The residue is purified by column chromatography using EtOAc/hexane in a ratio of 1 to 4 to give the product (345 mg, 30%).

MS (ES+): 471=[M+NH₄]⁺

HPLC (Nucleosil C18HD, 4×70 mm, 3 μm, 20-100% AcCN (6 min), 100% AcCN (1.5 min)) retention time=5.45 min

¹H-NMR (400 MHz, CDCl₃): 7.81 (s, 1H), 7.77 (s, 1H), 7.40-7.27 (m, 6H), 5.99-5.85 (m, 1H), 5.22-5.15 (m, 4H), 4.37 (d, 2H), 3.93 (s, 3H), 3.79 (t, 1H), 3.71 (s, 3H), 3.22-3.18 (m, 2H), 2.20 (s, 3H).

### f) 3-(Allyl-benzyloxycarbonyl-amino)-5-(3-oxo-butyl)-benzoic acid

3-(Allyl-benzyloxycarbonyl-amino)-5-(2-methoxycarbonyl-3-oxo-butyl)-benzoic acid methyl ester (345 mg, 0.76 mmol, 1 eq) is dissolved in EtOH (10 ml). After the addition of 4N aq. NaOH (2.18 ml) and water (10 ml) the reaction is refluxed for 3 h. The mixture is diluted with 1N HCl and EtOAc, the organic layer is separated, dried over magnesium sulfate, filtered and concentrated. The residue is purified by column chromatography using EtOAc to give the product (285 mg, 98%).

MS (ES+): 399 [M+NH₄]⁺

HPLC (Nucleosil C18HD, 4×70 mm, 3 μm, 20-100% AcCN (6 min), 100% AcCN (1.5 min)) retention time=4.13 min

¹H-NMR (400 MHz, CDCl₃): 7.86 (s, 1H), 7.81 (s, 1H), 7.42-7.30 (m, 6H), 6.00-5.88 (m, 1H), 5.23-5.17 (m, 4H), 4.38 (d, 2H), 2.97 (t, 2H), 2.79 (t, 2H), 2.20 (s, 3H).

### Building block A42: 2-Allylamino-6-methoxymethyl-isonicotinic acid

### a) 2-Chloro-6-methyl-1-oxy-isonicotinic acid

2-Chloro-6-methyl-isonicotinic acid (6.86 g, 40 mmol, 1 eq) is dissolved in AcOH (40 ml). 2 ml of hydrogen peroxide (35% in H₂O) is added to the reaction mixture, and the reaction is stirred for 76 hours at 95° C. During the reaction time, 2 ml of hydrogen peroxide (35% in H₂O) are added five times in regular intervals. The reaction mixture is concentrated and coevaporated with toluene to give the product (7.25 g, 96%).

HPLC (Nucleosil C18HD, 4×70 mm, 3 μm, 5-100% AcCN (6 min), 100% AcCN (1.5 min)) retention time=2.46 min

¹H-NMR (400 MHz, d6-DMSO): 8.05 (d, 1H), 7.96 (d, 1H), 2.46 (s, 3H).

### b) 2-Chloro-6-hydroxymethyl-isonicotinic acid

2-Chloro-6-methyl-1-oxy-isonicotinic acid (7.3 g, 39 mmol, 1 eq) is dissolved in acetic acid anhydride, and the reaction mixture is stirred at 100° C. for 2 hours. The reaction mixture is cooled to 40° C., and water (40 ml) is added over 2 hours. The mixture is concentrated and purified by column chromatography using DCM/MeOH/AcOH in a ratio of 360 to 39 to 1 to give the acetylated product (5.8 g, 64%). The acetylated product is dissolved in MeOH (50 ml), and 2N aq. NaOH (25 ml) is added. The reaction is stirred for 4 hours and then diluted with 2N aq. HCl. The mixture is concentrated and then diluted with DCM. The organic layer is separated, dried over sodium sulfate, filtered and concentrated to give the product (4.6 g, 63%).

MS (ES−): 186=[M−H]-HPLC (Nucleosil C18HD, 4×70 mm, 3 μm, 5-100% AcCN (6 min), 100% AcCN (1.5 min)) retention time=2.97 min

### c) 2-Chloro-6-methoxymethyl-isonicotinic acid

2-Chloro-6-hydroxymethyl-isonicotinic acid (4.6 g, 24.5 mmol, 1 eq) is dissolved in 100 ml of DMF. NaH (3.53 g, 73.5 mmol, 3 eq) is added at 0° C. The reaction mixture is stirred for 1 hour at 10° C., then methyl iodide (7.63 ml, 123 mmol, 5 eq) is added within 15 min. The reaction is stirred at room temperature for 4 hours, and then quenched with 10 ml of 4N aq. NaOH. The reaction mixture is diluted with 4N aq. HCl and concentrated. The residue is diluted with DCM/MeOH 9 to 1, and the organic layer is concentrated. The residue is purified by column chromatography using DCM/EtOH/AcOH in a ratio of 180 to 19 to 1 to give the product (3.48 g, 70%).

MS (ES+): 202=[M+H]⁺

HPLC (Nucleosil C18HD, 4×70 mm, 3 μm, 5-100% AcCN (6 min), 100% AcCN (1.5 min)) retention time=3.80 min

### d) 2-Chloro-6-methoxymethyl-isonicotinic acid tert-butyl ester

2-Chloro-6-methoxymethyl-isonicotinic acid (3.48 g, 15.5 mmol, 1 eq) is dissolved in toluene (60 ml) and heated to 80° C. N,N-dimethylformamid-di-tertbutylacetal (7.53 ml, 31 mmol, 2 eq) is added in portions over 8 hours. The reaction mixture is then diluted with TBME and washed with aqueous sodium bicarbonate. The organic layer is dried over sodium sulfate, filtered and concentrated to give the product (2.3 g, 56%).

MS (ES+): 258=[M+H]⁺

HPLC (Nucleosil C18HD, 4×70 mm, 3 μm, 5-100% AcCN (6 min), 100% AcCN (1.5 min)) retention time=6.24 min

### e) 2-Allylamino-6-methoxymethyl-isonicotinic acid tert-butyl ester

Pd(OAc)₂ (97 mg, 0.42 mmol, 0.05 eq), BINAP (269 mg, 0.42 mmol, 0.05 eq), sodium tertbutanolate (1.66 g, 17 mmol, 2 eq), and allylamine (784 mg, 12.7 mmol, 1.5 eq) are dissolved in toluene (80 ml) and stirred at 50° C. for 20 min. 2-Chloro-6-methoxymethyl-isonicotinic acid tert-butyl ester (1.38 g, 5.4 mmol, 1 eq) is dissolved in toluene (20 ml) and added to the reaction mixture at 50° C. within 20 min. The reaction is stirred at 50° C. for 1 h. The reaction mixture is cooled to room temperature and poured on ice and TBME (200 ml). Ammonium chloride (4 g) is added and the mixture is stirred for 20 min. The organic layer is separated, dried over sodium sulfate, filtered and concentrated to give the product (950 mg, 63%).

MS (ES+): 279=[M+H]⁺

HPLC (Nucleosil C18HD, 4×70 mm, 3 μm, 5-100% AcCN (6 min), 100% AcCN (1.5 min)) retention time=4.33 min

¹H-NMR (400 MHz, CDCl₃): 7.18 (s, 1H), 6.87 (s, 1H), 6.02-5.92 (m, 1H), 5.37-5.19 (m, 2H), 4.88-4.82 (m, 1H), 4.47 (s, 2H), 4.01-3.97 (m, 2H), 3.50 (s, 3H), 1.62 (s, 9H).

### f) 2-Allylamino-6-methoxymethyl-isonicotinic acid

2-Allylamino-6-methoxymethyl-isonicotinic acid tert-butyl ester (270 mg, 0.97 mmol, 1 eq) is dissolved in 4N HCl in dioxane (4.9 ml). The reaction is stirred for 83 h at room temperature. The reaction mixture is then concentrated and coevaporated with toluene to give the product (248 mg, 95%).

MS (ES+): 223=[M+H]⁺

HPLC (Nucleosil C18HD, 4×70 mm, 3 μm, 5-100% AcCN (6 min), 100% AcCN (1.5 min)) retention time=2.59 min

### Building block A43: 2-Benzyloxycarbonylamino-6-methyl-isonicotinic acid ethyl ester

### a) 2-(N′-Isopropylidene-hydrazino)-6-methyl-isonicotinic acid ethyl ester

A mixture of 7.35 g 42.86 mmol) 2-chloro-6-methyl-isonicotinic acid, 10.75 g (250 mmol) hydrazine hydrate and 10.7 ml 4N NaOH is stirred at 125° C. for 24 h. The mixture is evaporated to dryness, taken up in 35 ml water, 35 ml ethanol and 50 ml acetone and stirred for 1 h. The mixture is concentrated once more and refluxed in a solution of 20 ml thionyl chloride in 200 ml ethanol. After 1.5 h the mixture is cooled down and filtered. The filtrate is diluted with ethyl acetate and washed with 10% aq. NaHCO₃ solution. The aqueous phase is extracted with EtOAc/acetone (4:1) three times. The combined organic layers are dried over sodium sulfate and chromatographed on silica gel ((EtOAc/hexanes=1:2) to give 9.2 g of a brownish oil, which crystallizes from EtOH/water. Mp 79-82° C.

Rf: (EtOAc/hexanes)=1/1): 0.27

LC (Nucleosil C-18HD, 4×70 mm, 3 μm, 5-100% AcCN (6 min), 100% AcCN (1.5 min)): 3.617 min

MS (ES+): 236=[M+H]⁺

¹H-NMR (400 MHz, CDCl₃): 8.05 (br, 1H), 7.59 (s, 1H), 7.14 (s, 1H), 4.39 (q, 2H), 2.46 (s, 3H), 2.07 (s, 3H), 1.93 (s, 3H), 1.41 (t, 3H).

### b) 2-Amino-6-methyl-isonicotinic acid ethyl ester

A solution of 8.37 g (35.6 mmol) 2-(N′-isopropylidene-hydrazino)-6-methyl-isonicotinic acid ethyl ester in 150 ml EtOH is hydrogenated for 11 h at 80° C. and 6 bar hydrogen in the presence of 25 g Raney-Ni. After cooling down the mixture is filtered over celite and evaporated. The product is crystallized from EtOH/water to give 4.1 g of white crystals.

Rf: (EtOAc/hexanes)=1/1): 0.29

LC (Nucleosil C-18HD, 4×70 mm, 3 μm, 20-100% AcCN (6 min), 100% AcCN (1.5 min)): 1.355 min

MS (ES+): 181=[M+H]⁺

¹H-NMR (400 MHz, CDCl₃): 7.08 (s, 1H), 6.93 (s, 1H), 4.61 (br, 2H), 4.19 (q, 2H), 2.46 (s, 3H), 1.41 (t, 3H).

### c) 2-Benzyloxycarbonylamino-6-methyl-isonicotinic acid ethyl ester

To a stirred mixture of 1.03 g (5.74 mmol) 2-amino-6-methyl-isonicotinic acid ethyl ester and 1.45 g (17.2 mmol) NaHCO₃ in acetonitrile are added dropwise 2.93 ml (8.6 mmol) of a 50% solution of benzyl chloroformate in toluene. After 16 h the mixture is diluted with water, extracted with ethyl acetate, dried over sodium sulfate and chromatographed on silica gel (EtOAc/hexanes=1:5) to give 1.68 g of a colorless solid.

Rf: (EtOAc/hexanes)=1/3): 0.25

LC (Nucleosil C-18HD, 4×70 mm, 3 μm, 20-100% AcCN (6 min), 100% AcCN (1.5 min)): 5.485 min

### Building block A44: 2-(Acetyl-allyl-amino)-6-methyl-isonicotinic acid

The title compound is prepared similarly to building block A27, starting from 2-amino-6-methyl-isonicotinic acid ethyl ester (see building block A43b). The crude product is crystallized from EtOAc/hexanes to obtain a white powder.

LC (Zorbax SB-C18H, 3×30 mm, 1.8 μm, 10-100% MeCN (3.25 min), 100% MeCN (0.75 min), 100-10% MeCN (0.25 min)): 2.354 min

MS (ES+): 235=[M+H]⁺

¹H-NMR (400 MHz, d6-DMSO): 7.73 (s, 1H), 7.59 (s, 1H), 5.93-5.82 (m, 1H), 5.16-5.07 (m, 2H), 4.52-4.46 (m, 2H), 2.53 (s, 3H), 2.07 (s, 3H).

### Building block B1: [(1S*,2S*,4R*)-1-(3-Allyloxy-benzyl)-4-butylcarbamoyl-2-hydroxy-pentyl]-carbamic acid tert-butyl ester

### a) [1-Benzenesulfonyl-2-(3-benzyloxy-phenyl)-ethyl]-carbamic acid tert-butyl ester

A suspension of 20.6 g (91 mmol) (3-benzyloxy-phenyl)-acetaldehyde, 10.7 g (91 mmol, I eq) tert-butylcarbamate, 18.3 g (109 mmol, 1.2 eq) sodium benzenesulfinate and 5.2 ml (137 mmol, 1.5 eq) formic acid in 155 ml acetonitrile is stirred at 80° C. for 4 h. After cooling to rt the mixture is taken up in EtOAc. The solution is washed with bicarbonate and brine, dried over magnesium sulfate and evaporated. The residue (37.3 g) is used for the next step without further purification.

MS (LC/MS): 490=[M+Na]⁺

### b) [(S*)-2-(3-Benzyloxy-phenyl)-1-((S*)-5-oxo-2,5-dihydro-furan-2-yl)-ethyl]-carbamic acid tert-butyl ester

To a solution of 80 ml (commercial 2 M solution in THF/heptane/ethylbenzene, 160 mmol, 2 eq) lithium diisopropylamide in 180 ml THF is added slowly at −78° C. a solution of 11.2 ml (160 mmol, 2 eq) 5H-furan-2-one in 60 ml THF. The mixture is stirred for another 20 min at −78° C. before a solution of 37.3 g (80 mmol) [1-benzenesulfonyl-2-(4-benzyloxy-phenyl)-ethyl]-carbamic acid tert-butyl ester in 220 ml THF is added at the same temperature. After stirring for another 45 min at −78° C. aq. bicarbonate solution is added and the reaction mixture is taken up into EtOAc. The organic layer is washed with bicarbonate and brine and dried over magnesium sulfate. Evaporation of the solvent gives a residue that is purified by chromatography on silica using hexan/EtOAc 9/1 to 7/3. The product is recrystallized from ether/hexane to give 11.1 g (27 mmol, 30% over two steps) of the product as white crystals.

MS (LC/MS): 432=[M+Na]⁺

¹H-NMR (400 MHz, CDCl₃): 7.45-7.2 (m, 7H), 6.9-6.85 (m, 3H), 6.06 (d, 1H), 5.07 (s, 2H), 4.90 (d, 1H), 4.50 (d, 1H), 4.20 (q, 1H), 3.01 (dd, 1H), 2.91 (dd, 1H), 1.38 (s, 9H).

### c) [(S*)-2-(3-Benzyloxy-phenyl)-1-((S*)-5-oxo-tetrahydro-furan-2-yl)-ethyl-carbamic acid tert-butyl ester

A solution of 11.1 g (27 mmol) [(S*)-2-(4-benzyloxy-phenyl)-1-((S*)-5-oxo-2,5-dihydro-furan-2-yl)-ethyl]-carbamic acid tert-butyl ester in 550 ml THF is hydrogenated (1 atm H₂) at rt with 2.3 g Pt/C as catalyst (5% Engelhard 4709) during 1 h. The catalyst is filtered off and the filtrate is evaporated. Purification by chromatography on silica (Flashmaster, hexane to hexane/EtOAc 55/45 over 40 min) gives 10.4 g (25 mmol, 94%) of the product as yellowish oil.

MS (LC/MS): 434=[M+Na]⁺

¹H-NMR (400 MHz, CDCl₃): 7.45-7.2 (m, 6H), 6.9-6.8 (m, 3H), 5.06 (s, 2H), 4.61 (d, 1H), 4.44 (t, 1H), 4.00 (q, 1H), 2.95 (dd, 1H), 2.85 (dd, 1H), 2.6-2.45 (m, 2H), 2.15-2.1 (m, 2H), 1.42 (s, 9H).

### d) [(S*)-2-(3-Benzyloxy-phenyl)-1-((2S*,4R*)-4-methyl-5-oxo-tetrahydro-furan-2-yl)-ethyl]-carbamic acid tert-butyl ester

To a solution of 11.4 g (27.7 mmol) [(S*)-2-(4-benzyloxy-phenyl)-1-((S*)-5-oxo-2,5-dihydro-furan-2-yl)-ethyl]-carbamic acid tert-butyl ester in 35 ml THF and 5 ml (42 mmol, 1.5 eq) DMPU is added dropwise at −78° C. 55 ml (1 M solution in THF, 55 mmol, 2 eq) lithium-bis-(trimethylsilyl)-amide. After stirring at −78° C. for another 45 min methyliodide is added dropwise and the mixture is stirred another 3 h at −78° C. 10.3 ml (138 mmol, 5 eq) Propionic acid are added followed by 10 ml water. After warming up to 0° C. 72 ml of a 10% solution of citric acid is added. The reaction mixture is extracted with EtOAc. The organic layer is washed with bicarbonate, 0.1 N sodium sulfite and brine, dried over magnesium sulfate and evaporated. Purification by chromatography on silica (hexane/EtOAc 9/1 to 4/1) followed by recrystallization from ether/hexane gives 8.14 g (19 mmol, 69%) white crystals.

MS (LC/MS): 448=[M+Na]⁺

¹H-NMR (400 MHz, CDCl₃): 7.45-7.2 (m, 6H), 6.9-6.8 (m, 3H), 5.05 (s, 2H), 4.53 (d, 1H), 4.45 (t, 1H), 4.00 (q, 1H), 2.93-2.85 (m, 2H), 2.74-2.68 (m, 1H), 2.41-2.34 (m, 1H), 1.89-1.82 (m, 1H), 1.41 (s, 9H), 1.26 (d, 3H).

### e) [(1S*,2S*,4R*)-1-(3-Benzyloxy-benzyl)-4-butylcarbamoyl-2-hydroxy-pentyl]-carbamic acid tert-butyl ester

A solution of 4.0 g (9.4 mmol) [(S*)-2-(3-benzyloxy-phenyl)-1-((2S*,4R*)-4-methyl-5-oxo-tetrahydro-furan-2-yl)-ethyl]-carbamic acid tert-butyl ester in 200 ml n-butylamine is stirred for 18 h in an heating bath of 90° C. The n-butylamine is evaporated and the residue is recrystallized from DCM/ether/hexane to give 4.42 g (8.8 mmol, 94%) of white crystals.

MS (LC/MS): 521=[M+Na]⁺

¹H-NMR (400 MHz, CDCl₃): 7.45-7.15 (m, 6H), 6.9-6.8 (m, 3H), 5.91 (s, 1H), 5.04 (s, 2H), 4.89 (d, 1H), 3.7-3.6 (m, 2H), 3.3-3.1 (m, 2H), 2.9-2.85 (m, 2H), 2.6-2.5 (m, 1H), 1.75-1.6 (m, 2H), 1.5-1.25 (m, 4H), 1.41 (s, 9H), 1.12 (d, 3H), 0.92 (t, 3H).

### f) [(1S*,2S*,4R*)-4-Butylcarbamoyl-2-hydroxy-1-(3-hydroxy-benzyl)-pentyl]-carbamic acid tert-butyl ester

A solution of 4.55 g (9.1 mmol) [(1S*,2S*,4R*)-1-(3-benzyloxy-benzyl)-4-butylcarbamoyl-2-hydroxy-pentyl]-carbamic acid tert-butyl ester in 570 ml ethanol is hydrogenated (1 atm H₂) at rt with 1.14 g Pd/C (10% Engelhard 4505) for 1 h. Filtration through glassfibers and evaporation of the solvent followed by chromatography on silica (flashmaster, DCM to DCM/MeOH 4/1) gives 3.67 g (8.9 mmol, 98%) of the product as a white foam.

MS (LC/MS): 431=[M+Na]⁺

¹H-NMR (400 MHz, CDCl₃): 7.44 (S, 1H), 7.11 (t, 1H), 6.74-6.70 (m, 3H), 6.11 (t, 1H), 5.06 (d, 1H), 4.25 (br s, 1H), 3.75-3.55 (m, 2H), 3.28-3.10 (m, 2H), 2.9-2.8 (m, 2H), 2.60-2.50 (m, 1H), 1.75-1.60 (m, 2H), 1.47-1.26 (m, 4H), 1.40 (s, 9H), 1.11 (d, 3H), 0.90 (t, 3H).

### g) [(1S*,2S*,4R*)-1-(3-Allyloxy-benzyl)-4-butylcarbamoyl-2-hydroxy-pentyl]-carbamic acid tert-butyl ester

A mixture of 0.60 g (1.47 mmol) [(1S*,2S*,4R*)-4-butylcarbamoyl-2-hydroxy-1-(3-hydroxy-benzyl)-pentyl]-carbamic acid tert-butyl ester, 0.186 ml (2.2 mmol, 1.5 eq) allyl bromide, 0.609 g (4.4 mmol, 3 eq) water free potassium carbonate and 0.244 g (1.47 mmol, 1 eq) potassium iodide in 150 ml dry acetone are heated at reflux temperature during 3 days. The reaction is diluted with EtOAc, washed with brine, dried over sodium sulfate, and the solvents are evaporated at reduced pressure. The product is purified by chromatography on silica (flashmaster, DCM to DCM/MeOH 9/1) and recrystallization from ether/hexane to give 0.56 g (1.26 mmol, 85%) of the product.

MS (LC/MS): 571=[M+Na]⁺

¹H-NMR (400 MHz, CDCl₃): 7.22 (t, 1H), 6.86-6.78 (m, 3H), 6.14-6.04 (m, 1H), 5.80 (br s, 1H), 5.44 (dd, 1H), 5.31 (dd, 1H), 4.90 (d, 1H), 4.55 (d, 2H), 3.94 (br s, 1H), 3.74-3.66 (m, 2H), 3.33-3.17 (m, 2H), 2.90 (br d, 2H), 2.60-2.52 (m, 1H), 1.77-1.61 (m, 2H), 1.52-1.31 (m, 4H), 1.43 (s, 9H), 1.15 (d, 3H), 0.95 (t, 3H).

### Building block B2: [(S*)-2-(3-Allyl-phenyl)-1-((2S*,4R*)-4-methyl-5-oxo-tetrahydro-furan-2-yl)-ethyl]-carbamic acid tert-butyl ester

### a) [(S*)-2-(3-Hydroxy-phenyl)-1-((2S*,4R*)-4-methyl-5-oxo-tetrahydro-furan-2yl)-ethyl]-carbamic acid tert-butyl ester

A solution of 1.02 g (2.4 mmol) [(S*)-2-(3-Benzyloxy-phenyl)-1-((2S*,4R*)-4-methyl-5-oxo-tetrahydro-furan-2-yl)-ethyl]-carbamic acid tert-butyl ester (see building block B1d) in 75 ml ethanol is hydrogenated (1 atm H₂) at rt with 0.2 g Pd/C (10% Engelhard 4505) for 0.5 h. Filtration through a bed of Hyflo and evaporation of the solvent gives 0.8 g (2.4 mmol, 100%) of the product as a white foam.

MS (LC/MS): 358=[M+Na]⁺

¹H-NMR (400 MHz, CDCl₃): 7.16 (t, 1H), 6.77 (d, 1H), 6.75-6.70 (m, 2H), 4.61 (d, 1H), 4.52-4.45 (m, 1H), 4.00 (q, 1H), 2.9-2.8 (m, 2H), 2.77-2.67 (m, 1H), 2.39-2.34 (m, 1H), 1.90-1.83 (m, 1H), 1.41 (s, 9H), 1.26 (d, 3H).

### b) Trifluoro-methanesulfonic acid 3-[(S*)-2-tert-butoxycarbonylamino-2-((2S*,4R*)-4-methyl-5-oxo-tetrahydro-furan-2-yl)-ethyl]-phenyl ester

A mixture of 0.95 g (2.8 mmol) [(S*)-2-(3-Hydroxy-phenyl)-1-((2S*,4R*)-4-methyl-5-oxo-tetrahydro-furan-2-yl)-ethyl]-carbamic acid tert-butyl ester, 1.01 g (2.8 mmol, 1.0 eq) N-phenyl-bis-(trifluoromethanesulfinimide) (CAS 37595-74-7) and 1.17 g (8.5 mmol, 3 eq) waterfree potassium carbonate in 10 ml dry THF is heated in a microwave apparatus at 120° C. for 1.5 h. The mixture is diluted with EtOAc and washed with brine. Drying over magnesium sulfate and evaporation of the solvent followed by purification by chromatography on silica (flashmaster, hexane to hexane/EtOAc 60/40) gives 1.12 g (2.4 mmol, 86%) of the product.

MS (LC/MS): 490=[M+Na]⁺

¹H-NMR (400 MHz, CDCl₃): 7.44 (t, 1H), 7.32 (d, 1H), 7.21-7.19 (m, 2H), 4.57 (d, 1H), 4.52 (t, 1H), 4.06 (q, 1H), 2.99 (d, 2H), 2.81-2.71 (m, 1H), 2.44-2.38 (m, 1H), 1.97-1.90 (m, 1H), 1.40 (s, 9H), 1.31 (d, 3H).

### c) [(S*)-2-(3-Allyl-phenyl)-1-((2S*,4R*)-4-methyl-5-oxo-tetrahydro-furan-2-yl)-ethyl]-carbamic acid tert-butyl ester

To a solution of 1.01 g (2.16 mmol) trifluoro-methanesulfonic acid 3-[(S*)-2-tert-butoxycarbonylamino-2-((2S*,4R*)-4-methyl-5-oxo-tetrahydro-furan-2-yl)-ethyl]-phenyl ester in dry DMF are added under an argon atmosphere 0.75 ml (2.38 mmol, 1.1 eq) but-3-enyl-tributyl-stannane, 0.23 g (5.4 mmol, 2.5 eq) waterfree lithiumchloride and 30 mg (0.04 mmol, 1.9 mol %) bis-(triphenylphosphin)-palladium(II)-chloride and the mixture is stirred at 100° C. bath temperature for 45 min. After cooling to rt the mixture is diluted with EtOAc, washed with brine, dried over magnesium sulfate and the solvents are evaporated. The residue is purified by chromatography on silica (flashmaster, hexane to hexane/EtOAc 50/50) followed by crystallization from ether/hexane to give 0.64 g (1.78 mmol, 82%) of the product as white crystals.

MS (LC/MS): 382=[M+Na]⁺

¹H-NMR (400 MHz, CDCl₃): 7.27 (t, 1H), 7.13-7.07 (m, 3H), 6.04-5.93 (m, 1H), 5.13 (d, 1H), 5.09 (s, 1H), 4.75-4.50 (m, 2H), 4.03 (q, 1H), 3.40 (d, 2H), 2.96-2.87 (m, 2H), 2.80-2.70 (m 1H), 2.45-2.39 (m, 1H), 1.93-1.86 (m, 1H), 1.42 (s, 9H), 1.29 (d, 3H).

### Building block B3: [(S)-2-(3-Allyl-phenyl)-1-((2S,4R)-4-methyl-5-oxo-tetrahydro-furan-2-yl)-ethyl]-carbamic acid tert-butyl ester

The enantiomers of [(S*)-2-(3-Benzyloxy-phenyl)-1-((2S*,4R*)-4-methyl-5-oxo-tetrahydro-furan-2-yl)-ethyl]-carbamic acid tert-butyl ester (buildingblock B1d) are separated by preparative chiral HPLC to give [(S)-2-(3-benzyloxy-phenyl)-1-((2S,4R)-4-methyl-5-oxo-tetrahydro-furan-2-yl)-ethyl]-carbamic acid tert-butyl ester (α_{D}=+10°, c=1 in CHCl3). Derivatization according to example buildingblock B2a-c gives [(S)-2-(3-Allyl-phenyl)-1-((2S,4R)-4-methyl-5-oxo-tetrahydro-furan-2-yl)-ethyl]-carbamic acid tert-butyl ester.

MS (LC/MS): 382=[M+Na]⁺

¹H-NMR (400 MHz, CDCl₃): 7.27 (t, 1H), 7.12-7.07 (m, 3H), 6.04-5.94 (m, 1H), 5.13 (d, 1H), 5.09 (s, 1H), 4.60-4.50 (m, 2H), 4.03 (q, 1H), 3.40 (d, 2H), 2.96-2.87 (m, 2H), 2.80-2.70 (m 1H), 2.45-2.38 (m, 1H), 1.93-1.86 (m, 1H), 1.42 (s, 9H), 1.29 (d, 3H).

### Building block C1: (S)-3-(3-Benzyloxy-phenyl)-2-tert-butoxycarbonylamino-propionic acid methyl ester

The non-natural amino acid building block C is prepared by methods disclosed in the literature and known to those skilled in the art. For example, Tetrahedron 2002, 58, 6951-6963, J. Am. Chem. Soc. 1993, 115, 10125-10138.

m.p.: 80-81° C.

[α]_{D}²²+39.1° (c=1.29, CHCl₃)

Rf: (DCM/EtOAc=90/10): 0.69

MS (ES+): 408=[M+Na]⁺

¹H-NMR (400 MHz, d6-DMSO): 7.45-7.29 (m, 5H), 7.27 (d, 1H), 7.18 (t, 1H), 6.89 (s, 1H), 6.87-6.81 (m, 1H), 6.79 (d, 1H), 5.06 (s, 2H), 4.21-4.14 (m, 1H), 3.60 (s, 3H), 2.99-2.92 (m, 1H), 2.84-2.77 (m, 1H), 1.33 (s, 9H).

### Building block C2: (S)-3-(3-Bromo-phenyl)-2-tert-butoxycarbonylamino-propionic acid methyl ester

The title compound is prepared similarly to building block C1, using 3-bromo-benzaldehyde instead of 3-benzyloxy-benzaldehyde.

m.p.: 60-61° C.

[α]_{D}²²+50.8° (c=1.00, CHCl₃)

Rf: (DCM/EtOAc=90/10): 0.54

MS (ES+): 380=[M+Na]⁺

¹H-NMR (400 MHz, d6-DMSO): 7.44 (s, 1H), 7.41-7.36 (m, 1H), 7.30 (d, 1H), 7.23 (d, 2H), 4.23-4.15 (m, 1H), 3.62 (s, 3H), 3.04-2.98 (m, 1H), 2.87-2.79 (m, 1H), 1.32 (s, 9H).

### Building block C3: (S)-3-(3-Allyloxy-phenyl)-2-tert-butoxycarbonylamino-propionic acid methyl ester

### a) (S)-2-tert-Butoxycarbonylamino-3-(3-hydroxy-phenyl)-propionic acid methyl ester

A solution of 5.81 g (15 mmol) (S)-3-(3-Benzyloxy-phenyl)-2-tert-butoxycarbonylamino-propionic acid methyl ester (building block C1) in 150 ml EtOH is stirred at rt in the presence of 1.5 g 10% Pd/C under a hydrogen atmosphere for 2 h. The catalyst is filtered off and the filtrate evaporated to give 4.68 g of the desired product as colorless solid. This is used for the next step without further purification.

m.p.: 61-65° C.

Rf: (DCM/EtOAc=80/20): 0.34

MS (ES+): 318=[M+Na]⁺

¹H-NMR (400 MHz, d6-DMSO): 9.27 (s, 1H), 7.22 (d, 1H), 7.04 (t, 1H), 6.63-6.56 (m, 3H), 4.15-4.07 (m, 1H), 3.60 (s, 3H), 2.91-2.84 (m, 1H), 2.79-2.71 (m, 1H), 1.33 (s, 9H).

### b) (S)-3-(3-Allyloxy-phenyl)-2-tert-butoxycarbonylamino-propionic acid methyl ester

To a solution of 2.34 g (7.5 mmol) (S)-2-tert-Butoxycarbonylamino-3-(3-hydroxy-phenyl)-propionic acid methyl ester in 15 ml acetone is added 1.25 g (9.75 mmol) powdered K₂CO₃ and 0.76 ml (9 mmol) allylbromide, the mixture is stirred for 16 h at 80° C. 15 ml Water is added and the mixture is extracted with DCM (2×15 ml). The combined organic layers are washed with 7.5 ml 1 M sodium hydroxide, 7.5 ml halfsaturated sodium chloride, dried over sodium sulfate and evaporated to give 2.49 g of the desired product as colorless solid.

m.p.: 50-51° C.

[α]_{D}²²+40.9° (c=1.18, CHCl₃)

Rf: (DCM/EtOAc=80120): 0.70

MS (ES+): 358=[M+Na]⁺

¹H-NMR (400 MHz, d6-DMSO): 7.25 (d, 1H), 7.17 (t, 1H), 6.83-6.75 (m, 3H), 6.08-5.97 (m, 1H), 5.40-5.34 (m, 1H), 5.26-5.21 (m, 1H), 4.52 (d, 2H), 4.19-4.12 (m, 1H), 3.61 (s, 3H), 2.98-2.92 (m, 1H), 2.84-2.77 (m, 1H), 1.33 (s, 9H).

### Building block C4: (S)-3-(3-Allyl-phenyl)-2-tert-butoxycarbonylamino-propionic acid methyl ester

A solution of 4.21 g (11.75 mmol) (S)-3-(3-Bromo-phenyl)-2-tert-butoxycarbonylamino-propionic acid methyl ester (building block C2), 5.58 ml (17.6 mmol) allyltributyltin and 1.51 g (35.3. mmol) lithium chloride in 118 ml dimethylamide is degassed. Under argon atmosphere 367 mg (0.59 mmol) SK-CC02-A are added and the mixture warmed to 100° C. for 17 h. After addition of 41 ml saturated potassium fluoride solution at 0° C. the mixture is stirred at rt for 30 min the resulting suspension is filtered and washed with EtOAc (3×59 ml). The layers of the filtrate are separated, the aqueous phase is extracted with 179 ml EtOAc, the combined organic layers are washed with water, dried over sodium sulfate and evaporated. The residue is purified by chromatography on silica gel (cyclohexane/EtOAc 90/10) and gives 1.95 g of the desired product as yellow oil.

Rf: (cyclohexane/EtOAc=80/20): 0.31

MS (ES+): 342.1=[M+Na]⁺

¹H-NMR (400 MHz, d6-DMSO): 7.26 (d, 1H), 7.19 (t, 1H), 7.06-6.99 (m, 3H), 5.98-5.87 (m, 1H), 5.18-5.00 (m, 2H), 4.18-4.10 (m, 1H), 3.59 (s, 3H), 3.32 (d, 2H), 2.98-2.91 (m, 1H), 2.87-2.79 (m, 1H), 1.32 (s, 9H).

### Building block C5: {(1S,2R)-1-(3-Allyl-benzyl)-3-[benzyloxycarbonyl-(3-isopropyl-benzyl)-amino]-2-hydroxy-propyl}-carbamic acid tert-butyl ester

### a) [(S)-1-(3-Allyl-benzyl)-3-chloro-2-oxo-propyl]-carbamic acid tert-butyl ester

A solution of 1.95 g (6.1 mmol) (S)-3-(3-Allyl-phenyl)-2-tert-butoxycarbonylamino-propionic acid methyl ester (building block C4) in 61 ml THF is cooled to −78° C. and 1.8 ml (24.4 mmol) chloroiodomethane is added. 20.8 ml (30.5 mmol) of a 1.47 M THF solution of LDA are added dropwise while the temperature of the reaction mixture is maintained below −73° C., and the mixture is stirred for an additional 30 min. The reaction is carefully quenched with 9.1 ml (159 mmol) glacial acetic acid while the temperature is maintained below −65° C. After stirring for 15 min at −78° C. the mixture is allowed to warm to 0° C. and 92 ml of a half-saturated aqueous sodium chloride solution is added. The mixture is extracted with TBME (2×92 ml), the combined organic layers are washed with 92 ml 1 M sodium sulfite and 92 ml water, dried over sodium sulfate and evaporated. The 3.2 g of the desired product are used for the next step without further purification.

Rf: (cyclohexane/EtOAc=80/20): 0.34

MS (LC/MS): 359.8=[M+Na]⁺

### b) [(1S,2S)-1-(3-Allyl-benzyl)-3-chloro-2-hydroxy-propyl]-carbamic acid tert-butyl ester

A solution of 471 mg (12.2 mmol) sodium borohydride in 44 ml EtOH is cooled to −78° C., a solution of 3.2 g (6.1 mmol) [(S)-1-(3-Allyl-benzyl)-3-chloro-2-oxo-propyl]-carbamic acid tert-butyl ester in 90 ml ethanol is added dropwise maintaining the internal temperature below −75° C. At −78° C. stirring is continued for 1 h, then the mixture is allowed to warm to rt within 17 h. At −78° C. 31 ml of 1 M HCl are added dropwise, the mixture is allowed to warm to rt. Ethanol is evaporated and the residual aqueous solution is extracted with EtOAc (2×61 ml). The combined organic layers are washed with 61 ml halfsaturated sodium chloride solution, dried over sodium sulfate and evaporated. The residue is purified by chromatography on silica gel (cyclohexane/EtOAc 90/10 to 80/20) and gives 1.51 g of the desired product as pale brown solid.

m.p.: 123-126° C.

Rf: (cyclohexane/EtOAc=80/20): 0.19

MS (ES+): 362.2=[M+H]⁺

¹H-NMR (400 MHz, d6-DMSO): 7.15 (t, 1H), 7.04-6.94 (m, 3H), 6.67 (d, 1H), 5.97-5.87 (m, 1H), 5.40 (d, 1H), 5.09-4.99 (m, 2H), 3.68-3.52 (m, 3H), 3.49-3.43 (m, 1H), 3.00-2.94 (m, 1H), 2.58-2.52 (m, 1H), 1.28 (s, 9H).

### c) [(S)-2-(3-Allyl-phenyl)-1-(S)-oxiranyl-ethyl]-carbamic acid tert-butyl ester

A solution of 3.20 g (10.5 mmol) [(1S,2S)-1-(3-Allyl-benzyl)-3-chloro-2-hydroxy-propyl]-carbamic acid tert-butyl ester in a mixture of 19 ml THF, 19 ml MeOH and 21 ml (21 mmol, 2 eq) 1 N aq sodium hydroxide is stirred at rt for 3 h. The reaction is diluted with 80 ml saturated aq. ammonium chloride solution and extracted with DCM. The organic layer is dried over sodium sulfate and the solvents are evaporated to give 2.81 g (9.3 mmol, 88%) of the product.

MS (LC/MS): 326=[M+Na]⁺

¹H-NMR (400 MHz, CDCl₃): 7.27 (d, 1H), 7.12-7.08 (m, 3H), 6.05-5.94 (m, 1H), 5.13 (d, 1H), 5.09 (s, 1H), 4.47 (br s, 1H), 3.71 (br s, 1H), 3.41 (d, 2H), 3.01-2.78 (m, 5H), 1.42 (s, 9H).

### d) [(1S,2R)-1-(3-Allyl-benzyl)-2-hydroxy-3-(3-isopropyl-benzylamino)-propyl]-carbamic acid tert-butyl ester

A solution of 2.81 g (9.3 mmol) [(S)-2-(3-Allyl-phenyl)-1-(S)-oxiranyl-ethyl]-carbamic acid tert-butyl ester and 1.80 g (12.0 mmol, 1.3 eq) 3-isopropyl-benzylamine in 50 ml ethanol is heated (bath temperature 50° C.) and stirred over night. The excess amine is evaporated and the residue is purified by chromatography on silica (flashmaster, hexane to hexane/EtOAc 1/1) to give 2.57 g (5.7 mmol, 61%) of the product.

MS (LC/MS): 453=[M+H]⁺

¹H-NMR (400 MHz, CDCl₃): 7.31-7.23 (m, 2H), 7.20-7.16 (m, 3H), 7.1-7.07 (m, 3H), 6.04-5.94 (m, 1H), 5.12 (d, 1H), 5.08 (s, 1H), 4.73 (d, 1H), 3.86 (br s, 1H), 3.84 (d, 1H), 3.79 (d, 1H), 3.54 (q, 1H), 3.40 (d, 2H), 3.00-2.84 (m, 4H), 2.80-2.73 (m, 2H), 1.40 (s, 9H), 1.29 (d, 6H).

### e) {(1S,2R)-1-(3-Allyl-benzyl)-3-[benzyloxycarbonyl-(3-isopropyl-benzyl)-amino]-2-hydroxy-propyl}-carbamic acid tert-butyl ester

To a solution of 2.57 g (5.67 mmol, 1 eq) [(1S,2R)-1-(3-allyl-benzyl)-2-hydroxy-3-(3-isopropyl-benzylamino)-propyl]-carbamic acid tert-butyl ester in 100 ml DCM and 50 ml saturated aq. sodium carbonate are added 3.80 ml (50% in toluene, 11.4 mmol, 2 eq) benzyl chloroformate. The reaction is stirred at rt for 2.5 h and then diluted with EtOAc, washed with brine, 0.1 N aq. HCl, aq. sodium bicarbonate and brine again, dried over sodium sulfate and the solvents are evaporated at reduced pressure. The residue is purified by chromatography on silica (flashmaster, hexane to hexane/EtOAc 3/2) to give 2.59 g (4.4 mmol, 78%) of the product.

MS (LC/MS): 609=[M+Na]⁺

¹H-NMR (300 MHz, CDCl₃): 7.4-7.3 (m, 6H), 7.23-7.17 (m, 2H), 7.11 (d, 1H), 7.05-6.9 (m, 5H), 6.01-5.87 (m, 1H), 5.21 (s, 2H), 5.08 (d, 1H), 5.03 (s, 1H), 4.53 (br s, 2H), 3.76 (br s, 2H), 3.45-3.3 (m, 2H), 3.35 (d, 2H), 2.95-2.75 (m, 3H), 1.34 (s, 9H), 1.20 (d, 6H).

### Building block C6: {(1S,2R)-1-(3-Allyloxy-benzyl)-3-[benzyloxycarbonyl-(3-isopropylbenzyl)-amino]-2-hydroxy-propyl}-carbamic acid tert-butyl ester

The title compound is obtained by an analogous reaction sequence as for building block C5, starting from (S)-3-(3-Allyloxy-phenyl)-2-tert-butoxycarbonylamino-propionic acid methyl ester (building block C3).

MS (ES+): 603=[M+H]⁺

¹H-NMR (400 MHz, CDCl₃): 7.43-7.10 (m, 10H), 7.09-7.00 (m, 1H), 6.85-6.80 (m, 2H), 6.15-6.03 (m, 1H), 5.42 (d, 1H), 5.31 (d, 1H), 5.27-5.20 (m, 1H), 4.62-4.50 (m, 3H), 4.44-4.38 (m, 1H), 3.85-3.75 (m, 2H), 3.57-3.40 (m, 2H), 3.00-2.80 (m, 2H), 1.40-1.20 (m, 15H).

### Building block C7: {(1S,2R)-1-(3-Allyloxy-benzyl)-3-[benzyloxycarbonyl-(4-isopropyl-pyridin-2-ylmethyl)-amino]-2-hydroxy-propyl}-carbamic acid tert-butyl ester

The title compound is obtained by an analogous reaction sequence as for building block C5, starting from (S)-3-(3-allyloxy-phenyl)-2-tert-butoxycarbonylamino-propionic acid methyl ester (building block C3) and C-(4-isopropyl-pyridin-2-yl)-methylamine (building block D1).

MS (ES+): 342=[M+H+Na]⁺

¹H-NMR (400 MHz, CDCl₃): 8.48 (d, 1H), 7.21 (t, 1H), 7.14 (s, 1H), 7.08 (d, 1H), 6.86-6.79 (m, 3H), 6.13-6.04 (m, 1H), 5.44 (d, 1H), 5.31 (d, 1H), 4.68 (d, 1H), 4.55 (d, 2H), 3.99 (d, 1H), 3.94 (d, 1H), 3.93-3.83 (m, 1H), 3.6-3.53 (m, 1H), 3.00-2.75 (m, 6H), 1.38 (s, 9H), 1.29 (d, 6H).

### Building block C8: {(1S,2R)-1-(3-Allyl-benzyl)-3-[benzyloxycarbonyl-(4-isopropyl-pyridin-2-ylmethyl)-amino]-2-hydroxy-propyl}-carbamic acid tert-butyl ester

The title compound is obtained by an analogous reaction sequence as for building block C5, starting from (S)-3-(3-allyl-phenyl)-2-tert-butoxycarbonylamino-propionic acid methyl ester (building block C4) and C-(4-isopropyl-pyridin-2-yl)-methylamine (building block D1).

MS (ES+): 610=[M+H+Na]⁺

¹H-NMR (400 MHz, d6-DMSO, 363K): 8.38 (d, 1H), 7.3-7.2 (m, 5H), 7.17-6.97 (m, 6H), 6.17 (br s, 1H), 6.02-5.92 (m, 1H), 5.30 (d, 1H), 5.09 (s, 2H), 5.03 (d, 1H), 4.65 (d, 1H), 4.58 (d, 1H), 3.80-3.74 (m, 1H), 3.65-3.56 (m, 2H), 3.32 (d, 2H), 3.32-3.25 (m, 1H), 3.03-2.97 (m, 1H), 2.91-2.81 (m, 1H), 2.62 (dd, 1H), 1.27 (s, 9H), 1.18 (d, 6H).

### Building block C9: {Acetoxy-[(R)-3-(3-isopropyl-benzyl)-2-oxo-oxazolidin-5-yl]-methyl}-carbamic acid tert-butyl ester

### a) (R)-3-(3-Isopropyl-benzylamino)-propane-1,2-diol

A mixture of 5.89 g (64.7 mmol) (R)-3-amino-propane-1,2-diol, 9.58 g (64.7 mmol) 3-isopropyl-benzaldehyde in 30 ml toluene and 30 ml cyclohexane containing 1 ml EtOH is refluxed with azeotropic removal of water. The residual clear solution is concentrated in vacuum, dissolved in 200 ml EtOH and cooled to +4° C. A solution of 4.9 g NaBH₄ in 10 ml water is slowly added so that the reaction temperature does not exceed +10° C. The mixture is stirred at 25° C. for 16 h. Excess hydride is quenched through the dropwise addition of 55 ml 4N HCl. After 1 h the mixture is evaporated to dryness. After addition of toluene/EtOH this is repeated twice. The residue is taken up in 200 ml EtOH, filtered and concentrated in vacuo to yield 16.6 g of the hydrochloride salt of the title compound as a colorless oil, which is used in the next step without purification.

LC (Nucleosil C-18HD, 4×70 mm, 3 μm, 20-100% AcCN (6 min), 100% AcCN (1.5 min)): 2.503 min MS (ES) MH⁺=224

¹H-NMR (400 MHz, CD₃OD): 7.43-7.34 (m, 4H), 4.25 (s, 2H), 4.00-3.93 (m, 1H), 3.65-3.54 (m, 2H), 3.22-2.94 (m, 3H), 1.28 (d, 6H).

### b) ((R)-2,3-Dihydroxy-propyl)-(3-isopropyl-benzyl)-carbamic acid methyl ester

(R)-3-(3-isopropyl-benzylamino)-propane-1,2-diol hydrochloride (16.6 g, ca. 64 mmol) is stirred at +4° C. in 120 ml 10% aq. Na₂CO₃ and 100 ml DCM. Methyl chloroformate (4.92 ml, 64 mmol) is added over a period of 10 minutes. While warming up the stirring is continued for 2 h. The mixture is extracted with four portions of 50 ml DCM, dried over Na₂SO₄ and evaporated to yield 17.1 g of the title compound as a colorless oil.

Rf: (EtOAc): 0.47

LC (Nucleosil C-18HD, 4×70 mm, 3 μm, 20-100% AcCN (6 min), 100% AcCN (1.5 min)): 4.023 min

MS (ES) [M+23]⁺=304

### c) (R)-5-Hydroxymethyl-3-(3-isopropyl-benzyl)-oxazolidin-2-one

To a solution of 1.55 g Na (67.4 mmol) in 200 ml MeOH is added 17.1 g (60.8 mmol) ((R)-2,3-dihydroxy-propyl)-(3-isopropyl-benzyl)-carbamic acid methyl ester in 50 ml MeOH. The mixture is refluxed for 1 h, cooled down and neutralized with 3.7 g (69 mmol) NH₄Cl. Brine and water is added and the mixture is extracted with DCM four times. The combined organic layers are dried and evaporated, taken up in TBME and filtered over high-flow. The filtrate is crystallized from TBME/hexane to yield 11.58 g of the title compound as white crystals.

Mp.: 75-77° C.

Rf: (EtOAc/hexane=1:1): 0.19

LC (Nucleosil C-18HD, 4×70 mm, 3 μm, 20-100% AcCN (6 min), 100% AcCN (1.5 min)): 4.052 min

MS (ES) [M+23]⁺⁼272

### d) (Hydroxy-[(R)-3-(3-isopropyl-benzyl)-2-oxo-oxazolidin-5-yl]-methyl}-carbamic acid tert-butyl ester

To a −78° C. solution of 6.67 ml (77.7 mmol) oxalyl chloride in DCM are added dropwise 11.1 ml (156.3 mmol) DMSO. After 20 minutes is added a solution of 11.6 g (46.52 mmol) (R)-5-hydroxymethyl-3-(3-isopropyl-benzyl)-oxazolidin-2-one in 45 ml DCM, after 30 minutes followed by 19.45 ml (139.58 mmol) triethylamine. The mixture is warmed to 0° C. and stirred for 30 minutes. The clear solution is diluted with TBME, washed with brine, dried over Na₂SO₄ and concentrated in vacuo with the bath temperature below 30° C. The crude (R)-3-(3-isopropyl-benzyl)-2-oxo-oxazolidine-5-carbaldehyde (Rf: (EtOAc): 0.39) is treated immediately with 5.99 g (51.17 mmol) carbamic acid tert-butyl ester, 9.16 g (55.82 mmol) sodium benzene sulfonate and 2.63 ml (70 mmol) formic acid in 150 ml acetonitrile. The mixture is stirred for 48 h, diluted with EtOAc, washed with brine, dried and chromatographed on silica gel (hexane/EtOAc 2:1, 1:1). Yield 12.5 g of {hydroxy-[(R)-3-(3-isopropyl-benzyl)-2-oxo-oxazolidin-5-yl]-methyl}-carbamic acid tert-butyl ester as a resin.

Rf: (EtOAc/hexane=1:1): 0.35

LC (Nucleosil C-18HD, 4×70 mm, 3 μm, 20-100% AcCN (6 min), 100% AcCN (1.5 min)): 4.922 min

MS (ES) [M+23]⁺⁼387

### e) (Acetoxy-[(R)-3-(3-isopropyl-benzyl)-2-oxo-oxazolidin-5-yl]-methyl}-carbamic acid tert-butyl ester

A solution of 11.5 g (31.55 mmol) {hydroxy-[(R)-3-(3-isopropyl-benzyl)-2-oxo-oxazolidin-5-yl]-methyl}-carbamic acid tert-butyl ester in 80 ml pyridine and 40 ml of acetic anhydride is kept at 25° C. for 16 h. The mixture is concentrated in vacuo. The residue is taken up in xylene and evaporated. This procedure is repeated six times to yield 11.2 g of the title compound as a yellowish solid (1:1 mixture of diastereomers)

Rf: (EtOAc/toluene =1:2): 0.42, 0.46

LC (Nucleosil C-18HD, 4×70 mm, 3 μm, 20-100% AcCN (6 min), 100% AcCN (1.5 min)): 5.476 and 5.557 min

MS (ES) [M+23]⁺⁼429

1H-NMR (400 MHz, CDCl3): 7.35-7.08 (m, 4H), 6.28 and 6.20 (both dd, 1H), 5.6 and 5.45 (both br, 1H), 4.78 (br, 1H), 4.53-4.32 (2 AB, 2H), 3.54 (dt, 1H) 3.28-3.21 (m, 1H), 2.92 (heptet, 1H), 2.10 and 1.94 (both s, 3H), 1.44 8d, 6H), 1.27 and 1.24 (both s, 3H).

### Building block C10: {(S)-2-(3-Allyloxy-4-methoxy-phenyl)-1-[(R)-3-(3-isopropyl-benzyl)-2-oxo-oxazolidin-5-yl]-ethyl}-carbamic acid tert-butyl ester

In a three-necked flask of 250 ml, equipped with reflux condensor, thermometer and rubber septum are stirred under vacuum 1.36 g (56 mmol) magnesium turnings at 100° C. for 1 h. To the cooled flask is added 10 ml THF under nitrogen atmosphere. The magnesium turnings are activated by addition of 100 mg dibromoethane. Immediately thereafter a solution of 8.0 g (38 mmol) 2-allyloxy-4-chloromethyl-1-methoxy-benzene (buidling block F1) in 50 ml dry THF is added dropwise over a period of 1 h, so that a reaction temperature of 60° C. is maintained. The clear Grignard solution is transfered by canula and positive nitrogen pressure to a 250 ml flask kept under nitrogen and cooled down to −75° C. A solution of 4.63 g (11.4 mmol) acetoxy-[(R)-3-(3-isopropyl-benzyl)-2-oxo-oxazolidin-5-yl]-methyl}-carbamic acid tert-butyl ester (building block C9) in 10 ml THF is added within 30 minutes and stirring is continued at −75° C. for 1 h. The mixture is poured onto 10% aq. ammonium chloride and extracted with EtOAc. The organic phase is washed with water and brine, dried and concentrated. Chromatography on silica gel using a gradient of EtOAc/hexane 1:4 to 1:1 gave, besides side products and a diastereoisomer, 2.39 g of {(S)-2-(3-allyloxy-4-methoxy-phenyl)-1-[(R)-3-(3-isopropyl-benzyl)-2-oxo-oxazolidin-5-yl]-ethyl}-carbamic acid tert-butyl ester as a colorless oil.

Rf: (hexane/EtOAc=1/1): 0.71

LC (Nucleosil C-18HD, 4×70 mm, 3 μm, 40-100% AcCN (6 min), 100% AcCN (1.5 min)): 5.24 min

MS (ES+): 425=[M+H]⁺

### Building block C11: (S)-5-[(S)-2-(3-Allyl-4-methoxy-phenyl)-1-methyl-ethyl]-3-(3-isopropyl-benzyl)-oxazolidin-2-one

The title compound is obtained by an analogous reaction sequence as for building block C10, starting from {acetoxy-[(R)-3-(3-isopropyl-benzyl)-2-oxo-oxazolidin-5-yl]-methyl}-carbamic acid tert-butyl ester (building block C9) and 2-allyl-4-chloromethyl-1-methoxy-benzene (building block F2).

LC (Nucleosil C-18HD, 4×70 mm, 3 μm, 20-100% AcCN (6 min), 100% AcCN (1.5 min)): 6.618 min

MS (ES−): 507=[M−H]

¹H-NMR (400 MHz, CDCl₃): 7.33-6.95 (m, 6H), 6.91 (d, 1H), 6.07-5.96 (m, 1H), 5.10-5.06 (m, 2H), 4.61-4.25 (m, 4H), 3.98-3.88 (m, 1H), 3.93 (s, 3H), 3.42 (t, 1H), 3.39 (d, 2H), 3.30-3.26 (m, 1H), 2.95-2.80 (m, 3H), 1.38 (br s, 9H), 1.23 (d, 6H).

### Building block C12: {(S)-2-(3-Allyl-phenyl)-1-[(R)-3-(3-isopropyl-benzyl)-2-oxo oxazolidin-5-yl]-ethyl}-carbamic acid tert-butyl ester

The title compound is obtained as yellowish resin by an analogous reaction sequence as for building block C10, starting from {acetoxy-[(R)-3-(3-isopropyl-benzyl)-2-oxo-oxazolidin-5-yl]-methyl}-carbamic acid tert-butyl ester (building block C9) and 1-allyl-3-chloromethyl-benzene (building block F3).

Rf: (EtOAc/hexanes)=113): 0.18

LC (Nucleosil C-18HD, 4×70 mm, 3 μm, 65-100% MeCN (6 min), 100% MeCN (1.5 min)): 3.406 min

MS (ES+): 501=[M+Na]⁺

### Building block C13: [(1S,2R)-1-(3-Allyloxy-5-methyl-benzyl)-2-hydroxy-3-(3-isopropyl-benzylamino)-propyl]-carbamic acid tert-butyl ester

The title compound is obtained as yellowish resin by an analogous reaction sequence as for building block C10, starting from {acetoxy-[(R)-3-(3-isopropyl-benzyl)-2-oxo-oxazolidin-5-yl]-methyl}-carbamic acid tert-butyl ester (building block C9) and 1-allyloxy-3-chloromethyl-5-methyl-benzene (building block F4).

Rf: (EtOAc/hexanes)=1/1): 0.51

LC (Nucleosil C-18HD, 4×70 mm, 3 μm, 65-100% MeCN (6 min), 100% MeCN (1.5 min)): 3.507 min

MS (ES+): 531=[M+Na]⁺

### Building block C14: {(1S,2R)-1-(3-Allyl-benzyl)-3-[benzyloxycarbonyl-(4-tert-butyl-pyridin-2-ylmethyl)-amino]-2-hydroxy-propyl}-carbamic acid tert-butyl ester

The title compound is obtained by an analogous reaction sequence as for building block C5, starting from (S)-3-(3-allyl-phenyl)-2-tert-butoxycarbonylamino-propionic acid methyl ester (building block C4) and C-(4-tert-butyl-pyridin-2-yl)-methylamine (building block D5).

MS (ES+): 602=[M+H]⁺

¹H-NMR (400 MHz, CDCl₃): 8.46/8.43 (d, 1H), 7.34-7.20 (m, 11H), 6.04-5.92 (m, 1H), 5.14-4.96 (m, 4H), 4.75-4.42 (m, 3H), 0.94-3.79 (m, 3H), 3.42-3.33 (m, 3H), 3.13-3.02 (m, 1H), 2.98-2.88 (m, 1H), 1.63 (br s, 1H), 1.37/1.24 (s, 9H), 1.36 (s, 9H).

### Building block C15: {(S)-2-(3-Benzyloxy-phenyl)-1-[(R)-3-(3-isopropyl-benzyl)-2-oxo-oxazolidin-5-yl]-ethyl}-carbamic acid tert-butyl ester

The title compound is obtained as yellowish resin by an analogous reaction sequence as for building block C10, starting from {acetoxy-[(R)-3-(3-isopropyl-benzyl)-2-oxo-oxazolidin-5-yl]-methyl}-carbamic acid tert-butyl ester (building block C9) and 1-benzyloxy-3-chloromethyl-benzene (Tetrahedr. 1969, 25, 4011).

Rf: (EtOAc/hexanes)=1/3): 0.18

LC (Nucleosil C-18HD, 4×70 mm, 3 μm, 65-100% AcCN (6 min), 100% AcCN (1.5 min)): 3.795 min

MS (ES+): 567=[M+Na]⁺

### Building block C16: {(S)-2-(3-Hydroxy-phenyl)-1-[(R)-3-(3-isopropyl-benzyl)-2-oxo-oxazolidin-5-yl]-ethyl}-carbamic acid tert-butyl ester

A solution of 1.3 g (2.4 mmol) {(S)-2-(3-benzyloxy-phenyl)-1-[(R)-3-(3-isopropyl-benzyl)-2-oxo-oxazolidin-5-yl]-ethyl}-carbamic acid tert-butyl ester (building block C15) in 50 ml ethanol is hydrogenated (1 atm H₂) at rt with 0.5 g Pd/C (10% Engelhard 4505) for 10 h. Filtration through celite and evaporation of the solvent followed by chromatography on silica (Ee/hexanes 1:1) gives 1.03 g of the product as a colorless resin.

Rf: (EtOAc/hexanes)=1/1): 0.39

LC (Nucleosil C-18HD, 4×70 mm, 3 μm, 20-100% AcCN (6 min), 100% AcCN (1.5 min)): 5.430 min

### Building block C17: [(S)-2-(3-Hydroxy-phenyl)-1-((R)-2-oxo-oxazolidin-5-yl)-ethyl]-carbamic acid tert-butyl ester

At a temperature below −40° C., NH₃ gas is condensed into a solution of 4.5 g (8.3 mmol) {(S)-2-(3-benzyloxy-phenyl)-1-[(R)-3-(3-isopropyl-benzyl)-2-oxo-oxazolidin-5-yl]-ethyl}-carbamic acid tert-butyl ester (building block C15) in 20 ml THF. Small chips of Li-metal (267 mg, 38.2 mmol) are added, while the temperature is maintained between −40 and −33° C. When a dark blue color persists the reaction is quenched with 2.4 g solid ammonium chloride and allowed to warm to rt slowly. The mixture is diluted with EtOAc, washed with brine, dried over sodium sulfate and chromatographed on silica gel (EtOAc).

Yield 2.42 g of a colorless resin.

Rf: (EtOAc): 0.45

LC (Zorbax SB-C18H, 3×30 mm, 1.8 μm, 30-100% AcCN (3.25 min), 100% AcCN (0.75 min), 100-30% AcCN (0.25 min)): 0.718 min

MS (ES+): 667=[2M+Na]⁺

### Building block C18: [(S)-2-(3-Allyloxy-phenyl)-1-((R)-2-oxo-oxazolidin-5-yl)-ethyl]-carbamic acid tert-butyl ester

To a solution of 2.42 g (7.52 mmol) [(S)-2-(3-hydroxy-phenyl)-1-((R)-2-oxo-oxazolidin-5-yl)-ethyl]-carbamic acid tert-butyl ester (building block C17), 2.62 g (10 mmol) triphenylphosphine and 0.614 ml allyl alcohol in 20 ml THF at +4° C. are added 1.74 ml (9 mmol) diisopropyl azodicarboxylate. The mixture is stirred overnight at rt. The mixture is evaporated. Crystallisation from TBME/hexanes removes part of the triphenylphosphine oxide. The mother liquor is chromatographed on silica gel (EtOAc/toluene=1:1) to give a white solid slightly contaminated with triphenylphosphine oxide.

Rf: (EtOAc/hexanes=1:1): 0.13

LC (Zorbax SB-C18H, 3×30 mm, 1.8 μm, 30-100% AcCN (3.25 min), 100% AcCN (0.75 min), 100-30% AcCN (0.25 min)): 2.276 min

MS (ES+): 747=[2M+Na]⁺

### Building block D1: C-(4-Isopropyl-pyridin-2-yl)-methylamine

### a) 4-Isopropyl-pyridine-1-oxide

4-Isopropylpyridine (50 g, 412 mmol, 1 eq) is dissolved in AcOH (450 ml) and H₂O₂ (42 ml, 30% in H₂O, 412 mmol). The reaction mixture is refluxed for 4 hours and then concentrated. The residue is dissolved in DCM and washed with H₂O and aq NaHCO₃. The organic layer is dried over Na₂SO₄, filtered and concentrated to give the product (53.2 g, 387 mmol, 94%).

MS: 138 (M+1)

HPLC (Nucleosil C18HD, 20-100% AcCN): retention time=2.90 min

### b) 4-Isopropyl-pyridine-2-carbonitrile

4-Isopropyl-pyridine-1-oxide (26.6 g, 194 mmol, 1 eq) is dissolved in trimethylsilylcyanide (73 ml, 582 mmol, 3 eq) and triethylamine (59 ml, 427 mmol, 2.2 eq). The reaction is stirred at 100° C. for 2 hours. The reaction mixture is concentrated, and the residue is purified by column chromatography using EtOAc/hexane in a ratio of 1/19 to give the product (26.8 g, 182 mmol, 94%).

HPLC (Nucleosil C18HD, 20-100% AcCN): retention time=4.84 min

MS: 147 (M+1)

¹H-NMR (400 MHz, CDCl₃): 8.60 (s, 1H), 7.60 (s, 1H), 7.40 (s, 1H), 3.01-2.90 (m, 1H), 1.25 (d, 6H).

### c) C-(4-Isopropyl-pyridin-2-yl)-methylamine

LAH (20.3 g, 534 mmol, 1.5 eq) is suspended in THF (710 ml) and stirred at 0° C. 4-Isopropyl-pyridine-2-carbonitrile (52 g, 356 mmol, 1 eq) is dissolved in THF (180 ml) and added to the suspension within 30 min. The reaction is stirred at 0° C. for 1 hour. The reaction is quenched by the addition of aq Na₂SO₄ (270 ml). The reaction mixture is filtered, and diluted with EtOAc. The organic layer is dried over Na₂SO₄, filtered and concentrated. The residue is dissolved in EtOAc, and HCl (1N in EtOAc) is added to give the product as a hydrochloride salt (24.8 g, 132 mmol, 37%).

HPLC (Nucleosil C18HD, 20-100% AcCN): retention time=2.65 min

MS: 151 (M+1).

¹H-NMR (400 MHz, d6-DMSO): 8.80-8.65 (br s, 2H), 8.60 (d, 1H), 7.80 (s, 1H), 7.56 (d, 1H), 4.27 (d, 2H), 3.08-2.98 (m, 1H), 1.23 (d, 6H).

### Building block D2: 3-Cyclopropyl-benzylamine

### a) 3-Cyclopropyl-benzonitrile

To a solution of 9.35 g (40 mmol) 3-iodo-benzonitrile in 240 ml toluene are added 4.47 g (52 mmol, 1.3 eq.) cyclopropyl boronic acid, 26.8 g (120 mmol, 3 eq.) potassium phosphate and 12 ml water then the mixture is degassed under a stream of argon. The catalyst SK-CC02-A (250 mg, 0.4 mmol, 0.01 eq.) is added and the mixture is heated to 100° C. for 17 h. 400 ml water are added, the mixture is extracted with EtOAc (2×400 ml) the combined organic layers are washed with 400 ml water, dried over sodium sulfate and evaporated. The residue is purified by chromatography on silica gel (cyclohexane/EtOAc 80/20) and gives 7.65 g of the desired product as brownish oil.

Rf: (cyclohexane/EtOAc=80/20): 0.48.

### b) 3-Cyclopropyl-benzylamine

A solution of 7.64 g (40 mmol) 3-cyclopropyl-benzonitrile in 400 ml MeOH is stirred at rt in the presence of 20 g Raney-Ni under a hydrogen atmosphere for 2 h. The catalyst is filtered off and the filtrate evaporated. The residue is dissolved in 160 ml 1 M HCl and extracted with DCM (2×160 ml). The acidic aqueous phase is basified with 4 M aqueous ammonia and extracted with DCM (2×160 ml). The combined organic layers are washed with 160 ml water, dried over sodium sulfate and evaporated to give 2.93 g of the desired product as colorless oil.

Rf: (DCM/MeOH=90/10): 0.17

MS (ES+): 148=[M+H]⁺

¹H-NMR (400 MHz, d6-DMSO): 8.23 (br s, 2H), 7.28-7.14 (m, 3H), 7.10-7.05 (m, 1H), 3.94 (s, 2H), 1.95-1.87 (m, 1H), 1.00-0.92 (m, 2H), 0.73-0.66 (m, 2H).

### Building block D3: C-(6-Ethyl-pyrimidin-4-yl)-methylamine

### a) 4-Chloro-6-ethyl-pyrimidine

4-Chloro-6-ethyl-pyrimidine is prepared as described by M. Butters, J. Heterocyclic Chem. 1992, 29, 1369-1370.

### b) 6-Ethyl-pyrimidine-4-carbonitrile

A solution of 414 mg (2.9 mmol) 4-chloro-6-ethyl-pyrimidine in 3 ml toluene is cooled to 0° C. and 1 g (16.9 mmol, 5.8 eq.) trimethylamine is condensed into the solution. After stirring at rt for 62 h the reaction mixture is filtered and the precipitate is washed with Et₂O. The precipitate is dissolved in 3 ml DCM, a solution of 525 mg (3.19 mmol, 1.1 eq.) tetraethyl ammonium cyanide in 3 ml DCM is added dropwise and the mixture stirred at rt for 1 h. The reaction mixture is extracted with ice water (3×15 ml), the combined aqueous phases are extracted with DCM (2×10 ml) and the combined organic phases are dried over sodium sulfate and evaporated. The residue is purified by chromatography on silica gel (pentane/Et₂O 95/5 to 80/20) to give 190 mg of the desired product as a colorless oil.

Rf: (cyclohexane/EtOAc=80/20): 0.19

MS (LC/MS): 134=[M+H]⁺

¹H-NMR (400 MHz, CDCl₃): 9.25 (s, 1H), 7.55 (s, 1H), 2.92 (q, 2H), 1.38 (t, 3H).

### c) C-(6-Ethyl-pyrimidin-4-yl)-methylamine

A solution of 1.33 g (10 mmol) 6-ethyl-pyrimidine-4-carbonitrile in 100 ml glacial acetic acid is stirred at rt in the presence of 213 mg 10% Pd/C under a hydrogen atmosphere for 1 h. The catalyst is filtered off and the filtrate evaporated. The residue is dissolved in 30 ml water and extracted with DCM (2×20 ml), the aqueous phase is basified to pH 14 by addition of NaOH and extracted with DCM/chloroform (1:1) (2×30 ml). The combined DCM/chloroform layers are dried over sodium sulfate and evaporated to give 1.31 g of the desired product as a red oil.

Rf: (DCM/MeOH/NH₃=80/18/2): 0.42

MS (ES+): 138=[M+H]⁺

¹H-NMR (400 MHz, CDCl₃): 9.06 (s, 1H), 7.24 (s, 1H), 3.97 (s, 2H), 2.81 (q, 2H), 1.77 (br s, 2H), 1.34 (t, 3H).

### Building block D4: 3-tert-Butyl-benzylamine

### a) Trifluoromethanesulfonic acid 3-tert-butyl-phenyl ester NVP—

To an ice-cold solution of 10.0 g (65 mmol) 3-tert-Butylphenol in 50 ml pyridine is slowly added 33.3 mL (198 mmol) Tf₂O. After stirring overnight at rt the mixture is poured onto ice-water (800 mL) and extracted with Et₂O. After drying with MgSO₄ the solvent is removed in vacuo and the residue is purified by chromatography on silica gel (hexane/EtOAc 95/5) to give 16.1 g of the desired product as a colorless oil.

t_{R}(HPLC, Nucleosil C18 column, 20-100% CH₃CN/H₂O/6 min, 100% CH₃CN/1.5 min, 100-20% CH₃CN/H₂O/0.5 min, flow: 1.0 ml/min): 6.20 min.

Rf: (hexane/AcOEt=95/5): 0.75

¹H-NMR (400 MHz, CDCl₃): 7.44-7.39 (m, 2H), 7.24-7.23 (m, 1H), 7.11 (d, 1H), 1.38 (s, 9H).

### b) 3-tert-Butyl-benzonitrile

A mixture of 2.0 g (7.1 mmol) trifluoromethanesulfonic acid 3-tert-butyl-phenyl ester, 1.0 g (8.5 mmol) zinc cyanide, and 0.41 g (0.35 mmol) [Pd(PPh)₃]₄ in 24 ml DMF is degassed for 10 min in an ultrasonic bath and heated overnight at 80° C. After cooling to rt, the reaction is quenched with water and extracted with EtOAc. The organic phase is washed with brine, dried over Na₂SO₄ and the solvent is removed in vacuo. The residue is purified by chromatography on silica gel (hexane/EtOAc 95/5) to give 1.1 g of the desired product as a yellow oil.

t_{R}(HPLC, Nucleosil C18 column, 20-100% AcCN/H₂O/6 min, 100% CH₃CN/1.5 min, 100-20% CH₃CN/H₂O/0.5 min, flow: 1.0 ml/min): 5.19 min.

Rf: (hexane/AcOEt=95/5): 0.39

¹H-NMR (400 MHz, CDCl₃): 7.70 (d, 1H), 7.63 (d, 1H), 7.50 (d, 1H), 7.41 (dd, 1H), 1.39 (s, 9H).

### c) 3-tert-Butyl-benzylamine

A mixture of 0.84 g (5.1 mmol) 3-tert-butyl-benzonitrile, 1 mL (25% aq. NH₃) and 0.1 g Raney-Nickel is hydrogenated at 40° C. After completion of reaction, the catalyst is filtered off and washed with MeOH. The solvent is removed in vacuo and the residue is purified by chromatography on silica gel (DCM/MeOH 90/10) to give 0.84 g of the desired product as a green oil.

t_{R} (HPLC, Nucleosil C18 column, 20-100% CH₃CN/H₂O/6 min, 100% CH₃CN/1.5 min, 100-20% CH₃CN/H₂O/0.5 min, flow: 1.0 ml/min): 2.81 min.

Rf: (hexane/AcOEt=95/5): 0.26

MS (ES+): 164=[M+H]⁺

¹H-NMR (400 MHz, CDCl₃): 7.40-7.33 (m, 3H), 7.20-7.18 (m, 1H), 3.90 (s, 2H), 1.60 (bs, 2H), 1.39 (s, 9H).

### Building block D5: C-(4-tert-Butyl-pyridin-2-yl)-methylamine

The title compound is obtained by an analogous reaction sequence as for building block D1, starting from 4-tert-butyl-pyridine.

HPLC (Nucleosil C18HD, 4×70 mm, 3 μm, 5-100% MeCN (6 min), 100% MeCN (1.5 min)) retention time=2.98 min

MS (ES+): 165=[M+H]⁺

¹H-NMR (400 MHz, d6-DMSO): 8.62 (d, 1H), 7.90 (s, 1H), 7.61 (d, 1H), 4.28 (d, 2H), 1.31 (s, 9H).

### Building block D6: 4-tert-Butyl-pyridine-2-carbaldehyde

To a solution of 8.5 g (51.5 mmol) (4-tert-butyl-pyridin-2-yl)-methanol (J. Med. Chem. 1998, 41, 1777) in 250 ml EtOAc at rt is added 44.5 g (515 mmol) MnO₂. The black suspension is stirred at 50° C. for 4 h. The mixture is filtered over celite and evaporated. Chromatography on silica gel (EtOAc/hexanes=1:3) gives 5.93 g of an orange liquid.

Rf: (EtOAc/hexanes=1/3): 0.40

¹H-NMR (400 MHz, CDCl₃): 10.12 (s, 1H), 8.73 (d, 1H), 8.02 (s, 1H), 7.55 (d, 1H), 1.39 (s, 9H).

### Building block E1: (S)-4-(S)-Oxiranyl-2-oxo-11,16-dioxa-3-aza-tricyclo[15.3.1.1*6,10*]docosa-1(21),6,8,10(22),17,19-hexaene-19-carboxylic acid dimethylamide

### a) (2S,3S)-4-(3-Allyloxy-phenyl)-3-amino-1-chloro-butan-2-ol hydrochloride

A solution of 2.23 g (6.26 mmol) [1(S)-(2-chloro-1(S)-hydroxy-ethyl)-3(R)-methyl-hept-6-enyl]-carbamic acid tert-butyl ester [prepared from (S)-3-(3-allyloxy-phenyl)-2-tert-butoxycarbonylamino-propionic acid methyl ester (building block C3) in an analogous manner as described for building block C5b] in 63 ml DCM is cooled to 0° C. and 12.6 ml 5 M HCl in Et₂O (62.6 mmol) is added. The mixture is stirred at rt for 1.5 h. The solvent is evaporated and the residue is crystallized from Et₂O to give 1.73 g of the desired product as pale brownish crystals.

m.p.: 132-135° C.

Rf: (DCM/MeOH/NH₃=90/9/1): 0.39

MS (ES+): 256=[M+H]⁺

¹H-NMR (400 MHz, d6-DMSO): 7.79 (br s, 3H), 7.24 (t, 1H), 6.90-6.71 (m, 3H), 6.09-5.98 (m, 2H), 5.39 (dd, 1H), 5.25 (dd, 1H), 4.56 (d, 2H), 3.95-3.89 (m, 1H), 3.71 (dd, 1H), 3.56-3.47 (m, 2H), 2.93 (dd, 1H), 2.72 (dd, 1H).

### b) 5-Allyloxy-N-[(1S,2S)-1-(3-allyloxy-benzyl)-3-chloro-2-hydroxy-propyl]-N′,N′-di-methyl-isophthalamide

To a solution of 810 mg (3.25 mmol) 5-allyloxy-N,N-dimethyl-isophthalamic acid (building block A29) in 16 ml DCM is added at 0° C. 704 mg (4.55 mmol, 1.4 eq.) HOBt and 763 mg (3.9 mmol, 1.2 eq.) EDC.HCl. After stirring for 10 min 950 mg (3.25 mmol) (2S,3S)-4-(3-allyloxy-phenyl)-3-amino-1-chloro-butan-2-ol hydrochloride and 0.568 ml (3.25 mmol) DIPEA are added, the mixture is allowed to warm to rt and stirring is continued for 16 h. The mixture is diluted with 10 ml DCM and 3 ml EtOH and washed with 1 M soda (2×25 ml), 1 M HCl (2×25 ml) and 25 ml water, dried over sodium sulfate and evaporated. The residue is purified by chromatography on silica gel (cyclohexane/EtOAc 60/40 to 25/75) to give 1.27 g of the desired product as a pale yellow solid.

Rf: (DCM/MeOH/NH₃=90/9/1): 0.55

MS (ES+): 487/489=[M+H]⁺

¹H-NMR (400 MHz, d6-DMSO): 8.52 (d, 1H), 7.58 (s, 2H), 7.35 (t, 1H), 7.30 (s, 1H), 7.08-7.01 (m, 2H), 6.97-6.91 (m, 1H), 6.33-6.16 (m, 2H), 5.81 (d, 1H), 5.67-5.40 (m, 4H), 4.86 (d, 2H), 4.72-4.62 (m, 2H), 4.46-4.37 (m, 1H), 4.03-3.93 (m, 2H), 3.79 (dd, 1H), 3.33-3.26 (m, 1H), 3.23 (s, 3H), 3.11 (s, 3H), 3.07-3.00 (m, 1H).

### c) (S)-4-((S)-2-Chloro-1-hydroxy-ethyl)-2-oxo-11,16-dioxa-3-aza-tricyclo[15.3.1.1*6,10*]docosa-1(21),6,8,10(22),13,17,19-heptaene-19-carboxylic acid dimethylamide

A solution of 672 mg (1.38 mmol) 5-allyloxy-N-[(1S,2S)-1-(3-allyloxy-benzyl)-3-chloro-2-hydroxy-propyl]-N′,N′-di-methyl-isophthalamide in 8 ml DCM is added dropwise within an hour to a refluxing solution of 59 mg (0.069 mmol, 0.05 eq.) [1,3-bis-(2,4,6-trimethylphenyl)-2-imidazolidinylidene)dichloro(phenylmethylene)-(tricyclohexylphosphine)ruthenium] (Grubbs II catalyst) in 65 ml DCM. The mixture is refluxed for additional 30 min then 0.4 ml butylvinylether is added and stirring is continued for 30 min. The reaction mixture is directly loaded on a column and purified by chromatography on silica gel (DCM/MeOH 99/1 to 98/2), to give 443 mg of the desired product as a grayish foam.

Rf: (DCM/MeOH=95/5): 0.21

MS (ES+): 459/461=[M+H]⁺

¹H-NMR (400 MHz, d6-DMSO): 8.18 (d, 1H), 7.21-7.18 (m, 2H), 7.11 (t, 1H), 7.00-6.97 (m, 1H), 6.81 (d, 1H), 6.73-6.68 (m, 2H), 5.94-5.87 (m, 1H), 5.68-5.59 (m, 2H), 4.86-4.68 (m, 3H), 4.66-4.58 (m, 1H), 4.16-4.07 (m, 1H), 3.80-3.72 (m, 2H), 3.55 (dd, 1H), 3.04 (dd, 1H), 2.95 (s, 3H), 2.86 (s, 3H), 2.72 (dd, 1H).

### d) (S)-4-((S)-2-Chloro-1-hydroxy-ethyl)-2-oxo-11,16-dioxa-3-aza-tricyclo[15.3.1.1*6,10*]docosa-1(21),6,8,10(22),17,19-hexaene-19-carboxylic acid dimethylamide

A solution of 643 mg (1.4 mmol) (S)-4-((S)-2-chloro-1-hydroxy-ethyl)-2-oxo-11,16-dioxa-3-aza-tricyclo[15.3.1.1*6,10*]docosa-1(21),6,8,10(22),13,17,19-heptaene-19-carboxylic acid dimethylamide in 25 ml EtOH is stirred at rt in the presence of 200 mg 10% Pd/C under a hydrogen atmosphere for 30 min. The catalyst is filtered off, the filtrate is evaporated and the residue is purified by chromatography on silica gel (DCM to DCM/MeOH 97/3), to give 536 mg of the desired product as a colorless solid.

Rf: (DCM/MeOH=95/5): 0.17

MS (ES+): 461/463=[M+H]⁺

¹H-NMR (400 MHz, d6-DMSO): 8.15 (d, 1H), 7.18 (t, 1H), 7.11-7.05 (m, 2H), 6.97-6.93 (m, 2H), 6.82 (d, 1H), 6.77 (dd, 1H), 5.56 (d, 1H), 4.41-4.32 (m, 1H), 4.24-4.10 (m, 2H), 4.04-3.95 (m, 2H), 3.78-3.68 (m, 2H), 3.55 (q, 1H), 2.99 (dd, 1H), 2.95 (s, 3H), 2.86 (s, 3H), 2.74 (dd, 1H), 1.87-1.66 (m, 4H).

### e) (S)-4-(S)-Oxiranyl-2-oxo-11,16-dioxa-3-aza-tricyclo[15.3.1.1*6,10*]docosa-1(21),6,8,10(22),17,19-hexaene-19-carboxylic acid dimethylamide

To a solution of 507 mg (1.1 mmol) (S)-4-((S)-2-chloro-1-hydroxy-ethyl)-2-oxo-11,16-dioxa-3-aza-tricyclo[15.3.1.1*6,10*]docosa-1(21),6,8,10(22),17,19-hexaene-19-carboxylic acid dimethylamide in 15 ml THF/DCM/MeOH (1:1:1) is added at 0° C. 2.2 ml 1 M sodium hydroxide and the reaction mixture is stirred at 0° C. for 3.5 h. Then 40 ml half saturated ammonium chloride solution is added and the mixture is extracted with DCM (2×50 ml), the combined organic layers are washed with 50 ml water, dried over sodium sulfate and evaporated to give 501 mg of the desired product as a colorless oil.

Rf: (DCM/MeOH=95/5): 0.30

MS (ES+): 425=[M+H]⁺

¹H-NMR (400 MHz, d6-DMSO): 8.32 (d, 1H), 7.18 (t, 1H), 7.10-7.04 (m, 3H), 6.96-6.93 (m, 1H), 6.83-6.80 (m, 1H), 6.77 (dd, 1H), 4.42-4.34 (m, 1H), 4.27-4.18 (m, 2H), 4.02-3.94 (m, 1H), 3.85-3.77 (m, 1H), 3.08-3.03 (m, 1H), 3.00-2.92 (m, 4H), 2.85 (s, 3H), 2.80-2.73 (m, 2H), 1.91-1.61 (m, 4H).

### Building block F1: 2-Allyloxy-4-chloromethyl-1-methoxy-benzene

### a) (3-Allyloxy-4-methoxy-phenyl)-methanol

A mixture of 5-hydroxymethyl-2-methoxy-phenol (20.0 g, 145 mmol), allyl bromide (22.8 g, 188 mmol) and potassium carbonate (40.4 g, 289 mmol) in 100 ml acetone is stirred at 25° C. for 18 h. The mixture is evaporated, taken up in water and extracted 3 times with DCM, dried over Na₂SO₄ and evaporated. The crude product is used in the next step without purification.

Yield 27.5 g of yellow oil.

Rf: (hexane/EtOAc=3/1): 0.04

LC (Nucleosil C-18HD, 4×70 mm, 3 μm, 20-100% AcCN (6 min), 100% AcCN (1.5 min)): 2.88 min

MS (ES+): 177=[M-OH]⁺

¹H-NMR (400 MHz, CDCl₃): 7.0-6.86 (m, 3H), 6.20-6.08 (m, 1H), 5.43 (d, 1H), 5.32 (d, 1H), 4.69-4.60 (m, 4H), 3.90 (s, 3H).

### b) 2-Allyloxy-4-chloromethyl-1-methoxy-benzene

A solution of 7.14 g (60 mmol) 1-H-benztriazole and 4.37 ml SOCl₂ (60 mmol) in 30 ml DCM is added dropwise to a stirred solution of 10 g (56.1 mmol) (3-allyloxy-4-methoxy-phenyl)-methanol in 100 ml DCM. After 10 minutes the reaction mixture is filtered, washed with sat. aq. NaHCO₃ and water, dried over Na₂SO₄ and concentrated.

Rf: (hexane/EtOAc=3/1): 0.39

¹H-NMR (400 MHz, CDCl₃): 7.0-6.86 (m, 3H), 6.19-6.07 (m, 1H), 5.46 (d, 1H), 5.35 (d, 1H), 4.68-4.63 (m, 2H), 4.59 (s, 2H), 3.91 (s, 3H).

### Building block F2: 2-Allyl-4-chloromethyl-1-methoxy-benzene

### a) 3-Allyl-4-methoxy-benzoic acid methyl ester

A mixture of 3.1 g (16.1 mmol) 3-allyl-4-hydroxy-benzoic acid methyl ester (Kasibhatla S R, Bookser B C, Probst G, Appelman J R, Erion M D J. Med. Chem. 2000, 43,1508), 4.5 g potassium carbonate (32.3 mmol) and 2.02 ml (32.3 mmol) MeI in 50 ml acetone is stirred at 25° C. for 18 h. The mixture is concentrated, taken up in water and extracted with DCM to yield 3.4 g yellowish oil that is used in the next step without purification.

Rf: (hexane/EtOAc=3/1): 0.50

LC (Nucleosil C-18HD, 4×70 mm, 3 μm, 20-100% AcCN (6 min), 100% AcCN (1.5 min)): 5.415 min

MS (ES+): 207=[M+H]⁺

¹H-NMR (400 MHz, CDCl₃): 7.96-7.84 (m, 2H), 6.90 (d, 1H), 6.09-5.96 (m, 1H), 5.13-5.07 (m, 2H), 3.90 (s, 6H), 3.42 (d, 2H).

### b) (3-Allyl-4-methoxy-phenyl)-methanol

A suspension of 1.18 g 830 mmol) LiAlH₄ in 50 ml THF is stirred at 25° C. 3-Allyl-4-methoxy-benzoic acid methyl ester (3.1 g, 15 mmol) in THF is added dropwise. The mixture is refluxed for one hour, allowed to cool down and diluted with 50 ml TBME. The excess hydride is quenched with 0.5 ml water, 0.5 ml 4N NaOH followed by 1.5 ml water. A white precipitate is filtered off and the filtrate concentrated to yield 2.7 g yellowish oil.

Rf: (hexane/EtOAc=1/1): 0.50

LC (Nucleosil C-18HD, 4×70 mm, 3 μm, 20-100% AcCN (6 min), 100% AcCN (1.5 min)): 3.963 min

MS (ES+): 161=[M-OH]⁺

¹H-NMR (400 MHz, CDCl₃): 7.26-7.18 (m, 2H), 6.87 (d, 1H), 6.09-5.97 (m, 1H), 5.12-5.06 (m, 2H), 4.63 (s, 2H), 3.85 (s, 3H), 3.41 (d, 2H).

### Building block F3: 1-Allyl-3-chloromethyl-benzene

### a) (3-Allyl-phenyl)-methanol

To a solution of 8.42 g (39.3 mmol) 3-allyl-benzoic acid ethyl ester (J. Med. Chem. 2004, 47, 5937) in 150 ml DCM stirred at 0° C. are added dropwise 100 ml diisobutylaluminium hydride (1 M solution in DCM). Stirring is continued at this temperature for 1 h before the reaction is quenched via careful addition of 20 ml methanol. The mixture is stirred at room temperature and 100 ml of a 1 M sulfuric acid is added. After 1 hour the aqueous phase is extracted twice with DCM and the combined organic layers are washed with water, dried over Na₂SO₄ and chromatographed on silica gel (EtOAc/hexanes 1:4) to give 4.53 g colorless oil.

Rf: (Hexane/EtOAc=20/1): 0.71

¹H-NMR (400 MHz, CDCl₃): 7.36-7.15 (m, 4H), 6.07-5.96 (m, 1H), 5.17-5.08 (m, 2H), 4.69 (s, 2H), 3.42 (d, 2H).

LC (Nucleosil C-18HD, 4×70 mm, 3 μm, 20-100% MeCN (6 min), 100% MeCN (1.5 min)): 3.943 min.

MS (ES+): 131=[M-OH]⁺

### b) 1-Allyl-3-chloromethyl-benzene

To a stirred solution of 6.35 g (42.8 mmol) (3-allyl-phenyl)-methanol in 65 ml DCM is added 9.4 ml SOCl₂ (129 mmol) dropwise. Two drops of pyridine are added and the mixture is stirred for three hours. The mixture is concentrated in vacuo, taken up in TBME, washed with 5% aqueous NaHCO₃, dried over Na₂SO₄ and concentrated to yield 4.92 g yellowish liquid.

Rf: (Hexane/EtOAc=20/1): 0.71

¹H-NMR (400 MHz, CDCl₃): 7.35-7.17 (m, 4H), 6.07-5.95 (m, 1H), 5.19-5.10 (m, 2H), 4.61 (s, 2H), 3.42 (d, 2H).

### Building block F4: 1-Allyloxy-3-chloromethyl-5-methyl-benzene

### a) 3-Allyloxy-5-methyl-benzoic acid methyl ester

The suspension of 8.53 g (51.3 mmol) 3-hydroxy-5-methyl-benzoic acid methyl ester (J. Org. Chem. 1959, 24, 1952), 16.2 ml (121 mmol) allyl bromide and 14.2 g (102.6 mmol) potassium carbonate in 80 ml acetone is stirred for 6 h. The reaction mixture is filtered, concentrated and taken up in EtOAc and water. The organic phase is dried over Na₂SO₄ and concentrated to give the title compound as an oil pure enough for further transformations.

Rf: (Hexane/EtOAc=3/1): 0.55

LC (Nucleosil C-18HD, 4×70 mm, 3 μm, 20-100% MeCN (6 min), 100% MeCN (1.5 min)): 5.463 min

MS (ES+): 207=[M+H]⁺

¹H-NMR (400 MHz, CDCl₃): 7.50 (s, 1H), 7.41 (s, 1H), 6.98 (s, 1H), 6.15-6.05 (m, 1H), 5.48-5.30 (m, 2H), 4.61-4.58 (m, 2H), 3.94 (s, 3H), 2.40 (s, 3H).

### b) (3-Allyloxy-5-methyl-phenyl)-methanol

To a stirred solution of 1.73 g (8.39 mmol) 3-allyloxy-5-methyl-benzoic acid methyl ester in 100 ml TBME are added 478 mg LiAlH₄ in portions. After 5 h the grey suspension is quenched with 0.5 ml water subsequently followed by 0.5 ml 4N NaOH and 1.5 ml water. After 30 min the white suspension is filtered, washed with TBME and the filtrate is concentrated to give the title compound as a colorless oil.

Rf: (Hexane/EtOAc=2/1): 0.48

LC (Nucleosil C-18HD, 4×70 mm, 3 μm, 20-100% MeCN (6 min), 100% MeCN (1.5 min)): 4.028 min

MS (ES+): 161=[M-OH]⁺

¹H-NMR (400 MHz, CDCl₃): 6.80 (s, 1H), 6.77 (s, 1H), 6.71 (s, 1H), 6.15-6.05 (m, 1H), 5.48-5.30 (m, 2H), 4.65 (s, 2H), 4.58-4.55 (m, 2H), 2.37 (s, 3H).

### c) 1-Allyloxy-3-chloromethyl-5-methyl-benzene

To a stirred solution of 3.8 g (21.3 mmol) (3-allyloxy-5-methyl-phenyl)-methanol in 65 ml DCM is added 4.66 ml SOCl₂ (64 mmol) dropwise. Two drops of pyridine are added and the mixture is refluxed for 5 h. The mixture is concentrated in vacuo, taken up in TBME, washed with 5% aqueous NaHCO₃, dried over Na₂SO₄ and chromatographed on silica gel (EtOAc/hexanes 1:20).

Rf: (Hexane/EtOAc=9/1): 0.70

¹H-NMR (400 MHz, CDCl₃): 6.83 (s, 1H), 6.80 (s, 1H), 6.74 (s, 1H), 6.16-6.06 (m, 1H), 5.48-5.32 (m, 2H), 4.60-4.55 (m, 2H), 4.56 (s, 2H), 2.38 (s, 3H).

### Building block F5: 2-Allyl-4-chloromethyl-1-fluoro-benzene

### a) 2-(3-Bromo-4-fluoro-phenyl)-[1,3]dioxolane

A mixture of 23 g (113 mmol) 3-bromo-4-fluoro-benzaldhyde and 9.5 ml (170 mmol) ethylene glycol in 150 ml c-hexane/toluene (1:1) is refluxed in the presence of 100 mg camphersulfonic acid with azeotropic removal of water. After 4 h the mixture is diluted with EtOAc and washed with 5% aq. HaHCO₃, dried over sodium sulfate, concentrated and distilled at 8 mbar, bp. 140-160° C., to give a colorless liquid (26.2 g).

¹H-NMR (400 MHz, d6-DMSO): 7.73 (s, 1H), 7.59 (s, 1H), 5.93-5.82 (m, 1H), 5.16-5.07 (m, 2H), 4.52-4.46 (m, 2H), 2.53 (s, 3H), 2.07 (s, 3H).

### b) 2-(3-Allyl-4-fluoro-phenyl)-[1,3]dioxolane

A solution of 23.9 g (96.7 mmol) 2-(3-bromo-4-fluoro-phenyl)-[1,3]dioxolane and 38 ml (122.4 mmol) allyl tributyl stannane in 250 ml DMF in the presence of 2.9 g (2.53 mmol) tetrakis(triphenyl phosphine)palladium(0) is heated at 90° C. for 18 h. After being cooled down TBME and water are added. The organic layer is stirred with 300 ml 10% aq. potassium fluoride soln. overnight. The mixture is filtered over celite and the organic layer is washed with water, dried over sodium sulfate and evaporated. Chromatography on silica gel (EtOAc/hexanes=1:9) gives the title compound as a colorless liquid.

Rf: (hexane/EtOAc=20/1): 0.17

¹H-NMR (400 MHz, CDCl₃): 7.75-771 (m, 1H), 7.48-7.40 (m, 1H), 7.17 (t, 1H), 5.80 (s, 1H), 4.18-4.04 (m, 4H).

### c) 3-Allyl-4-fluoro-benzaldehyde

To a stirred mixture of 16.6 g (81 mmol) 2-(3-allyl-4-fluoro-phenyl)-[1,3]dioxolane in 200 ml THF and 80 ml 2N aq. HCl is added acetone till the mixture becomes a homogeneous solution. The mixture is stirred till the starting material is converted. TBME is added, the organic phase is washed with 5% aq. NaHCO₃, and dried over sodium sulfate. The crude product is pure enough for further transformation.

Rf: (hexane/EtOAc=3/1): 0.45

¹H-NMR (400 MHz, CDCl₃): 9.99 (s, 1H), 7.82 7.78 (m, 2H), 7.21 (t, 1H), 6.05-5.93 (m, 1H), 5.20-5.12 (m, 2H), 3.49 (d, 2H).

### d) (3-Allyl-4-fluoro-phenyl)-methanol

To an ice-cooled solution of 14.86 g (90.5 mmol) 3-allyl-4-fluoro-benzaldehyde in 150 ml THF is added dropwise a solution of 4.6 g (120 mmol) sodium borohydride in water. After 0.5 h 65 ml 2N aq. HCl is added, after 5 min followed by 50 ml 10% aq. Na₂CO₃. The mixture is extracted with EtOAc, the organic layer washed with water, dried over sodium sulfate and evaporated.

Rf: (hexane/EtOAc=3/1): 0.17

LC (Zorbax SB-C18H, 3×30 mm, 1.8 μm, 30-100% MeCN (3.25 min), 100% MeCN (0.75 min), 100-30% MeON (0.25 min)): 1.630 min

MS (ES+): 149=[M-OH]⁺

¹H-NMR (400 MHz, CDCl₃): 7.33-7.00 (m, 3H), 6.05-5.94 (m, 1H), 5.16-5.08 (m, 2H), 4.67 (s, 2H), 3.43 (d, 2H).

### e) 2-Allyl-4-chloromethyl-1-fluoro-benzene

To a solution of 11.0 g (66.2 mmol) (3-allyl-4-fluoro-phenyl)-methanol in 100 ml DCM is added dropwise 15.7 ml SOCl₂ (199 mmol). The mixture is refluxed for 2 h and evaporated. The product is taken up in hexane, washed with sa. aq. NaHCO₃ and water, dried over sodium sulfate and concentrated.

Rf: (hexane/EtOAc=3/1): 0.82

1. A compound of the formula 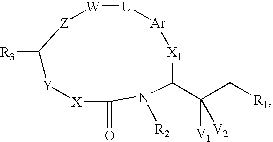
in which
R₁ is CH(Rₑ)C(═O)N(Rₐ)R_{b} or (CH₂)ₖN(R_{c})R_{d}, in which
k is 0, 1 or 2;
Rₐ and R_{b}, independently, are hydrogen or an optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl or heteroaryl(C₁₋₄)alkyl group,
R_{c} and R_{d}, independently, are hydrogen or an optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl, hetero-aryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinolin-4-yl, 1,2,3,4-tetrahydro-isoquinolin-4-yl, 1,2,3,4-tetrahydro-naphthalen-1-yl, 1,1-dioxo-1,2,3,4-tetrahydro-1lambda*6*-ben-zo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1 lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxe-pin-5-yl or 1,3,4,5-tetrahydro-benzo[c]oxepin-5-yl group, or
Rₐ and R_{b}, or R_{c} and R_{d}, together with the nitrogen to which they are attached, form an optionally substituted pyrrolidinyl, 1-piperidinyl, 4-morpholinyl or piperazinyl group; and
Rₑ is optionally substituted (C₁₋₈)alkyl, (C₁₋₄)alkoxy(C₁₋₄)alkyl, (C₃₋₇)cycloalkyl or (C₃₋₇)cycloalkyl(C₁₋₄)alkyl;
R₂ is hydrogen or (C₁₋₄)alkyl;
R₃ is hydrogen, (C₁₋₆)alkyl or an optionally substituted (C₁₋₆)alkylOC(═O)NH, (C₃₋₇)cyclo-alkylOC(═O)NH, (C₃₋₇)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₇)cycloalkylC(═O)NH, arylC(═O)NH, aryl(C₁₋₄)alkylC(═O)NH, heteroarylC(═O)NH or heteroaryl(C₁₋₄)alkylC(═O)NH group;
Ar is an aromatic or heteroaromatic ring, which ring is optionally substituted with halogen, (C₁₋₄)alkoxy, hydroxy or (C₁₋₄)alkyl, whereby U and X₁ are in ortho- or meta-position to each other;
U is a bond, —O—, CF₂, CF₂CF₂, CHF, CHFCHF, cycloprop-1,2-ylene, (C₁₋₃)alkylenoxy, (C₁₋₃)alkylenamino, (C₁₋₈)alkylene or NR_{g}, wherein
R_{g} is hydrogen, (C₁₋₈)alkyl or (C₃₋₇)cycloalkyl;
either
V₁ is hydrogen and
V₂ is hydroxy, or
V₁ and V₂ together are oxo;
W is CH═CH, cycloprop-1,2-ylene, CH₂CH(OH), CH(OH)CH₂ or CRₕRₕCRₕRₕ, wherein each Rₕ, independently, is hydrogen, fluorine or (C₁₋₄)alkyl;
X is an optionally substituted (C₁₋₄)alkanylylidene, (C₁₋₄)alkylene, (C₃₋₇)cycloalkylene, piperidin-diyl, pyrrolidin-diyl, benzothiazole-4,6-diyl, benzoxazole-4,6-diyl, 1H-benzotriazole-4,6-diyl, imidazo[1,2-a]pyridine-6,8-diyl, benzo[1,2,5]oxadiazole-4,6-diyl, benzo[1,2,5]thiadiazole-4,6-diyl, 1H-indole-5,7-diyl, 1H-indole-4,6-diyl, 1H-benzimidazole-4,6-diyl or I H-indazole-1,6-diyl group or an optionally substituted aromatic or heteroaromatic ring, whereby Y and C(═O)NR₂ are in meta-position to each other;
X₁ is CR_{f}R_{f}, wherein
each R_{f}, independently, is hydrogen, fluorine or an optionally substituted (C₁₋₈)alkyl, (C₁₋₄)alkoxy(C₁₋₄)alkyl, (C₃₋₇)cycloalkyl or (C₃₋₇)cycloalkyl(C₁₋₄)alkyl group;
Y is a bond, O, S(═O)₂, S(═O)₂NR_{g}, N(R_{g})S(═O)₂, NR_{g}, C(R_{g})OH, C(═O)NR_{g}, N(R_{g})C(═O), C(═O)N(R_{g})O or ON(R_{g})C(═O), wherein
R_{g} is hydrogen, (C₁₋₈)alkyl or (C₃₋₇)cycloalkyl; and
Z is O, CH₂, CF₂, CHF, cycloprop-1,2-ylene or a bond,
the number of ring atoms included in the macrocyclic ring being 14, 15, 16 or 17, in free base form or in acid addition salt form.

2. A process for the preparation of a compound as defined in claim 1 of the formula I, in free base form or in acid addition salt form, comprising the steps of
a) cyclisation by metathesis of a compound of the formula 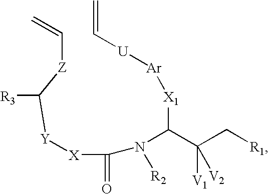
in which all of the variables are as defined for the formula I, in the presence of a catalyst, for instance a ruthenium, tungsten or molybdenum complex, or
b) for the preparation of a compound of the formula I, in which R₁ is CH(Rₑ)C(═O)N(Rₐ)R_{b}, V₁ is hydrogen and V₂ is hydroxy, reaction of a compound of the formula 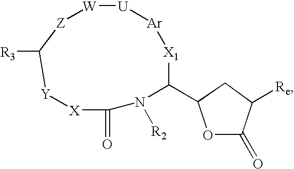
in which all of the variables are as defined for the formula I, with a compound of the formula HN(Rₐ)R_{b} (IV), in which Rₐ, and R_{b} are as defined for the formula I, or
c) for the preparation of a compound of the formula I, in which W is CH₂CH₂, hydrogenation of a compound of the formula 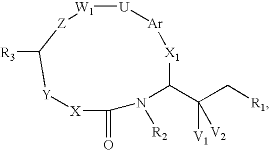
in which W₁ is CH═CH and all of the other variables are as defined for the formula I, or
d) for the preparation of a compound of the formula I, in which R₁ is N(H)R_{d} (in which R_{d}, if it is hydrogen, may be protected by a protecting group to be removed subsequently), V₁ is hydrogen and V₂ is hydroxy, cleavage of the C═O function in the moiety O—C(═O)—NR_{d} in a compound of the formula 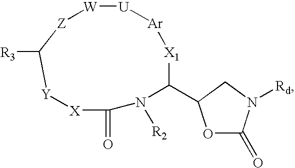
in which all of the variables are as defined for the formula I (and R_{d}, if it is hydrogen, may be protected by a protecting group to be removed subsequently), using, for instance, barium hydroxide or cesium carbonate, or
e) for the preparation of a compound of the formula I, in which R₁ is N(R_{c})R_{d}, V₁ is hydrogen and V₂ is hydroxy, reaction of a compound of the formula 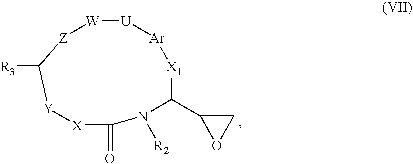
in which all of the variables are as defined for the formula I, with a compound of the formula HN(R_{c})R_{d} (VIII), in which R_{c} and R_{d} are as defined for the formula I,
in each case optionally followed by reduction, oxidation or functionalisation of the resulting compound and/or by cleavage of protecting groups optionally present, and of recovering the so obtainable compound of the formula I in free base form or in acid addition salt form.

3. A compound according to claim 1 of the formula I, in free base form or in pharmaceutically acceptable acid addition salt form, for use as a medicament.

4. A compound according to claim 1 of the formula I, in free base form or in pharmaceutically acceptable acid addition salt form, for use in the treatment of neurological or vascular disorders related to beta-amyloid generation and/or aggregation.

5. A pharmaceutical composition comprising a compound as claimed in claim 1 of the formula I, in free base form or in pharmaceutically acceptable acid addition salt form, as active ingredient and a pharmaceutical carrier or diluent.

6. The use of a compound as claimed in claim 1 of the formula I, in free base form or in pharmaceutically acceptable acid addition salt form, as a medicament for the treatment of neurological or vascular disorders related to beta-amyloid generation and/or aggregation.

7. The use of a compound as claimed in claim 1 of the formula I, in free base form or in pharmaceutically acceptable acid addition salt form, for the manufacture of a medicament for the treatment of neurological or vascular disorders related to beta-amyloid generation and/or aggregation.

8. A method for the treatment of neurological or vascular disorders related to beta-amyloid generation and/or aggregation in a subject in need of such treatment, which comprises administering to such subject a therapeutically effective amount of a compound as claimed in claim 1 of the formula I, in free base form or in pharmaceutically acceptable acid addition salt form.

9. A combination comprising a therapeutically effective amount of a compound as claimed in claim 1 of the formula I, in free base form or in pharmaceutically acceptable acid addition salt form, and a second drug substance, for simultaneous or sequential administration.
